SEQUENCE LISTING (21 sequences)

SEQ ID NO: 1 - DNA, 2917 bp, Homo sapiens
gcagacacctgggctgagacatacaggacagagcatggatcgcctacagactgcactcctggttgtcctcgtcctccttgctgtggcgcttcaagcaactgaggcaggcccctacggcgccaacatggaagacagcgtctgctgccgtgattacgtccgttaccgtctgcccctgcgcgtggtgaaacacttctactggacctcagactcctgcccgaggcctggcgtggtgttgctaaccttcagggataaggagatctgtgccgatcccagagtgccctgggtgaagatgattctcaataagctgagccaatgaagagcctactctgatgaccgtggccttggctcctccaggaaggctcaggagccctacctccctgccattatagctgctccccgccagaagcctgtgccaactctctgcattccctgatctccatccctgtggctgtcacccttggtcacctccgtgctgtcactgccatctcccccctgacccctctaacccatcctctgcctccctccctgcagtcagagggtcctgttcccatcagcgattcccctgcttaaacccttccatgactccccactgccctaagctgaggtcagtctcccaagcctggcatgtggccctctggatctgggttccatctctgtctccagcctgcccacttcccttcatgaatgttgggttctagctccctgttctccaaacccatactacacatcccacttctgggtctttgcctgggatgttgctgacacccagaaagtcccaccacctgcacatgtgtagccccaccagccctccaaggcattgctcgcccaagcagctggtaattccatttcatgtattagatgtcccctggccctctgtcccctcttaataaccctagtcacagtctccgcagattcttgggatttgggggttttctcccccacctctccactagttggaccaaggtttctagctaagttactctagtctccaagcctctagcatagagcactgcagacaggccctggctcagaatcagagcccagaaagtggctgcagacaaaatcaataaaactaatgtccctcccctctccctgccaaaaggcagttacatatcaatacagagactcaaggtcactagaaatgggccagctgggtcaatgtgaagccccaaatttgcccagattcacctttcttcccccactccctttttttttttttttttgagatggagtttcgctcttgtcacccacgctggagtgcaatggtgtggtcttggcttattgaagcctctgcctcctgggttcaagtgattctcttgcctcagcctcctgagtagctgggattacaggttcctgctaccacgcccagctaatttttgtatttttagtagagacgaggcttcaccatgttggccaggctggtctcgaactcctgtcctcaggtaatccgcccacctcagcctcccaaagtgctgggattacaggcgtgagccacagtgcctggcctcttccctctccccaccccccccccaacttttttttttttttatggcagggtctcactctgtcgcccaggctggagtgcagtggcgtgatctcggctcactacaacctcgacctcctgggttcaagcgattctcccaccccagcctcccaagtagctgggattacaggtgtgtgccactacggctggctaatttttgtatttttagtagagacaggtttcaccatattggccaggctggtcttgaactcctgacctcaagtgatccaccttccttgtgctcccaaagtgctgagattacaggcgtgagctatcacacccagcctccccctttttttcctaataggagactcctgtacctttcttcgttttacctatgtgtcgtgtctgcttacatttccttctcccctcaggctttttttgggtggtcctccaacctccaatacccaggcctggcctcttcagagtaccccccattccactttccctgcctccttccttaaatagctgacaatcaaattcatgctatggtgtgaaagactacctttgacttggtattataagctggagttatatatgtatttgaaaacagagtaaatacttaagaggccaaatagatgaatggaagaattttaggaactgtgagagggggacaaggtggagctttcctggccctgggaggaagctggctgtggtagcgtagcgctctctctctctgtctgtggcaggaggcaaagagtagggtgtaattgagtgaaggaatcctgggtagagaccattctcaggtggttgggccaggctaaagactgggatttgggtctatctatgcctttctggctgatttttgtagagacggggttttgccatgttacccaggctggtctcaaactcctgggctcaagcgatcctcctggctcagcctcccaaagtgctgggattacaggcgtgagtcactgcgcctggcttcctcttcctcttgagaaatattcttttcatacagcaagtatgggacagcagtgtcccaggtaaaggacataaatgttacaagtgtctggtcctttctgagggaggctggtgccgctctgcagggtatttgaacctgtggaattggaggaggccatttcactccctgaacccagcctgacaaatcacagtgagaatgttcaccttataggcttgctgtggggctcaggttgaaagtgtggggagtgacactgcctaggcatccagctcagtgtcatccagggcctgtgtccctcccgaacccagggtcaacctgcctaccacaggcactagaaggacgaatctgcctactgcccatgaacggggccctcaagcgtcctgggatctccttctccctcctgtcctgtccttgcccctcaggactgctggaaaataaatcctttaaaatag

SEQ ID NO: 2 - DNA, 2527 bp, Homo sapiens
agaacccacaaagcctgcccctcatcccaggcagagagcaacccagctctttccccagacactgagagctggtggtgcctgctgtcccagggagagttgcatcgccctccacagagcaggcttgcatctgactgacccaccatgacacccacagacttcacaagccctattcctaacatggctgatgactatggctctgaatccacatcttccatggaagactacgttaacttcaacttcactgacttctactgtgagaaaaacaatgtcaggcagtttgcgagccatttcctcccacccttgtactggctcgtgttcatcgtgggtgccttgggcaacagtcttgttatccttgtctactggtactgcacaagagtgaagaccatgaccgacatgttccttttgaatttggcaattgctgacctcctctttcttgtcactcttcccttctgggccattgctgctgctgaccagtggaagttccagaccttcatgtgcaaggtggtcaacagcatgtacaagatgaacttctacagctgtgtgttgctgatcatgtgcatcagcgtggacaggtacattgccattgcccaggccatgagagcacatacttggagggagaaaaggcttttgtacagcaaaatggtttgctttaccatctgggtattggcagctgctctctgcatcccagaaatcttatacagccaaatcaaggaggaatccggcattgctatctgcaccatggtttaccctagcgatgagagcaccaaactgaagtcagctgtcttgaccctgaaggtcattctggggttcttccttcccttcgtggtcatggcttgctgctataccatcatcattcacaccctgatacaagccaagaagtcttccaagcacaaagccctaaaagtgaccatcactgtcctgaccgtctttgtcttgtctcagtttccctacaactgcattttgttggtgcagaccattgacgcctatgccatgttcatctccaactgtgccgtttccaccaacattgacatctgcttccaggtcacccagaccatcgccttcttccacagttgcctgaaccctgttctctatgtttttgtgggtgagagattccgccgggatctcgtgaaaaccctgaagaacttgggttgcatcagccaggcccagtgggtttcatttacaaggagagagggaagcttgaagctgtcgtctatgttgctggagacaacctcaggagcactctccctctgaggggtcttctctgaggtgcatggttcttttggaagaaatgagaaatacagaaacagtttccccactgatgggaccagagagagtgaaagagaaaagaaaactcagaaagggatgaatctgaactatatgattacttgtagtcagaatttgccaaagcaaatatttcaaaatcaactgactagtgcaggaggctgttgattggctcttgactgtgatgcccgcaattctcaaaggaggactaaggaccggcactgtggagcaccctggctttgccactcgccggagcatcaatgccgctgcctctggaggagcccttggattttctccatgcactgtgaacttctgtggcttcagttctcatgctgcctcttccaaaaggggacacagaagcactggctgctgctacagaccgcaaaagcagaaagtttcgtgaaaatgtccatctttgggaaattttctaccctgctcttgagcctgataacccatgccaggtcttatagattcctgatctagaacctttccaggcaatctcagacctaatttccttctgttctccttgttctgttctgggccagtgaaggtccttgttctgattttgaaacgatctgcaggtcttgccagtgaacccctggacaactgaccacacccacaaggcatccaaagtctgttggcttccaatccatttctgtgtcctgctggaggttttaacctagacaaggattccgcttattccttggtatggtgacagtgtctctccatggcctgagcagggagattataacagctgggttcgcaggagccagccttggccctgttgtaggcttgttctgttgagtggcacttgctttgggtccaccgtctgtctgctccctagaaaatgggctggttcttttggccctcttctttctgaggcccactttattctgaggaatacagtgagcagatatgggcagcagccaggtagggcaaaggggtgaagcgcaggccttgctggaaggctatttacttccatgcttctccttttcttactctatagtggcaacattttaaaagcttttaacttagagattaggctgaaaaaaataagtaatggaattcacctttgcatcttttgtgtctttcttatcatgatttggcaaaatgcatcacctttgaaaatatttcacatattggaaaagtgctttttaatgtgtatatgaagcattaattacttgtcactttctttaccctgtctcaatattttaagtgtgtgcaattaaagatcaaatagatacat

SEQ ID NO: 3 - DNA, 828 bp, Homo sapiens
atgccgtacgccaaccagcctaccgtgcggatcacggagctcactgacgagaatgtcaagttcatcatcgagaacaccgacctggcggtggccaattcgattcggagggtcttcatcgctgaggttcccataatagccattgactgggttcagattgatgccaattcctcagtccttcatgatgaattcattgctcacaggcttggattaattcccctcattagtgatgacattgtggacaagctgcagtactctcgggactgcacatgtgaggagttctgccccgagtgctcggtggagttcaccctcgatgtgcggtgcaatgaagaccagacgcgacatgtcacgtctcgagacctcatctccaacagcccccgggtcattccggtgacatcccggaaccgagataatgaccccaatgactacgtggagcaggatgacatcctcatcgtcaagttgagaaagggccaggagctgagacttcgagcctatgccaaaaagggctttggcaaggagcatgccaagtggaaccctactgcaggggtggcttttgaatacgatccagacaatgccctgaggcacacagtgtaccccaagcccgaggaatggccaaagagtgagtactcggagctggatgaggatgagtcgcaggctccctatgaccccaacggcaagccagaaaggttttactacaatgtggagtcctgtggctctctgcgtcctgaaaccattgtcctgtcagccctctcaggattgaagaagaaactgagtgatttacaaactcaattaagccacgagatccagagtgatgtgctaaccataaattaa

SEQ ID NO: 4 - DNA, 1974 bp, Homo sapiens
agcggcgtaaatattgagttaacctctggaagcaggctttgactttgatattgacctgaccacttaactctctggagactttgacaagtgacatcaatgtctgtctctgtgtttcctgatttatatccacagatagaaagccatctccagatcctagggccattttcaagattcagtgagataaggtgaagcatttgaatgttctccataaatgtcagctgcttttattattactagcattattttttgttttcattattattattaacatagacccgtctaagggcagaaagaaatctgaccaagaatggagtgaaagtcggctgtgaaggttaggactccctgaaaacaaaaaagaaaagtggtaacatcactctgtatccaagggcatggataaggatgattcagaatttttttataacattgatgttaatatacatagagaaatacacacacacacacacgtatatatgtccatggacatcaaaatgaggtataggttaggaagttaccctggaagttagaattgagataattactttgattagcaaaggtttatgttagaaatacgagtggttgaaggcagcgcctttcaccttgatgcctgttgtgtttagcactggatagaaaattgctatttttactgatgactactttaatgcttggttcattcatatgaaataatctttcttttctcctttaataataatgatctctcctgatttcaagttgaaaatgtattaatttaagataacttatgaagcaatgacatatggaagaggagatgggaaattgtttctcaatgggaaagtgtttataaatgggataggtatactaaaggtatttccatttagggaaagaggaaaggaatggtattaatatatttctctaatatttttccccctttgaaagtttgccatgttaggaatcctggggatggagtgatgaattgtggcattgctctgcagttcacgctgctgagagctgccccaacacacacacacacacacacacacacacacacacacaaaggttgcacacacacaaacaggtttgattatgctgatgagacttcttttattattgaaaatatcttaaagttttatctcaccaaggaggccttttctctttccctctctttctagaaggccaatgatgaggccaatcagagtgatacaagtgtctccttgtcagaacccaagagcaaaagcagccttcacttactgtcccatgaaacaaaaattggatcttttctaagcaacagaactttagatggcaaagacaaagctggcctttgtccagatgaagatgatatggaaggagattctttctttgatgatcccattcctaagccagagaaaacttacggtttgaggaaggaacctaggaagcaagcaggaagtctggcctcgctctcggatgcaccccccttaaaaagtggactcagctccctggcgggagccccttctttaaaagactctgagagtaaaaggggaaatacagttttgaaagatctgaaattgatcagtgataaaattggatcacttggattaggaactggagaagatgatgactatgttgatgattttaatagtaccagccatcgctcagagaaaagtgagataagtattggtgaagagatagaagaagacctttctgtggaaatagatgacatcaataccagtgataagacaatcactcagctggaatgtctgctctctattggtgccttgcatttcaaaaacactgcagatattttttaaaagtaattttcattttactaaacaaaatacttcctatttgagcccatgtgtggaagatttaatattcttaatttaactgtacatttctttatggaaattgattatctacactcagtttcattacagggaaggaacccatgaaaacatcagtgttaagagcatgatgaaaggtgtcaataaagccgtaggatcgcacaaaaaaaaaaaaaaa

SEQ ID NO: 5 - DNA, 3979 bp, Homo sapiens
ggcggccgcggcagggcgggcgccgcgcggaggcagggcgggcgtattcaatggaagtgtgttaccagctgccggtactgcccctggacaggccggtcccccagcacgtcctcagccgccgaggagccatcagcttcagctccagctccgctctcttcggctgccccaatccccggcagctctctcagaggcgtggagctatttcctatgacagttctgatcagactgcattatacattcgtatgctaggagatgtacgtgtaaggagccgagcaggatttgaatcagaaagaagaggttctcacccatatattgattttcgtattttccactctcaatctgaaattgaagtgtctgtctctgcaaggaatatcagaaggctactaagtttccagcgatatcttagatcttcacgcttttttcgtggtactgcggtttcaaattccctaaacattttagatgatgattataatggacaagccaagtgtatgctggaaaaagttggaaattggaattttgatatctttctatttgatagactaacaaatggaaatagtctagtaagcttaacctttcatttatttagtcttcatggattaattgagtacttccatttagatatgatgaaacttcgtagatttttagttatgattcaagaagattaccacagtcaaaatccttaccataacgcagtccacgctgcggatgttactcaggccatgcactgttacttaaaggaacctaagcttgccaattctgtaactccttgggatatcttgctgagcttaattgcagctgccactcatgatctggatcatccaggtgttaatcaacctttccttattaaaactaaccattacttggcaactttatacaagaatacctcagtactggaaaatcaccactggagatctgcagtgggcttattgagagaatcaggcttattctcacatctgccattagaaagcaggcaacaaatggagacacagataggtgctctgatactagccacagacatcagtcgccagaatgagtatctgtctttgtttaggtcccatttggatagaggtgatttatgcctagaagacaccagacacagacatttggttttacagatggctttgaaatgtgctgatatttgtaacccatgtcggacgtgggaattaagcaagcagtggagtgaaaaagtaacggaggaattcttccatcaaggagatatagaaaaaaaatatcatttgggtgtgagtccactttgcgatcgtcacactgaatctattgccaacatccagattggttttatgacttacctagtggagcctttatttacagaatgggccaggttttccaatacaaggctatcccagacaatgcttggacacgtggggctgaataaagccagctggaagggactgcagagagaacagtcgagcagtgaggacactgatgctgcatttgagttgaactcacagttattacctcaggaaaatcggttatcataacccccagaaccagtgggacaaactgcctcctggaggtttttagaaatgtgaaatggggtcttgaggtgagagaacttaactcttgactgccaaggtttccaagtgagtgatgccagccagcattatttatttccaagatttcctctgttggatcatttgaacccacttgttaattgcaagacccgaacatacagcaatatgaatttggctttcatgtgaaaccttgaatataaagcccagcaggagagaatccgaaaggagtaacaaaggaagttttgatatgtgccacgactttttcaaagcatctaatcttcaaaacgtcaaacttgaattgttcagcaacaatctcttggaatttaaccagtctgatgcaacaatgtgtatcttgtaccttccactaagttctctctgagaaaatggaaatgtgaagtgcccagcctctgctgcctctggcaagacaatgtttacaaatcaactctgaaaatattggttctaaattgccttggagcatgattgtgaaggaaccactcaaacaaatttaaagatcaaactttagactgcagctctttccccctggtttgcctttttcttctttggatgccaccaaagcctcccatttgctatagttttatttcatgcactggaaactgagcatttatcgtagagtaccgccaagctttcactccagtgccgtttggcaatgcaattttttttagcaattagtttttaatttggggtgggaggggaagaacaccaatgtcctagctgtattatgattctgcactcaagacattgcatgttgttttcactactgtacacttgacctgcacatgcgagaaaaaggtggaatgtttaaaacaccataatcagctcaggtatttgccaatctgaaataaaagtgggatgggagagcgtgtccttcagatcaagggtactaaagtccctttcgctgcagtgagtgagaggtatgttgtgtgtgaatgtacggatgtgtgtttggtgatgtttgtgcatgtgtgacgtgcatgttatgtttctccatgtgggcaaagatttgaaagtaagcttttatttattattttagaatgtgacataatgagcagccacactcgggggaggggaaggttggtaggtaagctgtaacagattgctccagttgccttaaactatgcacatagctaagtgaccaaacttcttgttttgatttgaaaaaagtgcattgttttcttgtccctccctttgatgaaacgttaccctttgacgggccttttgatgtgaacagatgttttctaggacaaactataaggactaattttaaacttcaaacattccacttttgtaatttgttttaaattgttttatgtatagtaagcacaactgtaatctagttttaagagaaaccggtgctttcttttagttcatttgtatttcccttgttactgtaaaagactgtttattaattgtttacagtttgttgcaacagccattttcttgggagaaagcttgagtgtaaagccatttgtaaaaggctttgccatactcattttaatatgtgcctgttgctgttaacttttgatgaataaaaacctatcttttcatgaaacttctctctatacaaattgaaatacataatgctttctggttcttcttcaaaccaaaacttgtcaaattcatagacaagataacagtaaaactgatgaaagtgttccattgttggtataccaggaacaaggttatagagatgaaacttcaaagcttcactcttcagtaagctataagccatctctgtaagattgattccaactattgcataagaataccctaattttggatgatttgaacgggaaagaatctgatgagcttcactagtgtaattttcactgaaatacacaagattgattaacccaagtatgcccatgcctctgaagtctgtcttgggatcatcaccctgaaaaccaatttcagcccactgcttggagattctagcgtttaacttcttcgtgggcattagaagattccaaagcttcatgagtagctcttcatgctgtaggttatcagaatcatatggccttttcctcacactttctacatccaaatacagctgtttataaccagttatctgcagtaagcacatcttcatgcatattttaaaactggcatccttctcagggttaatattcttttccttcataatatcatctacatatttgtccacttcactctgaacaacatgtgtcgccttctgtaaaaccttattcttggagtatgtcaaggaattttctatcctgtgtgtcctttgtgcacctacataggtatcaaatattcgctgcaattcacacttcccagtcatctgtcgtaatagccatttcatccaaaatcgaaaaaagtgcccatagaagaactcccacaaagaaataaacatttttttttcctcacaggagcggaagaactagggggagcaggagctgcaatgcggccgc

SEQ ID NO: 6 - DNA, 636 bp, Homo sapiens
atggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagattggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatccgcactgccacagagaagccgctgggggagctatggaag

SEQ ID NO: 7 - DNA, 1582 bp, Homo sapiens
ctctgggcttccgtcctccgcccgcgcccgacggagcctgttcgcgtcgactgcccagagtccgcgaatcctccgctccgagcccgtccggactcccccgatcccagctttctctcctttgaaaacactaagaataatgtcactgcatcagtttttactagagccaatcacctgtcatgcctggaacagggatcgtactcagattgccctcagtcccaataatcacgaagtgcacatctataagaagaacgggagccagtgggtgaaagctcatgaactcaaggagcacaacggacacatcacaggtattgactgggctcccaagagcgaccgcattgtcacttgtggggcagaccgcaatgcctatgtctggagtcagaaagatggtgtttggaagccaaccctggtgatcctgagaattaatcgcgcagctacttttgtgaagtggtcccccctagagaacaaatttgctgtgggaagtggagcacgactcatttctgtttgttactttgagtctgaaaatgactggtgggtgagcaagcacattaaaaagccgattcgctccacagtcctcagcttggattggcatcccaacaacgttttgctggcagcaggatcatgtgacttcaaatgcagagtgttttctgcctacattaaagaagtggatgaaaagccagccagcacgccctggggcagcaagatgccttttgggcagctgatgtcagagtttggtggcagtggcactggtggctgggtccacggggtaagcttctctgccagtgggagccgcctggcctgggtcagccacgacagcaccgtgtctgttgctgatgcctcaaaaagtgtgcaggtctcgactctgaagacagagttcctgccgctcctaagtgtgtcatttgtctcagagaacagcgtcgtggctgctggccatgactgctgcccaatgctctttaactacgatgaccgcggctgcctgaccttcgtctccaagttagatattccaaaacagagcatccaacgcaacatgtctgccatggaacgcttccgcaacatggacaagagagccacaactgaggaccgcaacacggccttggagacgctgcaccagaatagcatcactcaagtctctatttatgaggtggacaagcaagattgtcgcaaattttgcactactggcatcgatggagccatgacaatttgggatttcaagaccctcgagtcttccatccagggcctccggataatgtgaagctgagtgagcctccgccatccagcatgacaaactgtggccgaccgcagctgtgccgtggcacgatggcgaggaagccagccccaaggaaacactgaaaacacatatcacgccaatgccgtgtggttttgtttgaatataaaattggtgaaagtgttggtttttttaaggcagtaatttttttgtttgtttttttgcgatttcattccattcttgaccaaagcttctctttaagtagtttattatggaaaattgtcacactaacttaaaagacagggtgagggagatatgtaaattgtccactagaaaattaaataaaagaactgaatgtgg

SEQ ID NO: 8 - DNA, 462 bp, Homo sapiens
ccttttcctccctgtcgccaccgaggtcgcacgcgtgagacttctccgccgcctccgccgcagacgccgccgcgatgcgctacgtcgcctcctacctgctggctgccctagggggcaactcctcccccagcgccaaggacatcaagaagatcttggacagcgtgggtatcgaggcggacgacgaccggctcaacaaggttatcagtgagctgaatggaaaaaacattgaagacgtcattgcccagggtattggcaagcttgccagtgtacctgctggtggggctgtagccgtctctgctgccccaggctctgcagcccctgctgctggttctgcccctgctgcagcagaggagaagaaagatgagaagaaggaggagtctgaagagtcagatgatgacatgggatttggcctttttgattaaattcctgctcccctgcaaataaagcctttttacacatctc

SEQ ID NO: 9 - DNA, 1097 bp, Homo sapiens
aagtgctgcgagccctgggccacgctggccgtgctggcagtgggccgcctcgatccctctgcagtctttcccttgaggctccaagaccagcaggtgaggcctcgcggcgctgaaaccgtgaggcccggaccacaggctccagatggaccctgggaaggacaaagagggggtgccccagccctcagggccgccagcaaggaagaaatttgtgatacccctcgacgaggatgaggtccctcctggagtggccaagcccttattccgatctacacagagccttcccactgtggacacctcggcccaggcggcccctcagacctacgccgaatatgccatctcacagcctctggaaggggctggggccacgtgccccacagggtcagagcccctggcaggagagacgcccaaccaggccctgaaacccggggcaaaatccaacagcatcattgtgagccctcggcagaggggcaatcccgtactgaagttcgtgcgcaacgtgccctgggaatttggcgacgtaattcccgactatgtgctgggccagagcacctgtgccctgttcctcagcctccgctaccacaacctgcacccagactacatccatgggcggctgcagagcctggggaagaacttcgccttgcgggtcctgcttgtccaggtggatgtgaaagatccccagcaggccctcaaggagctggctaagatgtgtatcctggccgactgcacattgatcctcgcctggagccccgaggaagctgggcggtacctggagacctacaaggcctatgagcagaaaccagcggacctcctgatggagaagctagagcaggacttcgtctcccgggtgactgaatgtctgaccaccgtgaagtcagtcaacaaaacggacagtcagaccctcctgaccacatttggatctctggaacagctcatcgccgcatcaagagaagatctggccttatgcccaggcctgggccctcagaaagcccggaggctgtttgatgtcctgcacgagcccttcttgaaagtaccctgatgaccccagctgccaaggaaacccccagtgtaataataaatcgtcctcccaggccaggctc

SEQ ID NO: 10 - DNA, 995 bp, Homo sapiens
gtcgacggcagcggcggcggcgggtgggaaatggcggagtatctggcctccatcttcggcaccgagaaagacaaagtcaactgttcattttatttcaaaattggagcatgtcgtcatggagacaggtgctctcggttgcacaataaaccgacgtttagccagaccatcttgattcaaaacatctatcgtaatccccaaaacagtgcacagacggctgacggctcacactaccattgccctcttgaacatttaccgtaaccctcaaaactcttcccagtctgctgacggtttgcgctgtgccgtgagcgatgtggagatgcaggaacactatgatgagttttttgaggaggtttttacagaaatggaggagaagtatagggaagtagaggagatgaacgtctgtgacaacctgggagaccacctggtggggaacgtgtacgtcaagtttcgccgtgaggaagatgcggaaaaggctgtgattgacttgaataaccgttggtttaatggacagccgatccacgccgagctgtcacccgtgacggacttcagagaagcctgctgccgtcagtatgagatgggagaatgcacacgaggcggcttctgcaacttcatgcatttgaagcccatttccagagagctgcggcgggagctgtatggccgccgtcgcaagaagcatagatcaagatcccgatcccgggagcgtcgttctcggtctagagaccgtggtcgtggcggtggcggtggcggtggtggaggtggcggcggacgggagcgtgacaggaggcggtcgagagatcgtgaaagatctgggcgattctgagccatgccatttttaccttatgtctgctagaaagtgttgtagttgattgaccaaaccagttcataaggggaattttttttaaaaaacaacaaaaaaaaaaacatacaaagatgggtttctgaataaaatttgtagtgataacaccaaaaaaaaaaaaaaaaaaaaaaaaaaaaaa

SEQ ID NO: 11 - DNA, 4168 bp, Homo sapiens
tgttggagttggtggtggcttaagttttgaagggaggtagcatccgttggatatccacaccatccttctcgctgcaggctttcttggactccgtactgttggtgtaaccaaggcctggaggtctgggtggctcaggtttcctgcagccatgtttctgtacaacttaaccttgcagagagccactggcatcagctttgccattcatggaaacttttctggaaccaaacaacaagaaattgttgtttcccgtgggaagatcttggagctgcttcgcccagaccccaacactggcaaagtacataccctactcactgtggaagtattcggtgttatccggtcactcatggcctttaggctgacaggtggcaccaaagactacattgtagttggcagtgactctggtcgaattgttattttggaataccagccatctaagaatatgtttgagaagattcaccaagaaacctttggcaagagtggatgccgtcgcatcgttcctggccagttcttagctgtggatcccaaagggcgagccgttatgattagtgccattgagaaacagaaattggtgtatattttgaacagagatgctgcagcccgacttaccatttcatctcccctggaagcccacaaagcaaacactttagtgtatcatgtagttggagtagatgtcggatttgaaaatccaatgtttgcttgtctggaaatggattatgaggaagcagacaatgatccaacaggggaagcagcagctaatacccagcagacacttactttctatgagctagaccttggtttaaatcatgtggtccgaaaatacagtgaacctttggaggaacacggcaacttccttattacagttccaggagggtcagatggtccaagtggagtactgatctgctctgaaaactatattacttacaagaactttggtgaccagccagatatccgctgtccaattcccaggaggcggaatgacctggatgaccctgaaagaggaatgatttttgtctgctctgcaacccataaaaccaaatcgatgttcttctctttggctcaaactgagcagggagatatctttaagatcactttggagacagatgaagatatggttactgagatccggctcaaatattttgatactgtacccgttgctgctgccatgtgtgtgcttaaaacagggttcctttttgtagcatcagaatttggaaaccattacttatatcaaattgcacatcttggagatgatgatgaagaacctgagttttcatcagccatgcctctggaagaaggagacacattcttttttcagccaagaccacttaaaaaccttgtgctggttgatgagttggacagcctctctcccattctgttttgccagatagctgatctggccaatgaagatactccacagttgtatgtggcctgtggtaggggaccccgatcatctctgagagtcctaagacatggacttgaggtgtcagaaatggctgtttctgagctacctggtaaccccaacgctgtctggacagtgcgtcgacacattgaagatgagtttgatgcctacattattgtgtctttcgtgaatgccaccctagtgttgtccattggagaaactgtagaagaagtgactgactctgggttcctggggaccaccccgaccttgtcctgctccttattaggagatgatgccttggtgcaggtctatccagatggcattcggcacatacgagcagacaagagagtcaatgagtggaagacccctggaaagaaaacaattgtgaagtgtgcagtgaaccagcgacaagtggtgattgccctgacaggaggagagctggtctatttcgagatggatccttcaggacagctgaatgagtacacagaacggaaggagatgtcagcagatgtggtgtgcatgagtctggccaatgtaccccctggagagcagcggtctcgcttcctggctgtggggcttgtggacaacactgtcagaatcatctccctggatccctcagactgtttgcaacctctaagcatgcaggctctcccagcccagcctgagtccttgtgtatcgtggaaatgggtgggactgagaagcaggatgagctgggtgagaggggctcgattggcttcctatacctgaatattgggctacagaacggtgtgctgctgaggactgtcttggaccctgtcactggggatttgtctgatactcgcactcggtacctggggtcccgtcctgtgaagctcttccgagtccgaatgcaaggccaggaggcagtattggccatgtcaagccgctcatggttgagctattcttaccaatctcgcttccatctcaccccactgtcttacgagacactggaatttgcatcgggttttgcctcggaacagtgtcccgagggcattgtggccatctccaccaacaccctacggattttggcattagagaagctcggtgctgtcttcaatcaagtagccttcccactgcagtacacacccaggaaatttgtcatccaccctgagagtaacaaccttattatcattgaaacggaccacaatgcctacactgaggccacgaaagctcagagaaagcagcagatggcagaggaaatggtggaagcagcaggggaggatgagcgggagctggccgcagagatggcagcagcattcctcaatgaaaacctccctgaatccatctttggagctcccaaggctggcaatgggcagtgggcctctgtgatccgagtgatgaatcccattcaagggaacacactggaccttgtccagctggaacagaatgaggcagcttttagtgtggctgtgtgcaggttttccaacactggtgaagactggtatgtgctggtgggtgtggccaaggacctgatactaaacccccgatctgtggcagggggcttcgtctatacttacaagcttgtgaacaatggggaaaaactggagtttttgcacaagactcctgtggaagaggtccctgctgctattgccccattccaggggagggtgttgattggtgtggggaagctgttgcgtgtctatgacctgggaaagaagaagttactccgaaaatgtgagaataagcatattgccaattatatctctgggatccagactatcggacatagggtaattgtatctgatgtccaagaaagtttcatctgggttcgctacaagcgtaatgaaaaccagcttatcatctttgctgatgatacctacccccgatgggtcactacagccagcctcctggactatgacactgtggctggggcagacaagtttggcaacatatgtgtggtgaggctcccacctaacaccaatgatgaagtagatgaggatcctacaggaaacaaagccctgtgggaccgtggcttgctcaatggggcctcccagaaggcagaggtgatcatgaactaccatgtcggggagacggtgctgtccttgcagaagaccacgctgatccctggaggctcagaatcacttgtctataccaccttgtctggaggaattggcatccttgtgccattcacgtcccatgaggaccatgacttcttccagcatgtggaaatgcacctgcggtctgaacatccccctctctgtgggcgggaccacctcagctttcgctcctactacttccctgtgaagaatgtgattgatggagacctctgtgagcagttcaattccatggaacccaacaaacaaaagaacgtctctgaagaactggaccgaaccccacccgaagtgtccaagaaactcgaggatatccggacccgctacgccttctgagccctcctttcccggtggggcttgccagagactgtgtgttttgtttcccccaccaccatcactgccacctggcttctgccatgtggcaggagggtgactggataattaagactgcattatgaaagtcaacagctctttcccctcagctcttctcctggaatgactggcttcccctcaaattggcactgagatttgctacacttctccccacctggtacatgatacatgaccccaggttccagtgtagaacctgagtcccccattccccaaagccatccctgcattgatatgtcttgactctcctgtctacttttgcacacacccttaatttttaattggttttcttgtaaaaaaaaaaaaaaaaaa

SEQ ID NO: 12 - DNA, 3047 bp, Homo sapiens
gacggcgacactttgctacggagtgcatcggacgtcgaagcctagagtctctgcgtctttccctcttccgctgcctcattcctttccttcctagccttggtcgtcgccgccaccatgaacaagaagaagaaaccgttcctagggatgcccgcgcccctcggctacgtgccggggctgggccggggcgccactggcttcaccacgcggtcagacattgggcccgcccgtgatgcaaatgaccctgtggatgatcgccatgcacccccaggcaagagaaccgttggggaccagatgaagaaaaatcaggctgctgacgatgacgacgaggatctaaatgacaccaattacgatgagtttaatggctatgctgggagcctcttctcaagtggaccctacgagaaagatgatgaggaagcagatgctatctatgcagccctggataaaaggatggatgaaagaagaaaagaaagacgggagcaaagggagaaagaagaaatagagaaatatcgtatggaacgccccaaaatccaacagcagttctcagacctcaagaggaagttggcagaagtcacagaagaagagtggctgagcatccccgaggttggcgatgccagaaataaacgtcagcggaacccacgctatgagaagctgacccctgttcctgacagtttctttgccaaacatttacagaccggagagaaccatacctcagtggatccccgacaaactcaatttggaggtcttaacacaccctatccaggtggactaaacactccatacccaggtggaatgacgccaggactgatgacacctggcacaggtgagctggacatgaggaagattggccaagcgaggaacactctgatggacatgaggctgagccaggtgtctgactccgtgagtggacagaccgtcgttgaccccaaaggctacctgacggatttaaattccatgatcccgacacacggaggagacatcaatgatatcaagaaggcgcgactgctcctcaagtctgttcgggagacgaaccctcatcacccgccagcctggattgcatcagcccgcctggaagaagtcactgggaagctacaagtagctcggaaccttatcatgaaggggacggagatgtgccccaagagtgaagatgtctggctggaagcagccaggttgcagcctggggacacagccaaggccgtggtagcccaagctgtccgtcatctcccacagtctgtcaggatttacatcagagccgcagagctggaaacggacattcgtgcaaagaagcgggttcttcggaaagccctcgagcatgttccaaactcggttcgcttgtggaaagcagccgttgagctggaagaacctgaagatgctagaatcatgctgagccgagctgtggagtgctgccccaccagcgtggagctctggcttgctctggcaaggctggagacctatgaaaatgcccgcaaggtcttgaacaaggcgcgggagaacattcctacagaccgacatatctggatcacggctgctaagctggaggaagccaatgggaacacgcagatggtggagaagatcatcgaccgagccatcacctcgctgcgggccaacggtgtggagatcaaccgtgagcagtggatccaggatgccgaggaatgtgacagggctgggagtgtggccacctgccaggccgtcatgcgtgccgtgattgggattgggattgaggaggaagatcggaagcatacctggatggaggatgctgacagttgtgtagcccacaatgccctggagtgtgcacgagccatctacgcctacgccctgcaggtgttccccagcaagaagagtgtgtggctgcgcgccgcgtacttcgagaagaaccatggcactcgggagtccctggaagcactcctgcagagggctgtggcccactgccccaaagcagaggtgctgtggctcatgggcgccaagtccaagtggctggcaggggatgtgcctgcagcaaggagcatcctggccctggccttccaggccaaccccaacagtgaggagatctggctggcagccgtgaagctggagtccgagaatgatgagtacgagcgggcccggaggctgctggccaaggcgcggagcagtgcccccaccgcccgggtgttcatgaagtctgtgaagctggagtgggtgcaagacaacatcagggcagcccaagatctgtgcgaggaggccctgcggcactatgaggacttccccaagctgtggatgatgaaggggcagatcgaggagcagaaggagatgatggagaaggcgcgggaagcctataaccaggggttgaagaagtgtccccactccacacccctgtggcttttgctctctcggctggaggagaagattgggcagcttactcgagcacgggccattttggaaaagtctcgtctgaagaacccaaagaaccctgggctgtggttggagtccgtgcggctggagtaccgtgcggggctgaagaacatcgcaaatacactcatggccaaggcgctgcaggagtgccccaactccggtatcctgtggtctgaggccatcttcctcgaggcaaggccccagaggaggaccaagagcgtggatgccctgaagaagtgtgagcatgacccccatgtgctcctggccgtggccaagctgttttggagtcagcggaagatcaccaaggccagggagtggttccaccgcactgtgaagattgactcggacctgggggatgcctgggccttcttctacaagtttgagctgcagcatggcactgaggagcagcaggaggaggtgaggaagcgctgtgagagtgcagagcctcggcatggggagctgtggtgcgccgtgtccaaggacatcgccaactggcagaagaagatcggggacatccttaggctggtggccggccgcatcaagaacaccttctgattgagcggttgccatggccggtctccgtggggcagggttgggccgcatgtggaagggctctgagctgtgtcctccttcattaaaagtttttatgtctcgtgtcagaa

SEQ ID NO: 13 - DNA, 2940 bp, Homo sapiens
gccagctgtgccggcgtttgttggctgccctgcgcccggccctccagccagccttctgccggccccgccgcgatggaggtgccccagccggagcccgcgccaggctcggctctcagtccagcaggcgtgtgcggtggcgcccagcgtccgggccacctcccgggcctcctgctgggatctcatggcctcctggggtccccggtgcgggcggccgcttcctcgccggtcaccaccctcacccagaccatgcacgacctcgccgggctcggcagccgcagccgcctgacgcacctatccctgtctcgacgggcatccgaatcctccctgtcgtctgaatcctccgaatcttctgatgcaggtctctgcatggattcccccagccctatggacccccacatggcggagcagacgtttgaacaggccatccaggcagccagccggatcattcgaaacgagcagtttgccatcagacgcttccagtctatgccggtgaggctgctgggccacagccccgtgcttcggaacatcaccaactcccaggcgcccgacggccggaggaagagcgaggcgggcagtggagctgccagcagctctggggaagacaaggagaatgatggatttgtcttcaagatgccatggaagcccacacatcccagctccacccatgctctggcagagtgggccagccgcagggaagcctttgcccagagacccagctcggcccccgacctgatgtgtctcagtcctgaccggaagatggaagtggaggagctcagccccctggccctaggtcgcttctctctgacccctgcagagggggatactgaggaagatgatggatttgtggacatcctagagagtgacttaaaggatgatgatgcagttcccccaggcatggagagtctcattagtgccccactggtcaagaccttggaaaaggaagaggaaaaggacctcgtcatgtacagcaagtgccagcggctcttccgctctccgtccatgccctgcagcgtgatccggcccatcctcaagaggctggagcggccccaggacagggacacgcccgtgcagaataagcggaggcggagcgtgacccctcctgaggagcagcaggaggctgaggaacctaaagcccgcgtcctccgctcaaaatcactgtgtcacgatgagatcgagaacctcctggacagtgaccaccgagagctgattggagattactctaaggccttcctcctacagacagtagacggaaagcaccaagacctcaagtacatctcaccagaaacgatggtggccctattgacgggcaagttcagcaacatcgtggataagtttgtgattgtagactgcagatacccctatgaatatgaaggcgggcacatcaagactgcggtgaacttgcccctggaacgcgacgccgagagcttcctactgaagagccccatcgcgccctgtagcctggacaagagagtcatcctcattttccactgtgaattctcatctgagcgtgggccccgcatgtgccgtttcatcagggaacgagaccgtgctgtcaacgactaccccagcctctactaccctgagatgtatatcctgaaaggcggctacaaggagttcttccctcagcacccgaacttctgtgaaccccaggactaccggcccatgaaccacgaggccttcaaggatgagctaaagaccttccgcctcaagactcgcagctgggctggggagcggagccggcgggagctctgtagccggctgcaggaccagtgaggggcctgcgccagtcctgctacctcccttgcctttcgaggcctgaagccagctgccctatgggcctgccgggctgagggcctgctggaggcctcaggtgctgtccatgggaaagatggtgtggtgtcctgcctgtctgccccagcccagattcccctgtgtcatcccatcattttccatatcctggtgccccccacccctggaagagcccagtctgttgagttagttaagttgggttaataccagcttaaaggcagtattttgtgtcctccaggagcttcttgtttccttgttagggttaacccttcatcttcctgtgtcctgaaacgctcctttgtgtgtgtgtcagctgaggctggggagagccgtggtccctgaggatgggtcagagctaaactccttcctggcctgagagtcagctctctgccctgtgtacttcccgggccagggctgcccctaatctctgtaggaaccgtggtatgtctgccatgttgcccctttctcttttcccctttcctgtcccaccatacgagcacctccagcctgaacagaagctcttactctttcctatttcagtgttacctgtgtgcttggtctgtttgactttacgcccatctcaggacacttccgtagactgtttaggttcccctgtcaaatatcagttacccactcggtcccagttttgttgccccagaaagggatgttattatccttgggggctcccagggcaagggttaaggcctgaatcatgagcctgctggaagcccagcccctactgctgtgaaccctggggcctgactgctcagaacttgctgctgtcttgttgcggatggatggaaggttggatggatgggtggatggccgtggatggccgtggatgcgcagtgccttgcatacccaaaccaggtgggagcgttttgttgagcatgacacctgcagcaggaatatatgtgtgcctatttgtgtggacaaaaatatttacacttagggtttggagctattcaagaggaaatgtcacagaagcagctaaaccaaggactgagcaccctctggattctgaatctcaagatgggggcagggctgtgcttgaaggccctgctgagtcatctgttagggccttggttcaataaagcactgagcaagttgagaaaaaaaaaaaaaaaaaaa

SEQ ID NO: 14 - DNA, 2760 bp, Homo sapiens
aggagtctgtcagctacggaggacaatgaccttgcagacaccaccgcctgagtgagaaccaggggtctgtgcctctcctcattccccgctcttgcccttgtcaagcctgcaccagcatgtcaggaacctccaaggagagtctggggcatggggggctgccagggttgggcaagacctgcttaacaaccatggacacaaagctgaacatgctgaacgagaaggtggaccagctcctgcacttccaagaagatgtcacagagaagttgcagagcatgtgccgagacatgggccacctggagcggggcctgcacaggctggaggcctcccgggcaccgggcccgggcggggctgatggggttccccacattgacacccaggctgggtggcccgaggtcctggagctggtgagggccatgcagcaggatgcggcccagcacggtgccaggctggaggccctcttcaggatggtggctgcggtggacagggccatcgctttggtgggggccacgttccagaaatcaaaggtggcggatttcctcatgcaggggcgtgtgccctggaggagaggcagcccaggtgacagccctgaggagaataaagagcgagtggaagaagagggaggaaaaccaaagcatgtgctgagcaccagtgggttgcagtctgatgccagggagcctggggaagagagccagaaggcggacgtgctggaaaggacagcggagaggctgccccccatcagagcgtcagggctgggagctgaccccgcccaggcagtggtctcaccgggccagggagatggtgttcctggcccagcccaggcattccctggccacctgcccctgcccacaaaggtggaagccaaggctcctgagacacccagcgagaacctcaggactggcctggaattggctccagcacccggcagggtcaatgtggtctccccgagcctggaggttgcaccaggtgcaggacaaggagcatcgtccagcaggcctgaccctgagcccttagaggaaggcacgaggctgactccagggcctggccctcagtgcccagggcctccagggctgccagcccaggccagggcaacccacagtggtggagaaacacctccaaggatctccatccacatacaagagatggatactcctggggagatgctgatgacaggcaggggcagccttggacccaccctcaccacagaggctccagcagctgcccagccaggcaagcagggcccacctgggaccgggcgctgcctccaagcccctgggactgagcccggagaacagacccctgaaggagccagagagctctccccgctgcaggagagcagcagccccgggggagtgaaggcagaggaggagcaaagggctggggccgagcctggcacgagaccaagcttggccaggagtgacgacaatgaccacgaggttggggccctgggcctgcagcagggcaaaagcccaggggcgggaaaccctgagcctgagcaggactgtgcagccagggctccggtgagagctgaagcagtaaggaggatgcccccaggcgccgaggctggcagcgtggttctggatgacagtccggccccaccagctccttttgaacaccgggtagtgagcgtcaaggagacctccatctctgcgggttacgaggtgtgccagcacgaagtcttgggagggggtcggtttggccaggtccacaggtgcacagagaagtccacaggcctcccactggctgccaagatcatcaaagtgaagagcgccaaggaccgggaggacgtgaagaacgagatcaacatcatgaaccagctcagccacgtgaacctgatccagctctatgacgccttcgagagcaagcacagctgcacccttgtcatggagtacgtggacgggggtgagctcttcgaccggatcacagatgagaagtaccacctgactgagctggatgtggtcctgttcaccaggcagatctgtgagggtgtgcattacctgcaccagcactacatcctgcacctggacctcaagccggagaacatattgtgcgtcaatcagacaggacatcaaattaagatcattgactttgggctggccagaaggtacaagcctcgagagaagctgaaggtgaacttcggcactcctgagttcctggccccagaagtcgtcaattatgagtttgtctcattccccacagacatgtggagtgtgggagtcatcacctacatgctactcagtggcttgtccccatttctaggggaaacagatgcagagaccatgaatttcattgtaaactgtagctgggattttgatgctgacacctttgaagggctctcggaggaggccaaggactttgtttcccggttgctggtcaaagagaagagctgcagaatgagtgccacacagtgcctgaaacacgagtggctgaataatttgcctgccaaagcttcaagatccaaaactcgtctcaaatcccaactactgctgcagaaatacatagctcaaagaaaatggaagaaacatttctatgtggtgactgctgccaacaggttaaggaaatttccaacttctccctaatcttcaactctgctgctccaatgggtccagaaattactgaggccagtggtgaagtgaagagatgactcaaacatttaaataatttggcttttggtattattgattccacttatttgtaaatggttatggctgctgcttcctgtggatgaaagtggctgtaagagctcctagacgtttctgctgt

SEQ ID NO: 15 - DNA, 491 bp, Homo sapiens
gagccacacggcgcgacaagatggcggataaggagaagaagaaaaaggagagcatcttggacttgtccaagtacatcgacaagacgatccgggtaaagttccagggaggccgcgaagccagtggaatcctgaagggcttcgacccactcctcaaccttgtgctggacggcaccattgagtacatgcgagaccctgacgaccagtacaagctcacggaggacacccggcagctgggcctcgtggtgtgccggggcacgtccgtggtgctaatctgcccgcaggacggcatggaggccatccccaaccccttcatccagcagcaggacgcctagcctggccgggggcgcggggggtgcagggcaggcccgagcagctcggtttcccgcggacttggctgctgctcccaccgcagtaccgcctcctggaacggaagcattttcctttttgtataggttgaatttttgttttcttaataaaattgcaaacctcaa

SEQ ID NO: 16 - DNA, 4376 bp, Homo sapiens
aacagaagatgccggagaaggggggggaaaagtagatgcggatttcgtcctgacttctaaaaattcctcctctccctctcccattttcctaatccgagaatgatggagctcgaggcaaaggaatgattccggaaatggagatatgattctcaaacctagaaatgatcggagtgatttattagttaaatattcttcgtccaggaacccagcacaattcagagctgcagattggatattgggaagcaaatttgggtgtgaaatcttcagcaaaggagcacgcagagtccatgatggctcagaccaagtgagtgagaggcagagcgaggacgcccctctgctctggcgcgcccggactcggactcgcagactcgcgctggctccagtctctccacgattctctctcccagacttttccccggtcttaagagatcctgtgtccagagggggccttagctgctccagcccgcgatgaggaaaagtccaggtctgtctgactgtctttgggcctggatcctccttctgagcacactgactggaagaagctatggacagccgtcattacaagatgaacttaaagacaataccactgtcttcaccaggattttggacagactcctagatggttatgacaatcgcctgagaccaggattgggagagcgtgtaaccgaagtgaagactgatatcttcgtcaccagtttcggacccgtttcagaccatgatatggaatatacaatagatgtatttttccgtcaaagctggaaggatgaaaggttaaaatttaaaggacctatgacagtcctccggttaaataacctaatggcaagtaaaatctggactccggacacatttttccacaatggaaagaagtcagtggcccacaacatgaccatgcccaacaaactcctgcggatcacagaggatggcaccttgctgtacaccatgaggctgacagtgagagctgaatgtccgatgcatttggaggacttccctatggatgcccatgcttgcccactaaaatttggaagttatgcttatacaagagcagaagttgtttatgaatggaccagagagccagcacgctcagtggttgtagcagaagatggatcacgtctaaaccagtatgaccttcttggacaaacagtagactctggaattgtccagtcaagtacaggagaatatgttgttatgaccactcatttccacttgaagagaaagattggctactttgttattcaaacatacctgccatgcataatgacagtgattctctcacaagtctccttctggctcaacagagagtctgtaccagcaagaactgtctttggagtaacaactgtgctcaccatgacaacattgagcatcagtgccagaaactccctccctaaggtggcttatgcaacagctatggattggtttattgccgtgtgctatgcctttgtgttctcagctctgattgagtttgccacagtaaactatttcactaagagaggttatgcatgggatggcaaaagtgtggttccagaaaagccaaagaaagtaaaggatcctcttattaagaaaaacaacacttacgctccaacagcaaccagctacacccctaatttggccaggggcgacccgggcttagccaccattgctaaaagtgcaaccatagaacctaaagaggtcaagcccgaaacaaaaccaccagaacccaagaaaacctttaacagtgtcagcaaaattgaccgactgtcaagaatagccttcccgctgctatttggaatctttaacttagtctactgggctacgtatttaaacagagagcctcagctaaaagcccccacaccacatcaatagatcttttactcacattctgttgttcagtcctctgcactgggaatttatttatgttctcaacgcagtaattcccatctgctttattgcctctgtcttaaagaatttgaaagtttccttattttcataattcatttaagaacaagagacccctgtctggcagtctggagcaaagcagactatgcagcttggagacaggattctgacagagcaagcgaaagagcaaagtcatgtcagaaggagacagaatgagagagaaaagagggggaagatggttcaaagatacaagaaaaagtagaaaaaaaaataacacttaactaaaacccctaggtcatttgtagatatatatttccaaatattctaaaaaagatactgtatatgtcaaaaatatttttatgtgaaggtgtttcaaagggtaaattataaatgtttcatgaagaaaaaattttaaaaatctacgtctttattacacaaactatggtgtgcttatgtttttgttttgctttttaaactgatgtatagctttaacattttgtttccaaagctgaagatccccattctttctctttgaaaaaaaaaaaggcctaatgcattattttgtcataaaatgctattttaaaattcatggaactttcatacgtaaaggtgcagttgctcattgtagagcacatttagtccaatgaagataaatgctttaaatagtttacttcactttcatctgagcttttaccactagactcaaggaagaataattttaacagacatgtatactccatagaaactaaactaaaatagtttaaaaatattccctttttcaccctattttcagatagcacatgagcccaacactcacttaattctcattatgaagatgtttttagaggggcaaaaatattttgcaagctctggaattgttgaatgtattcttttatataactacattaaaagctttagattgaaatttatgactagcaaacaaaaatagaatatataaacgatatatgtaaatatacagcatgagattgtacattttttacttttttaaaattgtgttcttaaaatattgtgtaagaatcactgcacttagctgttggaatgttgttaaatgctatggaaatacatttagaacctgcatttaagaacagaacagcaagtatgaaccacatggaacttaaaacatatgggtgtgaagtccacttatgtagacaaaacttataatttccaaactgttgtctagtatacagtgatcagttgctctctgttcaagtcattccacacatttccctattttaggctattataatatagaaagaaaatgggaagcattagttggagctagaaaatgaactgtatattattgctatatttgctaataccaactatttcaataagtgttgtaccatatgtagcattaaatataaaatacataaaagaatgtacagaaaatagcttttattgagtaatattacatttcatttatactgtagcaatatatttgtaggtatactatgtaagggctttaaataaaagaggtccattaatacttccttataaaaattctagtctgtttcattactgcccagatgttttagagataaatatttatgcagaaggtatttttgaagtctccttttgtctgatagagtttaacagatatttaaatttagtgctcagaatccacaagtcacggtctaaacacacttagaatactacagcataaatctgttagcattattgccaaataagacagttgggatccaaacccaagtcttgagcaatgtttttctcaaaaagctgctatccaatgatataggaaaatacattgtgttttcctaaacacacttttctttttaaatgtgcttcattgtttgatttggtcctgcctaaatttcacaagctaggccaatgaaggctgaatcaaagacatttcatccaccaatatcatgtgtagatattatgtatagaaaataaaataaattatggctctaacttctgtgttgctgtttatcttgttatttttcggcgttatactaatgtgtttattgagagcattttaccttccagacttctcatggctaacttttggtctgtattttgctccttagatgtgaatatttcttattagtctgcttcctgctacgcaatgactgcatttctatcatttctcagtttgttagtatatgtggatagtattctactgtataaatgattgcaaagtttatcaaaaacaaattattatatgtagcttttctacagtgctttgctaaaccatgtagtactagttaagtcttccttgaaaataaagatacactcttataggggacagttcctgttcactcccaggaaacttttttaaaagatgacactgaatgtttattgcactttagtgcagtgaagtggcaataaaacctaacatgaatcaaggttgtttatggcagatgcatgtgttgctttacagagtttagcaaaagctcttaattttatgtcatactgtattctactgaataataaagctaacattattcaataataaaatggaa

SEQ ID NO: 17 - DNA, 3309 bp, Homo sapiens
agacaacacagaaaccgagaaacgcgcggaaatactggagtttttcgactctgagggcagtaaaggatccgttaatttctgtttgttttcgacacatgaacgcgcgggttattcacaacacactttcaacacattctcaacgcaaagtgaagagaaatgggcaaaaagaagcaggaggaagaacaaaatagctccgagtacggagctccagctcagtatgacccgacattcaatggccctattcacaaaaggagctgtacagacatcatctgctgtgtgctcttcatgctggtcatcactgggtacatggtggtggggattctggcctggttgtatggagaccctcggcatgttctgtatcctagaaactccacaggaatgttctgcgggatcggccagaaccagaataaacccagcgtcctgtacttcgacatcctcaaatgtgcgacggccacaaacatcatggcagcggctctacaaggccttcagtgtcccacaactcaggtgtgtgtgtcgtcatgtccgtcaggattctgggctttatctcctctggcgtatctgcccaacgcaaaaccagccgattatttccagcaggagctctgtgtgccgtcactccagctcaaagacaccacttatacggtgatggagatcatcaataaagagttgtgtccttattactacacacctacaacatcagtgttggatcgctgtttgcccagtttaggtggaagcgcttataaccccagtaacattcctgcaaacttcagtcttccagggctgagtataaaccagacattgtcgacgatcgccaatgctacaagtgatctgaccaacagcttcaacatgaaagacgtgggtctgcgcatctttgaggacttcgctaagacctggcagtggatcgtggctggtctggtgatagcgatggtggtcagtgtgctgttcctcctgctgctgcggtttacagctccggtgctcatctggatcctcatcttcggtgttctggccgttggagcattcgggatctggtactgctataatgactacatgtctctggcgagctccaacctgaccttcagtaatgtgggcttcaccactaatgttcaggtgtatctgcaggtgcgagacacatggctggccttcttgatcattctgtgtatcgttgaggctgttctgattctggctttgatcttcttgaggacccgaatcctcatcgccatcgctctcattcaggagaccagcaaggctttgggtcacatgatgtcgactctcctctatcccgtggtgacgtttgtgctgctgttggtgtgtgtgtcgtactggggaatcacagcgctatatctggccacatcaggtgctcccatttataaagttgtggctctgaacacgacacagggagactgcagcaacatacaagccaaccagacctgcgaccctcagacattcaacagctctcggtactcctcatgtccgtccgcgcgctgcgtctttatcaactataacaccgagggcttgttccagaggaatctcttcaatctgcagatctataacgttgtggcgtttctgtggtgtgtgaacttcgtcatcgctctcggtcactgcacgctcgccggagccttcgcttcctattactgggccttcagtaaacctgcagatatcccaacattccctctgactcagtccttcatgagggccctcaggtatcatgtgggttcgctggcgttcggcgctctgattctcacactcgtgcagatcgtcaggatcatcctggagtatctggaccacaagttcaaagcggctcaaaacccgtgcgcacgcttcctcatgtgttgcctgaaatgctgcttctggtgtctggaaaaattcatcaagttcatcaacagaaacgcgtacatcatgatcgccatatacggaaaaaacttctgcgtgtcggctaaaaacgcctttttcctgctgatgaggaacatcgtacgggtggtggttctggataaagtgacagatctgcttctgtttttcgggaagctgctggttgttggtggaatcggagttctggccttcttcttcttctccggcaggattcagcttccaggaaacacgtttcagaccgcggcgctcaactattactggatgcccatcatcacggtggtgtttggagcgtatctgatcgctcatggcttcttcagtgtctataacatgggtgtagatacactgttcctgtgcttcttggaggatctggagagaaacgacggcagcgcagagaaaccctacttcatgtccaaaaacctgatgaagatcctcaacaagaagaacaaacagcccaagacgggctaacatgatgagcctttactctctcgagcaccgtcactttgacacgacgcatgcagatgcttctagagacacttttaaacctgcattattactgttttaatagatgttactgagagcttctagttcaacacacactccattctcaatctccaccaaagacgccgaatcacagccaaacccatgagaagcgatgttcctactcaacatgtgccagtttttagtttaatatgtgcacttttaacttcttttcaacagaaatgcctgacaagagctggagtagtgccttcagcgtgacgtctgtgagccaaagcgtttatcagcactcaatattaatgcagtttttagtgctgtgcacaaataaatcacacgttttaaatgaaaagtttctgacattacatgtgtgtgtgtgtttattatgtgtatgtgatgcacacatgcatgcatatatctgagaacatgtttatttatatttagagacaacatttctatgtatatgatgcagattagatagcattttaaatatctgtgtgaccagattgttattatgcatgtgtgtgtgtgtgtcagatatacatatataacacacacacatatcgcttatgtcaaaacacacgtttattttagatgtgcttaaactttacccagtgctactttagtttgtggatgttttagacatgcatattgcacatgtgattattaatgcagaataataagcagctgataatctgttcattaaatcacggtactgttttaaagcccgctctgtgcaacagaaacgtctgacttatggaacgtcacatgcgtgagcaattctgtttttacattttaaacagctttatgtgattgtttatgaagtgatttagttgattctggaaatcgatatttatttaaagacagaactatatgtgttaataaaatgaggagaatgaaaaaaaaaaaaaaaaaaaaaaaaaaaaaaaaaaaaaaaaaaaaaaaaaaaaaaaa

SEQ ID NO: 18 - DNA, 799 bp, Homo sapiens
ggtcttcttccttctcgcctaacgccgccaacatggtgttcaggcgcttcgtggaggttggccgggtggcctatgtctcctttggacctcatgccggaaaattggtcgcgattgtagatgttattgatcagaacagggctttggtcgatggaccttgcactcaagtgaggagacaggccatgcctttcaagtgcatgcagctcactgatttcatcctcaagtttccgcacagtgcccaccagaagtatgtccgacaagcctggcagaaggcagacatcaatacaaaatgggcagccacacgatgggccaagaagattgaagccagagaaaggaaagccaagatgacagattttgatcgttttaaagttatgaaggcaaagaaaatgaggaacagaataatcaagaatgaagttaagaagcttcaaaaggcagctctcctgaaagcttctcccaaaaaagcacctggtactaagggtactgctgctgctgctgctgctgctgctgctgctgctgctgctaaagttccagcaaaaaagatcaccgccgcgagtaaaaaggctccagcccagaaggttcctgcccagaaagccacaggccagaaagcagcgcctgctccaaaagctcagaagggtcaaaaagctccagcccagaaagcacctgctccaaaggcatctggcaagaaagcataagtggcaatcataaaaagtaataaaggttctttttgacctgttgaaaaaaaaaaaaaaaaaaaaaaaaaaaaaaaaaaaaaaaaaaaaaaaaaaaaaaaaaaaaaaaaaaa

SEQ ID NO: 19 - DNA, 1336 bp, Homo sapiens
actgcgcaggcgcttacagtgcaccaagatggccgcccccgtggatctagagctgaagaaggccttcacagagcttcaagccaaagttattgacactcaacagaaggtgaagctcgcagacatacagattgaacagctaaacagaacgaaaaagcatgcacatcttacagatacagagatcatgactttggtagatgagactaacatgtatgaaggtgtaggaagaatgtttattcttcagtccaaggaagcaattcacagtcagctgttagagaagcagaaaatagcagaagaaaaaattaaagaactagaacagaaaaagtcctacctggagcgaagcgttaaggaagctgaggacaacatccgggagatgctgatggcacgaagggcccagtagggagcctctctgggaagctcttcctcctgcccctcccattcctggtgggggcagaggagtgtctgcagggaaacagcttctcctctgccccgatggatgctttatttggatggcctggcaacatcacattttctgcatcaccctgagccccatttgcttcccagccctggagtttttacccggctttgcctgccacctctgcccaggacactcttccctctcgggatgtgtgatgaactcccaggagagggaagatgggagccagggcaagataggaagctctgcctgagctttccactaggcacgccagccagaccaataaaaagcgtctgtcccactctgctaagcctggttttcttgagcagagggatggaacagagggtgagagaggcagtggccgtctccacctcagctcctgctccctctgcatcagagcccttcctttcttgggggatgggccttgccctcttctcttttcccttcctgtacctttgactaacgctcagcttccgggcctgcatgcagtagacagaagaggaagaaagaacagatgttcacagctgaatctcagtgaacagaatagcagtccctggatggcagtctgcctaaagattcctttccctgccttctcccatacattccaaaaggaagttcaacagtaagcagcacctccaagactgtctccttttggccaatatcataagatggacgccataatcctgaggcctcctagaggctgagggggcaacggtgtgatccagctggctcatcccagcccaggtgggccaattattcaattttcaagaattttgttgcaagccagttgtcaaacacagccattataattatgtaaatttgcaaattatgttaaaaacaaggacaataaatattcaaaatgcatccctaattactacatttgaccacgatctgtgctctatc

SEQ ID NO: 20 - DNA, 2376 bp, Homo sapiens
cttctccgcctccgcctcctcccgacgccggcgccgctttctggaaggttcgtgaaggcagtgagggcttaccgttattacactgcggccggccagaatccgggtccatccgtccttcccgagccaacccagacacagcggagtttgccatgcccgagaatgtggcaccccggagcggggcgactgccggggctgccggcggccgcgggaaaggcgcctatcaggaccgcgacaagccagcccagatccgcttcagcaacatttccgccgccaaagcggttgctgatgctattagaacaagccttggaccaaaaggaatggataaaatgattcaagatggaaaaggtgatgtaaccattacaaatgatggtgctaccattctgaaacaaatgcaagtattacatccagcagccagaatgctggtggagctgtctaaggctcaagatatagaagcaggagatggcaccacatcagtagtcatcattgctggctccctcttagattcttgtaccaagcttcttcagaaagggattcatccaaccatcatttctgagtcattccagaaggccctggaaaagggcattgaaatcttgactgacatgtctcgacctgtggaactgagtgacagagaaactttgttaaatagtgcaaccacttcactgaactcaaaggtggtttctcagtattcaagtctgctttctccaatgagtgtaaatgcagtgatgaaagtgattgacccagccacagccaccagtgtagatcttagagatattaaaatagttaagaagcttggtgggacaattgatgactgtgagttggtggaagggctggttctcacccaaaaagtgtcaaattctggcataaccagagttgaaaaggccaagattgggcttattcagttttgcttatctgctcccaaaacagacatggataatcaaatagtggtttctgactatgcccagatggaccgagtgctgcgagaagagagagcctatattttaaatttagtgaagcaaattaaaaaaacaggatgtaatgtccttctcatacagaaatctattctaagagatgctcttagtgatcttgcattacactttctgaataaaatgaagatcatggtgattaaggatattgaaagagaagacattgaattcatttgtaagacaattggaaccaagccagttgctcatattgaccaatttactgctgacatgctgggttctgctgagttagctgaggaggtcaatttaaatggttctggcaaactgctcaagattacaggctgtgccagccctggaaaaacagttacaattgttgttcgtggttctaacaaactggtgattgaagaagctgagcgctccattcatgatgccctatgtgttattcgttgtttagtgaagaagagggctcttattgcaggaggtggtgctccagaaatagagttggccctacgattaactgaatattcacgaacactgagtggtatggaatcctactgcgttcgtgcttttgcagatgctatggaggtcattccatctacactagctgaaaatgccggcctgaatcccatttctacagtaacagaactaagaaaccggcatgcccagggagaaaaaactgcaggcattaatgtccgaaagggtggtatttccaacattttggaggaactggttgtccagcctctgttggtatcagtcagtgctctgactcttgcaactgaaactgttcggagcattctgaaaatagatgatgtggtaaacactcgataatctggataactgactagcaccattatgatcaccagtattgtggctggaatggaagaagatcaccttggtgttccttgtttggaagattatttcctctgaatttctgggcttggtcttccagttggcatttgcctgaagttgtattgaaacaatttaatgaaaatattaaatatttggtttcaaaaggcagatttatcttctcccaacattctgttatttctgatacttttgaaaaactaataaaaactaataaaagaagcgtaaaaagtgagtttacatgttgaggaaaaaaatggcccaatatgctcatcactgataaatgctccctggccttaaaaactaccaacatataatatatatgctgtcttaaaagttaatgatccaagtggcacctctctgaacctactttggcttgggaggctgcccagttaaaacaaaaataagttaatggtacagaaagagaatgaaaaatgaaagcctccttttatcctatcatcctaattccttttcccagtaataatgactgctgtgttggattccttctgcaaataaaagtgtatacatatatgtagcaaatcttacttaaacaaaagggtttcttaactaa

SEQ ID NO: 21 - DNA, 1005 bp, Homo sapiens
agagaggctgagaccaacccagaaaccaccacctctcacgccaaagctcacaccttcagcctccaacatgaaggtctccgcagcacttctgtggctgctgctcatagcagctgccttcagcccccaggggctcgctgggccagcttctgtcccaaccacctgctgctttaacctggccaataggaagataccccttcagcgactagagagctacaggagaatcaccagtggcaaatgtccccagaaagctgtgatcttcaagaccaaactggccaaggatatctgtgccgaccccaagaagaagtgggtgcaggattccatgaagtatctggaccaaaaatctccaactccaaagccataaataatcaccatttttgaaaccaaaccagagcctgagtgttgcctaatttgttttcccttcttacaatgcattctgaggtaacctcattatcagtccaaagggcatgggttttattatatatatatatttttttttttaaaaaaaaaacgtattgcatttaatttattgaggctttaaaacttatcctccatgaatatcagttatttttaaactgtaaagctttgtgcagattctttaccccctgggagccccaattcgatcccctgtcacgtgtgggcaatgttccccctctcctctcttcctccctggaatcttgtaaaggtcctggcaaagatgatcagtatgaaaatgtcattgttcttgtgaacccaaagtgtgactcattaaatggaagtaaatgttgttttaggaatacataaagtatgtgcatattttattatagtcactagttgtaatttttttgtgggaaatccacactgagctgagggggacaaagatggctgtggccaagaggggcttggttaagggggtgggaactatgtccctgggaaatgagtttttggcttagctggtcttcattgaaatgcagggtgaaactgacaaacccattccagccctctattcccattttcaacagtatttcc

(19) United States
(12) Patent Application Publication
(10) Pub. No.: US 2022/0290243 A1
(43) Pub. Date: Sep. 15, 2022

(54) IDENTIFICATION OF PATIENTS THAT WILL RESPOND TO CHEMOTHERAPY

(71) Applicant: Rutgers, The State University of New Jersey, New Brunswick, NJ (US)

(72) Inventors: Antonina Mitrofanova, New York, NY (US); Nusrat J. Epsi, Newark, NJ (US)

(73) Assignee: Rutgers, The State University of New Jersey, New Brunswick, NJ (US)

(21) Appl. No.: 17/624,027

(22) PCT Filed: Jun. 30, 2020
(86) PCT No.: PCT/US2020/040333

§ 371 Date: Dec. 30, 2021

Related U.S. Application Data
(60) Provisional application No. 62/869,499, filed on Jul. 1, 2019.

(51) Int. Cl. C12Q 1/6886; G16B 20/00

(52) U.S. Cl. CPC C12Q 1/6886; G16B 20/00; C12Q 2600/106; C12Q 2600/154; C12Q 2600/156

(57) ABSTRACT

Disclosed herein are methods of treating and identifying subjects with cancer that will respond to chemotherapy treatment. Exemplary methods can be used to treat or identify subjects with lung or colorectal cancer that will respond positively (or will not respond) to chemotherapy.

### CROSS-REFERENCE TO RELATED APPLICATION

This application claims priority to U.S. Provisional Application No. 62/869,499, filed Jul. 1, 2019, herein incorporated by reference in its entirety.

### FIELD

This disclosure relates to methods of treating and identifying subjects with cancer, such as a lung or colorectal cancer, which will respond to chemotherapy treatment, for example subjects that will not become resistant to the chemotherapy treatment.

### BACKGROUND

Lung adenocarcinoma (LUAD) is a major cause of cancer-related death in the United States with a five-year survival rate of 17.7% (Siegel et al., Cancer Statistics. 2017; 67(1):7-30.). The majority of patients with LUAD lack “clinically actionable” mutations and are commonly treated with a platinum-based doublet chemotherapy (i.e., often combined with plant alkaloids and/or antimetabolites) to improve response rates and survival (Anderson et al., Br. J. Canc. 2000; 83(4):447-53, Pfister et al., J. Clin. Oncol. 2004; 22(2):330-53, Lilenbaum et al., J. Clin. Oncol. 2005; 23(1):190-6., Lilenbaum et al., J. Thor. Oncol. 2009; 4(7):869-74, Zhu et al., J. Clin. Oncol. 2010; 28(29):4417-24, Tang et al., Clinical cancer research 2013; 19(6):1577-86, Soo et al., J. Thor. Oncol. 2017; 12(8):1183-209, Hirsch et al., Lancet. 2017; 389(10066):299-311). Treatment for LUAD includes administration of immune checkpoint inhibitors, but they are not curative for most patients (Hirsch et al., Lancet. 2017; 389(10066):299-311). The heterogeneity of response to standard-of-care therapies and emerging treatment resistance remain major challenges in lung cancer management. Prioritization of patients based on their risk of developing resistance prior to therapy administration would significantly improve disease course and enhance informed clinical decision making at large.

### SUMMARY

Identification of patients with a poor or positive (i.e., favorable) chemotherapy response prior to treatment administration remains a major challenge in clinical oncology and cancer management. Methods of treating a subject with cancer (such as lung or colorectal cancer) and/or identifying subjects with cancer that will positively respond to chemotherapy treatment (or will likely not respond to such treatment) are disclosed herein. In some examples, the methods include measuring expression and/or methylation of cancer-related molecules from cancer-related pathways in a sample obtained from a subject. Cancer-related pathways (such as lung or colorectal cancer-related pathways) can include chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, calcium signaling, RNA degradation, neuroactive ligand-receptor interaction, SLC-mediated transmembrane transport, and translation pathways. In some examples, the cancer-related molecules include CCL22, CCR9, POLR2C, FGFR1OP, CCT4, PDE7A, DTYMK, ARPC1A, RPLP2, CCL11, ERCC1, U2AF1, SF3B3, PRPF6, CDC25B, MYLK3, LSM7, GABRA1, SLC44A4, RPL14, and/or PFDN1.

The methods can include identifying a subject with cancer who will not respond positively to chemotherapy treatment, for example, where expression of the cancer-related molecules differs from a control representing expression for the cancer-related molecules expected in a sample from a subject who positively responds to a chemotherapy and/or wherein methylation of the cancer-related molecules differs from a control representing methylation for the lung cancer-related molecules expected in a sample from a subject who positively responds to a chemotherapy. In some examples, the methods include administering at least one of surgery, radiation therapy, targeted therapy, immunotherapy, or palliative care to a subject with cancer (such as lung cancer) who is identified as one who will not respond positively to chemotherapy, thereby treating the subject.

The methods can include identifying a subject with cancer who will respond positively to a chemotherapy, for example, where expression of the cancer-related molecules is similar to a control representing expression for the cancer-related molecules expected in a sample from a subject who positively responds to a chemotherapy and/or methylation of the cancer-related molecules is similar to a control representing methylation for the cancer-related molecules expected in a sample from a subject who positively responds to a chemotherapy. In some examples, the methods include administering a chemotherapy to a subject with cancer who is identified as one who will respond positively to chemotherapy, thereby treating the subject,

In some examples, a subject who will develop resistance to chemotherapy is one who will have a recurrence of their cancer within one year of treatment with the chemotherapy. In some examples, a subject who will not develop resistance to chemotherapy is one who will not have a recurrence of their cancer within one year of treatment with the chemotherapy.

The foregoing and other objects and features of the disclosure will become more apparent from the following detailed description, which proceeds with reference to the accompanying figures.

### BRIEF DESCRIPTION OF THE DRAWINGS

FIG. 1 shows a schematic representation of an example pathway altered at both genomic and epigenomic levels. Pathway genes affected on genomic and epigenomic levels in G alpha signaling events pathway are represented by ovals, and the colors correspond to either over-expression (red), under-expression (blue), or no differential expression (white). Small satellite circles represent over-methylation (red) or under-methylation (blue).

FIGS. 2A-2D show an example integrative systematic epigenomic analysis that identifies candidate molecular pathways for a chemotherapy response. FIG. 2A shows an example schematic representation of the integrative epigenomic analysis. From left to right, (left) patients are defined by their response to chemotherapy, (middle left) analysis of genomic and epigenomic patient profiles, (middle right) integrative epigenomic analysis identifies candidate pathways affected on both genomic and epigenomic levels, and (right) multi-modal validation of candidate pathways.

FIG. 2B shows an example box and whisker plot depicting p-value cutoff for query carboplatin-paclitaxel response composite methylation pathway signature (x-axis) and NESs from the corresponding GSEA comparison between composite methylation and expression pathways signatures (y-axis), based on analysis in TCGA-LUAD patient cohort. The arrow indicates an optimal p-value threshold, which results in the most significant GSEA enrichment. FIG. 2C shows an example GSEA comparing a carboplatin-paclitaxel response composite expression pathway signature (reference) and carboplatin-paclitaxel response composite methylation pathway signature (query, p<0.001) based on the analysis in the TCGA-LUAD patient cohort. The horizontal red bar in the top left corner indicates leading edge pathways that are altered on both genomic and epigenomic levels. The NES and p-value were estimated using 1,000 pathway permutations. FIG. 2D shows an example ROC analysis comparing ability of the 7 candidate pathways to predict carboplatin-paclitaxel where their activity is defined based on their expression values (green) or methylation values (blue). The AUROC is indicated.

FIGS. 3A-3B show epigenomic alterations in candidate molecular pathways of carboplatin-paclitaxel response. FIG. 3A shows representative molecular pathways altered on both genomic and epigenomic levels, visualized through circlize (Gu et al., Bioinformatics. 2014; 30(19):2811-2) R package. Genes from the leading edge in each pathway are represented as differentially expressed (pink), methylated (grey), and both differentially expressed and methylated (yellow). The width of each connecting line is proportional to the extent of differential expression and differential methylation. From left to right, (left) chemokine receptors bind chemokines pathway (19 differentially expressed genes, 4 differentially methylated genes, and 8 differentially expressed and methylated genes), (middle) mRNA splicing pathway (21 differentially expressed genes, 39 differentially methylated genes, and 28 differentially expressed and methylated genes), and (right) G alpha signaling events pathway (37 differentially expressed genes, 8 differentially methylated genes, and 4 differentially expressed and methylated genes). FIG. 3B shows a 7-candidate pathway network representation, in which nodes correspond to the genes, which are connected to central pathway-membership circles (i.e., indicating pathway membership). Gene colors describe differential expression (pink), differential methylation (grey), and both differential expression and methylation (yellow). An example network was constructed using igraph (Csardi et al., InterJournal, Complex Systems. 2006; 1695(5):1-9), sna (Butts C T., Social Network Analysis with sna. 2008. 2008; 24(6):51), ggplot2 (Wickham, J Stat Softw. 2010; 35(1):65-88) and ggnetwork (Briatte F. Ggnetwork: Geometries to Plot Networks with ‘ggplot2’2016(R package version 0.5.1.) R packages. A list of genes in each pathway is shown below.

Carb + Pac LUAD

| pathway | changes | genes |
|---|---|---|
| intestinal immune network | both | IL4, IL2, HLA-DOB, TNFRSF13B, PIGR |
| for IgA production | | |
| intestinal immune network | epigenomic | CD80, CD28, CD86, IL5, ICOS, IL10, TNFRSF17 |
| for IgA production | | |
| intestinal immune network | transcriptomic | IL6, HLA-DMA, HLA-DQA2, HLA-DRB5, HLA- |
| for IgA production | | DRA, TGFB1, TNFSF13, HLA-DQB1, HLA-DRB1, |
| | | MAP3K14, HLA-DPA1, ICOSLG, HLA-DQA1, |
| | | HLA-DPB1, CD40LG, HLA-DOA, AICDA |
| RNA degradation | both | HSPD1, LSM5, XRN2, LSM3, PAPOLG, ENO1, |
| | | MPHOSPH6, LSM2, ZCCHC7, EXOSC9, PNPT1, |
| | | C1D, WDR61 |
| RNA degradation | epigenomic | TTC37, DIS3, LSM1, LSM4, PAPOLA, ENO3, |
| | | CNOT3, DCP1A, CNOT8, CNOT4, XRN1, CNOT7, |
| | | POLS, HSPA9, CNOT10, LSM6, EXOSC7, CNOT2, |
| | | EDC4, EXOSC4, LSM7 |
| RNA degradation | transcriptomic | EXOSC10, EXOSC5, EXOSC2, EDC3, RQCD1, |
| | | EXOSC3, EXOSC8 |
| cell cycle mitotic | both | FEN1, MCM4, PSMB4, E2F5, ANAPC7, CENPM, |
| | | PSMD12, CCNB2, CENPJ, KIF2C, UBB, PSMD9, |
| | | ANAPC5, TUBGCP5, CDC23, MNAT1, BUB1B, |
| | | CDCA8, MCM3, PSMD4, BUB1, PLK4, AURKA, |
| | | TUBB, PSMD14, PSME3, RFC5, CENPE, PSMD7, |
| | | XPO1, PSMB5, CDC25A, RFC4, FBXO5, MCM6, |
| | | BIRC5, CCNE2, PRIM1, CDK4, NUP107, CENPO, |
| | | PSMB6, PSMA3, PPP2R5C, CENPL, POLA2, |
| | | ZW10, GINS4, PRIM2, NUP160, NUF2, SKP2, |
| | | CDC25C, E2F2, PLK1, MAPRE1, PSMD6, DSN1, |
| | | DYNLL1, ANAPC1, PSMB3, MAD2L1, RAD21, |
| | | PSMB1, NEK2, ANAPC11, KIF23, PSMC6, E2F4, |
| | | PSMA6, PSMA5, DCTN2, RPA2, CASC5, CENPN, |
| | | ITGB3BP, MIS12, CEP57, E2F3, CDC20, SGOL1, |
| | | RFC2, PPP2R5D, MCM2, POLD3, NEDD1, |
| | | PSMB7, CENPK, ZWILCH, ZWINT, PSMC4, |
| | | POLD2, PSMD8, PSMC2, MCM8, CDC7, E2F1, |
| | | FGFR1OP, AURKB |
| cell cycle mitotic | epigenomic | TUBB2C, PPP2R5A, TUBGCP6, PTTG1, SMC3, |
| | | PSMC3, CDK7, PPP2R5B, CSNK1E, TUBGCP2, |
| | | PSMB9, ORC6L, RANGAP1, PCM1, CEP72, |
| | | CDC14A, CCNH, AKAP9, CSNK1D, MLF1IP, |
| | | PPP2R1B, CDK5RAP2, TUBGCP3, NDEL1, |
| | | PAFAH1B1, STAG1, ORC3L, CEP63, CLIP1, |
| | | BTRC, CP110, PPP2R1A, CDK6, UBE2D1, |
| | | PSME2, SEC13, TK2, CDKN1A, WEE1, CLASP2, |
| | | CENPF, SFI1, ACTR1A, PPP2CB, CEP70, |
| | | CCDC99, PRKAR2B, CDC25B, PSMB10, cell cycle |
| | | mitotic 2, PRKACA, RFC1, NDE1, PPP2CA, |
| | | CKAP5, POLE, ANAPC4, NUDC, SKP1, ORC1L, |
| | | ORC5L, ORC2L, ORC4L, CENPT |
| cell cycle mitotic | transcriptomic | UBE2E1, KIF2A, RB1, STAG2, POLA1, KIF18A, |
| | | PSMB2, BUB3, PSMD2, TUBGCP4, KIF20A, |
| | | CEP78, CDC26, PSMA4, PKMYT1, PPP1CC, |
| | | CCNB1, CEP152, YWHAG, NDC80, APITD1, |
| | | CENPH, HSP90AA1, PSMB8, RPA1, DYNC1I2, |
| | | TUBG1, PSMD11, POLD1, NUP133, MCM5, |
| | | TYMS, TFDP1, POLE2, INCENP, CDT1, Ecell |
| | | cycle mitotic 6L, NUP43, CENPI, CCNA2, SGOL2, |
| | | CENPA, MCM7, POLD4, NUP85, NUP37, |
| | | PSMD10, MCM10, YWHAE, PSMA2, DBF4, LIG1, |
| | | PMF1, GINS2, DCTN3, SPC25, DNA2, NSL1, |
| | | RPA3, GINS1, RRM2, ANAPC10, PSMD3, PSMC1, |
| | | SPC24, CKS1B, CDC6, CEP76, CENPP, CENPQ, |
| | | CDK2, PSMA7, PCNA, GMNN, RFC3 |
| chemokine receptors bind | both | XCR1, CXCL6, CXCL5, CCL19, CXCR5, PPBP, |
| chemokines | | CCR9, CCL22 |
| chemokine receptors bind | epigenomic | CCL28, CCR4, CCR10, CCR8 |
| chemokines | | |
| chemokine receptors bind | transcriptomic | CCL20, CXCL3, CCL16, CCL2, CXCL16, CCL5, |
| chemokines | | CXCL1, CCL3L3, CXCL12, CXCL13, CX3CR1, |
| | | CXCL2, CX3CL1, CCL21, CXCR4, CCL27, CCR7, |
| | | CCR6, CCL17 |
| G alpha (s) signalling | both | RLN3, PDE4C, PTGIR, GNB4 |
| events | | |
| G alpha (s) signalling | epigenomic | CALCB, ADCY4, LHB, HTR6, ADCY7, GNB5, |
| events | | MC4R, HRH2 |
| G alpha (s) signalling | transcriptomic | MC5R, ADCYAP1R1, VIP, PTH1R, RAMP3, PTH, |
| events | | GNGT2, ADORA2A, AVP, HTR4, CALCRL, |
| | | RXFP1, SCT, PDE8B, ADRB1, NPS, PDE1B, |
| | | AVPR2, MC2R, VIPR1, RAMP2, ADCYAP1, |
| | | POMC, GIPR, RXFP2, GNG7, SCTR, GNAI2, |
| | | P2RY11, PDE2A, PDE4A, GNG2, PDE3B, ADRB2, |
| | | PDE4D, PDE7B, PDE7A |
| metabolism of proteins | both | RPL35A, TBCE, EIF2B2, EIF4E, PIGT, EIF4EBP1, |
| | | EIF5A, EIF3D, PIGU, RPS15A, DHPS, CCT5, |
| | | RPL10A, AP3M1, RPL19, EIF3E, PIGK, TCP1, |
| | | CCT8, TUBA1C, EIF2S1, RPL8, ARFGEF2, |
| | | TUBA1B, PIGG, RPL21, RPS29, RPL17, RPL26L1, |
| | | RPS27A, TBCA, PIGL, RPS6, RPS5, TUBB3, PIGP, |
| | | PIGS, FBXO4, PIGV, CCT7, RPLP0, PFDN6, |
| | | RPS7, RPS10, NOP56, TBCC, PIGM, TUBB2A, |
| | | EIF4A2, EIF3J, RPL5, PFDN4, CCT2, RPL38, |
| | | PIGB, RPS21, EIF2B5, RPS27, EIF3H, RPS26, |
| | | CCT4 |
| metabolism of proteins | epigenomic | RPL12, PGAP1, EEF2, RPL37, ETF1, EIF1AX, |
| | | DOHH, RPL37A, RPS24, RPS19, RPL32, EIF5B, |
| | | RPL22, RPS13, PLAUR, EIF3A, EEF1A1, FBXL3, |
| | | EIF3B, RPL26, FBXW9, RPL34, PFDN5, RPL29, |
| | | EIF3F, PIGQ, RPLP2, RPS11, DPM3, SPHK1, |
| | | FBXO6, RPS16, RPL9, GGCX, EIF3K, RPL27A, |
| | | RPL11, RPS4X, RPL6, RPLP1, EIF4A1, RPS2, |
| | | UBA52, EIF3G, RPL7, RPL28, PROS1, ACTB, |
| | | FBXW7, EIF4B, RPS23, GPAA1, PFDN1 |
| metabolism of proteins | transcriptomic | RPS4Y1, EEF1G, RPS8, FAU, RPL10, FBXL5, |
| | | PIGW, RPS12, RPSA, RPL31, RPL18A, EIF3I, |
| | | EIF3C, RPL41, RPL24, PIGX, TUBB2B, FBXW10, |
| | | TBCB, RPL30, RPL23, SKIV2L, EIF2B4, RPL4, |
| | | RPS18, RPL36A, RPS3, RPL23A, F2, EIF2B1, |
| | | CCT3, RPS3A, CCT6A, PIGO, LONP2, PIGH, |
| | | VBP1, PIGC, EIF2S2, PIGN, EIF5, EIF2B3, DPM1, |
| | | PIGF, RPL27, EEF1B2, EIF5A2 |
| mRNA splicing | both | HNRNPF, RBM8A, POLR2K, PRPF4, SF3B2, |
| | | POLR2B, MAGOH, TXNL4A, SNRPE, SF3B14, |
| | | SNRPG, YBX1, SF3A3, DDX23, SNRPA1, SNRPB, |
| | | POLR2G, POLR2H, HNRNPR, EFTUD2, NUDT21, |
| | | SNRNP40, U2AF2, POLR2F, CSTF3, NHP2L1, |
| | | POLR2C |
| mRNA splicing | epigenomic | HNRNPA1, SF4, SFRS9, RBM5, HNRNPH1, |
| | | SFRS1, HNRNPM, HNRNPD, SFRS4, UPF3B, |
| | | RNPS1, SNRPF, HNRNPA0, GTF2F1, CCAR1, |
| | | U2AF1, PRPF6, POLR2E, SFRS11, SNRNP70, |
| | | CDC40, HNRNPA3, CPSF1, SFRS6, PCBP2, |
| | | SRRM1, HNRNPA2B1, SFRS3, PCBP1, SF3B1, |
| | | CD2BP2, PTBP1, NCBP2, HNRNPK, PRPF8, |
| | | SNRNP200, FUS, POLR2I |
| mRNA splicing | transcriptomic | CLP1, DNAJC8, DHX9, GTF2F2, SF3B3, SNRPD2, |
| | | SNRPD1, RBMX, SNRPD3, SF3B5, HNRNPC, |
| | | POLR2J, CSTF2, HNRNPL, SNRPB2, CPSF2, |
| | | NCBP1, PHF5A, POLR2D, CSTF1, CPSF3, |

FIGS. 4A-4D show that candidate molecular pathways stratify patients based on response to carboplatin-taxane in an independent cohort. FIG. 4A shows a validation strategy. From left to right, (left) molecular epigenomic profiling of patients, (middle) predicting patients' risk of developing chemoresistance, and (right) informed clinical decision making based on patients personalized risks. FIG. 4B shows t-SNE clustering of lung adenocarcinoma patients treated with carboplatin-taxane (e.g., paclitaxel) from the Tang et al. (Tang et al., Clinical cancer research 2013; 19(6):1577-86) validation cohort (n=39), based on activity levels of 7 candidate pathways. Among the two groups, the green group corresponds to patients with low composite activity levels of candidate pathways, and the orange group corresponds to patients with high composite activity levels of candidate pathways. FIG. 4C shows a Kaplan-Meier survival analysis used to estimate differences in response to carboplatin-taxane (e.g., paclitaxel) between the two patient groups in identified in (FIG. 4B). A log-rank p-value and the number of patients in each group are indicated. FIG. 4D shows two example random models that indicate the non-random predictive ability of the model in the Tang et al. validation cohort: random model 1 (steel-blue) is defined based on to 7 pathways selected at random, and random model 2 (goldenrod) is defined based on to equally—sized patient groups selected at random.

FIGS. 5A-5D show an example comparative performance analysis that confirms the significant predictive ability of pathCHEMO. FIGS. 5A-5B show a comparison of pathCHEMO (turquoise) to other commonly utilized methods, including Panja et al. (Panja et al., EBioMedicine. 2018) Epi2GenR (yellow), Zhong et al. (Zhong et al., Scientific reports. 2018; 8(1):12675) SVM (light blue), Yu et al. (Yu et al., Scientific reports. 2017; 7:43294) PRES random forest (dark blue) using (FIG. 5A) ROC analysis (with AUROC indicated) and (FIG. 5B) Kaplan-Meier and Cox proportional hazards model (with log-rank p-value and hazard ratio indicated) in Tang et al. validation cohort. FIG. 5C shows an example multivariable Cox proportional hazards analysis demonstrating adjustment of 7 candidate pathways for common covariates (i.e., age, gender and stage at diagnosis). The hazard p-value is indicated. FIG. 5D shows a multivariable Cox proportional hazards analysis, demonstrating an adjustment of 7 candidate pathways for signatures of lung cancer aggressiveness, including Larsen et al. (Larsen et al., Clin. Can. Res. 2007; 13(10):2946-54) (54 lung adenocarcinoma markers), Beer et al. (Beer et al., Nature medicine. 2002; 8(8):816-24), (50 lung adenocarcinoma markers), and Tang et al. (Tang et al., Clin. Can. Res. 2013; 19(6):1577-86), (12 non-small cell lung cancer markers). The hazard p-value is indicated.

FIGS. 6A-6C show that pathCHEMO accurately identifies pathways of treatment resistance across chemo-regimens and cancer types. Example treatment-related Kaplan-Meier survival analyses are shown as (FIG. 6A) cisplatin-vinorelbine-treated lung adenocarcinoma (LUAD) patients in the Zhu et al. (Zhu et al., J. Clin. Oncol. 2010; 28(29):4417-24) patient cohort (n=39), (FIG. 6B) cisplatin-vinorelbine-treated lung squamous cell carcinoma (LUSC) patients in the Zhu et al. (Zhu et al., J. Clin. Oncol. 2010; 28(29):4417-24) patient cohort (n=26), and (FIG. 6C) FOLFOX (folinic acid, fluorouracil, and oxaliplatin)-treated colorectal adenocarcinoma (COAD) patients in the Marisa et al. patient cohort (n=23), demonstrating the ability of the identified candidate pathways (for each analysis) to predict treatment response. The log rank p-value and number of patients in each group are indicated.

FIG. 7 shows an example schematic flow representation of pathCHEMO.

FIGS. 8A-8C show example epigenomic alterations in selected candidate molecular pathways of carboplatin-paclitaxel resistance.

FIG. 9 shows a region-based analysis of differentially methylated sites in 7 candidate pathways.

FIGS. 10A-10B show candidate molecular pathways for predicting a response to carboplatin-taxane but that are not predictive of lung cancer aggressiveness.

FIGS. 11A-11G show an example stratified Kaplan-Meier survival analysis, demonstrating independence of the candidate pathways from the common prognostic variables.

FIGS. 12A-12C show identification of pathways of treatment resistance across chemo-regimens and cancer types.

FIG. 13 shows networks of proteins and pathways effected by lung adenocarcinoma cancer and carboplatin-taxane chemotherapy. Larger circles indicate that the protein is more effected by the cancer and chemotherapy.

FIGS. 14A-14C show (top row) example box and whisker plots depicting p-value cutoff for a query chemotherapy response composite methylation pathway signature (x-axis) and NESs from the corresponding GSEA comparison between composite methylation and expression pathways signatures (y-axis). The arrow indicates an optimal p-value threshold with the most significant GSEA enrichment. The bottom row shows example GSEAs comparing a chemotherapy response composite expression pathway signature (reference) and methylation pathway signature. The horizontal red bar in the top left corner indicates leading edge pathways that are altered on both genomic and epigenomic levels. The FIG. 14A data are for LUAD patients with cisplatin-vinorelbine chemotherapy. The genes for each pathway are shown below

Cis + Vin LUAD

| Pathway | source | genes |
|---|---|---|
| actin Y | both | ABI2, ARPC4 |
| actin Y | epigenomic | ACTA1, PSMA7, RAC1, WASF1 |
| actin Y | transcriptomic | ACTR3, ARPC1A, ARPC2, |
| | | ARPC3, WASL |
| metabolism | both | UPP1, GUK1, ADSL, TXNRD1, |
| of nucleotides | | AK5, SLC29A1, TXN, UMPS, |
| | | HPRT1, UCK1, ADSS, TK1 |
| metabolism | epigenomic | DGUOK, APRT, TK2, ADA, |
| of nucleotides | | PFAS, NME4, XDH, |
| | | DPYD, GMPR, CTPS, AMPD2 |
| metabolism | transcriptomic | CMPK1, TYMS, ADK, GART, |
| of nucleotides | | DCK, DTYMK, NME2, IMPDH1, |
| | | PPAT, AK2, GMPS, ATIC, |
| | | SLC29A2, DUT |
| ribosome | both | RPLP2, RPS10, RPS18, RPS15, |
| | | RPL31, RPL24, RPL14, RPS6, |
| | | RPL6, RPS17, RPL41, RPL36, |
| | | RPS25, RPL22L1, RPL36A, |
| | | RPS26, RPL11 |
| ribosome | epigenomic | RPL17 |
| ribosome | transcriptomic | RPL35, RPL38, RPS27, RPS24, |
| | | RPL35A, RPL12, RPL7A, RPL27, |
| | | RPL32, RPL37A, RPS8, RPL39, |
| | | FAU, RPS11, RPL18, RPL8, |
| | | RPL37, RPS27A, RPS19, RPL29, |
| | | RPL26, RSL24D1, RPS21, RPS7, |
| | | RPL23A, RPS13, RPLP0, RPL5, |
| | | RPL15, RPS3A, RPL27A, MRPL13, |
| | | RPL10A, RPL22, RPS3, RPL23, |
| | | RPL13A, RPSA, UBA52, RPL30, |
| | | RPL34, RPL10, RPL21, RPS4X, |
| | | RPS16, RPS29, RPS15A, RPL28, |
| | | RPLP1, RPS23, RPL3, RPS5, |
| | | RPL18A, RPS9, RPS20, RPS12 |

FIG. 14B data are for LUSC patients with cisplatin-vinorelbine chemotherapy. The genes for each pathway are shown below

Cis + Vin LUSC

| Pathway | source | genes |
|---|---|---|
| cytokine- | both | PF4, CCL20, CCL11, CSF3, IL4R, |
| cytokine | | IL19, IL1RAP, LIF, TNFRSF10A, |
| receptor | | CXCL1, CLCF1, IL20, VEGFC, |
| interaction | | IL20RB, PRL, TNFSF18, LEPR, CSF2, |
| | | IL8, LTBR, CXCL5 |
| cytokine- | epi- | GHR, CXCR6, EDA, BMPR1A, |
| cytokine | genomic | VEGFB, CCL21, CCL27, ACVR2A, |
| receptor | | EPOR, CSF1R, IL25, CNTFR, |
| interaction | | TNFSF8, XCL1, CSF2RB, INHBB, PDGFRB, |
| | | IFNA4, INHBC, IL5RA, IL9, CCL13, |
| | | IL2RA, IL18RAP, CCL8, CCR6, CCR1, XCR1, |
| | | IL23R, CCL23, IL3, INHBE, IL26, FIGF, |
| | | XCL2, IL11RA, IL13RA1, CCR8, |
| | | IL17A, ACVRL1, CD70, CCR7, |
| | | CXCR5, IL4, PRLR, HGF, CCR3, IL22, |
| | | CCL14, IL28RA, CXCL13, IFNA14, |
| | | CCL18, CCL7, CCL28, IL12B, CCL4, |
| | | CXCL14, IL21, INHBA, TNFRSF9, IL18, |
| | | LIFR, TNFSF13B, TNFRSF13B, FASLG, |
| | | CSF3R, CCL3, CD40LG, CXCL6, IFNG, |
| | | CCL5, CCL1, TGFBR2, TNFRSF11A, |
| | | TNFSF9, PDGFRA, IL5, KDR, |
| | | IL2RG, TNFRSF10C, CCL19, IL7R, |
| | | GH2, PDGFA, IFNA8, OSM, |
| | | CCL25, IFNA7, IL11, TNFSF11, |
| | | IL28B, CD27, CXCL9, CCL22, IL28A, |
| | | IL10, IL18R1, TGFB2, |
| | | TNFRSF17, CXCL3, CCL2, |
| | | IL21R, CCR9, CCL15, TNFRSF25, ILIB, |
| | | PLEKHO2 |
| cytokine- | trans- | MET, IL1R2, IFNE, RELT, EGFR, |
| cytokine | criptomic | TNFRSF10D, IFNGR1, |
| receptor | | IFNA13, IL1A, TNFRSF12A, TNFRSF1A, |
| interaction | | CCL3L1, FAS, TNFRSF10B, IFNAR1, IFNB1, |
| | | CCL3L3, CCL16, IL6ST, IL10RB, EDAR, |
| | | IL6, CCL24, TNFRSF6B, TNFRSF21, |
| | | BMPR1B, NGFR, |
| | | IL6R, BMPR2, IL1R1, IL24, CXCL11, IFNA2, |
| | | TNFRSF14, PPBP, CCL26 |
| neuroactive | both | GRIK3, OPRK1, CHRNA6, GRID2, OPRL1, |
| ligand- | | GABBR2, AVPR1B, CNR1, NPY5R, GRIA2, |
| receptor | | TACR1, CCKBR, CHRM5, HRH3, CRHR1, |
| interaction | | CCKAR, CNR2, GRIK2, GABRG3, NMUR2, |
| | | PRLR, GABBR1, GLP1R, CHRNA3, GRIK4, |
| | | CHRNB2, GPR156, DRD3, GRIK5, GALR3, |
| | | CHRNA1, CHRNA5, P2RY4, GRM8, |
| | | GABRR1, ADRA1D |
| neuroactive | epi- | GHR, LEPR, PRL, F2, GABRA3, |
| ligand- | genomic | AGTR2, FSHB, CYSLTR2, |
| receptor | | CYSLTR1, PLG, LHCGR, GABRA1, |
| interaction | | SSTR5, HRH2, CHRND, |
| | | SSTR2, HTR1E, CTSG, |
| | | GNRHR, HCRTR1, P2RY6, CHRM4, LPAR3, |
| | | P2RX1, FSHR, GRM4, |
| | | GRPR, GRIN2A, PTGFR, |
| | | CHRNA7, PTGDR, MC5R, APLNR, PTGER4, |
| | | OPRM1, RXFP2, TAAR9, TAAR8, GRIN2C, |
| | | TSHR, NPFFR1, TRHR, ADRB2, |
| | | CHRNG, GRM3, THRB, PTH1R, |
| | | GPR35, CHRNA2, MC4R, SSTR3, |
| | | VIPR2, S1PR2, GABRB2, HTR1D, MCHR2, |
| | | GABRR2, ADRB3, GHSR, CHRNB3, C3AR1, |
| | | GLP2R, ADORA2A, TACR3, P2RY10, |
| | | THRA, LPAR4, PTGIR, ADCYAP1R1, |
| | | GRIA3, CHRNA4, GZMA, |
| | | CGA, HTR4, P2RX3, GRID1, BDKRB2, |
| | | HRH4, TSHB, TAAR5, |
| | | SCTR, GHRHR, GABRG1, |
| | | MTNR1B, P2RY14, CSH1, PRSS1, P2RX6, |
| | | GABRD, HTR6, GRIA4 |
| neuroactive | trans- | GH2, NPY1R, DRD4, DRD5, GRM1, GRIA1, |
| ligand- | criptomic | DRD2, GLRA2, LPAR2, |
| receptor | | GRM5, F2RL2, CHRNE, |
| interaction | | RXFP1, NTSR2, GABRA5, ADRA2B, SSTR1, |
| | | GPR50, SSTR4, GRM7, MTNR1A, NPBWR1, |
| | | VIPR1, CHRNA9, LEP, GABRA4, P2RX5, |
| | | GLRA3, P2RX2, MCHR1, MC3R, CHRNB4, |
| DNA repair | both | POLR2G, FANCA, CCNH, CCNO, POLE2, |
| | | FANCB, MRE11A, FANCL, GTF2H3, |
| | | XRCC5, FANCM, FANCG |
| DNA repair | epi- | MUTYH, FANCI, POLR2I, |
| | genomic | BRCA1, LIG3, REV1, POLDI, |
| | | POLR2H, ALKBH3, LIG1, POLR2C, |
| | | ATM, TP53BP1, XRCC1 |
| DNA repair | trans- | RPA2, POLR2A, REV3L, GTF2H1, |
| | criptomic | ERCC1, POLD3, NBN, POLR2L, |
| | | FANCC, RAD51, MPG, |
| | | MDC1, ERCC8, TCEA1, MNAT1, MAD2L2, |
| | | ALKBH2, RFC3, RAD23B, CDK7, NTHL1, |
| | | XPA, ERCC5, POLD2, USP1, FANCF, |
| | | GTF2H2, PRKDC, RAD52, |
| | | GTF2H4, RFC1, DDB1, POLR2E, MGMT, |
| | | POLR2K, LIG4 |
| SLC- | both | SLC7A9, SLC13A5, SLC4A1, |
| mediated | | SLC1A2, SLC17A8, SLC24A1, SLC6A13, |
| trans- | | SLC4A8, SLC4A5, SLC5A9, SLC24A5, |
| membrane | | SLC4A3, SLC4A7, SLC5A7, SLC44A1, |
| transport | | SLC39A6, SLC6A11, SLC24A3, SLC6A12, |
| | | SLC7A3, SLC30A8, SLC4A9, |
| | | SLC13A2, SLC1A3, |
| | | SLC6A14, SLC30A3, SLC13A4, SLC2A11, |
| | | SLC38A3, SLC43A1, SLC39A8, SLC4A4 |
| SLC- | epi- | SLC12A4, SLC32A1, SLC39A3, SLC44A4, |
| mediated | genomic | SLC13A1, SLC30A2, SLC17A1, SLC1A7, |
| trans- | | SLC7A10, SLC34A2, SLC2A9, SLC34A1, |
| membrane | | SLC16A7, SLC12A1, SLC26A9, SLC8A3, |
| transport | | SLC17A7, SLC26A3, SLC2A4, SLC16A10, |
| | | SLC8A1, SLC9A1, SLC38A4, |
| | | SLC36A2, SLC38A5, |
| | | SLC44A2, SLC2A3, SLC6A20, SLC6A18, |
| | | SLC17A5, SLC1A1, SLC22A2, |
| | | SLC8A2, SLC16A1, SLC6A1, SLC24A4, |
| | | SLC13A3, SLC5A1, SLC10A6 |
| SLC- | trans- | SLC15A1, RHBG, SLC9A9, |
| mediated | criptomic | SLC39A10, SLC6A9, |
| trans- | | SLC2A13, SLC6A3, SLC30A5, SLC12A2, |
| membrane | | SLC34A3, SLC6A15, SLC2A8, SLC2A2, |
| transport | | SLC2A1, SLC2A12, SLC12A5, SLC5A2, |
| | | SLC2A10, SLC6A6, |
| | | SLC26A6, SLC3A1, SLC1A6, |
| | | SLC11A2, SLC44A5, |
| | | SLC16A8, SLC9A4, SLC4A10, SLC14A2, |
| | | SLC30A6 |
| translation | both | RPLP2, RPL31, RPS9, RPS20, |
| | | EIF2B2, RPL30, RPL39, EEF1A1, |
| | | RPL27A, RPL10, FAU, EIF4E, |
| | | RPS28, RPL5, PABPC1, RPS13, |
| | | RPL34, RPL36, EIF5, RPL22 |
| translation | epi- | EIF4A2, RPS21, EIF2B5, RPS3A, RPS25, |
| | genomic | RPSA, RPS26, RPL41, RPS16, RPS15A, |
| | | RPS4Y1, EIF2S3, |
| | | EIF4EBP1, RPL14, EIF2S2, RPL24, RPL23A, |
| | | RPL21, RPL35A, RPL36A, RPLP1, RPS19, |
| | | EIF4G1, EIF1AX, RPL38, RPS17 |
| translation | trans- | EIF2B3, RPS12, EEFIG, RPL7A, |
| | criptomic | RPL11, RPS2, RPS3, |
| | | EIF3I, EEF1B2, RPS8, EIF2S1, RPL18, |
| | | RPS29, RPS10, EIF3J, RPL13, RPL12, |
| | | RPL37A, EIF3E, EIF3H, RPLP0, |
| | | RPL26, RPS27A, RPS18, |
| | | RPL27, EIF3A, EEFID, RPL3, RPS7, RPL19, |
| | | RPS4X, RPL7, RPL10A, RPL6, RPS24, RPL35, |
| | | RPL8, EIF4A1, RPL28, RPL13A, |
| | | RPS11, RPL18A, |
| | | EIF2B1, RPS6, RPS27, RPS15, EIF3C, RPS5, |
| | | RPS14, RPL32, RPL4, EEF2 |
| transport of | both | SEH1L, NCBP1, EIF4E, SRRM1, |
| mature | | NUP54, NUP37, |
| mRNA | | MAGOH, NUP43 |
| derived | | |
| from | | |
| an intron- | | |
| containing | | |
| transcript | | |
| transport of | epi- | SFRS11, RANBP2, SFRS4, NUP133, NUP93, |
| mature | genomic | U2AF2, NCBP2, NUP188, NUP205, NUP155, |
| mRNA | | NUP62, NUPL1, NUP35, |
| derived | | NUPL2, NUP88, SFRS3, |
| from | | UPF3B, THOC4, RAE1, POM121, NUP160, |
| an intron- | | NUP210, SFRS2, NUP153 |
| containing | | |
| transcript | | |
| transport of | trans- | RNPS1, NFX1, AAAS, U2AF1 |
| mature | criptomic | |
| mRNA | | |
| derived | | |
| from | | |
| an intron- | | |
| containing | | |
| transcript | | |

FIG. 14C data are for COAD patients with FOLFOX chemotherapy. The genes for each pathway are shown below

FOLFOX COAD

| Pathway | source | Genes |
|---|---|---|
| elongation | both | SF3B1, POLR2B |
| and | | |
| processing | | |
| of capped | | |
| transcripts | | |
| elongation | epi- | U2AF2, TH1L, SFRS9, CDK7, |
| and | genomic | CCAR1, NHP2L1, U2AF1, SF3A1, |
| processing | | GTF2H4, POLR2C, CD2BP2, |
| of capped | | POLR2G, HNRNPK, SNRNP40, TCEB1, |
| transcripts | | RBM8A, HNRNPH1, SFRS3, PCBP2, |
| | | SNRPA1, SUPT4H1, HNRNPA3, |
| | | SFRS2, HNRNPA1, SNRPB, POLR2K, |
| | | SNRPG, PRPF4, MAGOH, HNRNPD, |
| | | COBRA1, NCBP2, DHX9, NUDT21, |
| | | CCNT2, PCBP1, PRPF8, |
| | | PAPOLA, SUPT16H, HNRNPA2B1, SNRPB2, |
| | | HNRNPR, SRRM1, POLR2F, HNRNPF, |
| | | SF3B2, GTF2F2, CDC40, YBX1, |
| | | SNRPD1, SFRS1, TCEB2 |
| elongation | trans- | ELL, SNRPD2, FUS, POLR2D, SNRNP70, |
| and | criptomic | POLR2J, SF3B4, DDX23, ERCC2, |
| processing | | CPSF1, EFTUD2, GTF2F1, |
| of capped | | SNRPA, PRPF6, RNPS1, SUPT5H, CSTF1, |
| transcripts | | SNRNP200, POLR2I, POLR2H, SF3A2, |
| | | POLR2E, SF3B5, ERCC3, HNRNPM, |
| | | CTDP1, SNRPF, PTBP1, SNRPE, CSTF2, |
| | | CDK9, HNRNPL, HNRNPA0, SF3B3 |
| calcium | both | MYLK3 |
| signaling | | |
| calcium | epi- | P2RX3, SLC25A31, ADCY3, PLCD1, PTAFR, |
| signaling | genomic | LTB4R2, CALML5, ADCY7, |
| | | GNAS, NOS3, DRD5, GRM1, |
| | | HTR2C, TNNC2, P2RX1, TACR3, GRM5, |
| | | P2RX5, CHRM5, CD38, PLCB2 |
| calcium | trans- | ADRA1D, F2R, PTGFR, ITPR2, CACNA1A, |
| signaling | criptomic | PPP3CB, AVPR1A, CAMK2D, ADRA1A, |
| | | P2RX6, P2RX7, PDGFRA, PLN, |
| | | CALM2, CHRNA7, ADRA1B, VDAC2, BST1, |
| | | PDE1A, ADCY8, HTR7, TBXA2R, |
| | | EDNRA, PDE1C, CAMK2A, PDE1B, |
| | | GNAQ, PPP3CA, PTGER3, TRPC1, CHRM2, |
| | | SLC8A1, PPID, CALM1, GRIN1, MYLK, |
| | | DRD1, HTR6, CACNA1E, ADCY2, ATP2B1, |
| | | ATP2B4, ATP2B2, HTR2A, LHCGR, |
| | | AGTR1, HRH2, PPP3R1, |
| | | PDGFRB, ADCY1, ITPR1, NOS1, CAMK2G, |
| | | ATP2B3, ADRB3, PHKB, |
| | | CCKAR, TACR2, ITPKB, RYR2, |
| | | CACNA1H, PRKCA, RYR3, CACNA1B, |
| | | EGFR, PRKCB, RYR1, HTR5A, PPP3R2, |
| | | PRKACG, TRHR, TACR1, PPP3CC, |
| | | SLC25A4, GNAL, GRPR, CAMK4, |
| | | HTR2B, AVPR1B, SLC8A2, CACNA1C, |
| | | NTSR1, GRIN2A, CYSLTR2, EDNRB, |
| | | PLCE1, PLCD4 |
| metabolism | both | RPLP2, TUBB2A |
| of protein | | |
| metabolism | epi- | EIF2S2, RPS4X, EIF3B, UBA52, |
| of protein | genomic | EIF3K, GPAA1, RPL38, EEF2, |
| | | DPM2, PIGF, RPL5, EIF4G1, RPL3, |
| | | FBXO4, EIF2B1, RPS6, TUBB2B, EIF3J, |
| | | TBCE, RPL37, EIF5B, RPL21, |
| | | EIF3A, RPL27, RPL34, RPL29, LONP2, |
| | | EEF1G, PIGP, EIF4A2, RPL26, RPSA, |
| | | TUBA1B, RPL22, RPS27, RPS14, RPL9, |
| | | PIGK, RPS3A, TBCC, ETF1, RPS23, |
| | | RPL31, EIF4H, PGAP1, TUBA1A, |
| | | RPS9, EIF2B2, PFDN5, PIGV, EIF2B3, |
| | | RPL7, PFDN1, EIF5A, RPL26L1, PIGS, |
| | | RPL11, EIF3H, CCT4, EEF1B2, |
| | | EIF3E, EIF4B, PIGB, RPLP0, PIGL, |
| | | SPHK1, RPL14, RPS5, FBXL3, EIF5A2, |
| | | TUBB2C, CCT5, GGCX, EIF1AX, RPL32, |
| | | TBCA, RPL6, PROS1, EIF3F, AP3M1, |
| | | EIF3D, RPL10A |
| metabolism | trans- | EIF3G, PIGG, PIGQ, SKIV2L, TUBA4A, |
| of protein | criptomic | DOHH, RPS28, FBXW9, PFDN2, PIGO, |
| | | PIGC, RPL36, PIGT, DHPS, PIGU, |
| | | RPL23A, RPS21, PROC, EIF2B5, TBCD, |
| | | RPS15, RPS19, TUBA1C, FBXO6, DPM3, |
| | | RPL28, TBCB, FURIN, EIF4EBP1, |
| | | GSPT2, F7, RPS29, RPS26, RPS2, |
| | | PABPC1, TUBB3, CCT3, RPL3L, CCT7, |
| | | RPLP1, RPL19, FBXW5, RPL10, |
| | | PLAUR, EIF3I, RPL37A, ARFGEF2, PIGM |
| processing | both | SF3A1, POLR2B, POLR2E |
| of capped | | |
| intron | | |
| containing | | |
| pre mRNA | | |
| processing | epi- | U2AF2, SNRPB, SNRPG, NUPL2, |
| of capped | genomic | SFRS9, CCAR1, NHP2L1, U2AF1, |
| intron | | POLR2C, CD2BP2, POLR2G, HNRNPK, |
| containing | | SF3B1, SNRNP40, RBM8A, HNRNPH1, |
| pre mRNA | | SFRS3, PCBP2, SNRPA1, HNRNPA3, SFRS2, |
| | | HNRNPA1, POLR2K, PRPF4, |
| | | MAGOH, HNRNPD, |
| | | NCBP2, DHX9, NUDT21, PRPF8, PAPOLA, |
| | | HNRNPA2B1, SNRPB2, HNRNPR, SRRM1, |
| | | POLR2F, HNRNPF, SF3B2, |
| | | GTF2F2, CDC40, YBX1, |
| | | SNRPD1, SFRS1, NUP160, NUP205, NUP107, |
| | | NUP133, RANBP2, NUP155, NUP43, SLBP, |
| | | HNRNPUL1, HNRNPU, SF3B14, NXF1, |
| | | NUP214, NUP153 |
| processing | trans- | SNRPD2, FUS, NUP62, POLR2D, SNRNP70, |
| of capped | criptomic | POLR2J, SF3B4, DDX23, |
| intron | | NUP85, CPSF1, EFTUD2, GTF2F1, |
| containing | | SNRPA, NUP210, PRPF6, RNPS1, CSTF1, |
| pre mRNA | | SNRNP200, POLR2I, POLR2H, SF3A2, RAE1, |
| | | SF3B5, HNRNPM, AAAS, |
| | | SNRPF, PTBP1, POM121, SNRPE, CSTF2, |
| | | NUP188, HNRNPL, HNRNPA0, SF3B3, |
| | | DHX38, CLP1, POLR2L, LSM2, PCBP1 |
| S Phase | both | PSMD1, POLA2, CDC25B |
| S Phase | epi- | MCM4, FEN1, PSMB9, POLD1, |
| | genomic | CDK4, PSMC4, LIG1, CDC6, |
| | | PSMB2, PSMB7, RFC3, GINS2, |
| | | MCM7, PSMC5, CDK7, ORC4L, PSMC2, |
| | | PSME1, CDK2, PSMA6, PSMB6, |
| | | PSMD12, RFC4, PSMC6, PSMB5, |
| | | CCNA2, SKP1, PSMB1, PSMC1, UBB, |
| | | PSMC3, POLD3, ORC6L, |
| | | PRIM1, ORC2L, RFC1, ORC5L, CUL1, |
| | | PSMA4, RPA2, PRIM2, ORC1L, |
| | | PSMD9, CCNE2, PSMD7 |
| S Phase | trans- | FZR1, MCM3, PSME3, PSMD3, PSMB3, |
| | criptomic | PSMD14, PSMB4, CDKN1A, PSMA7, POLE, |
| | | PSMD8, POLD2, GINS4, CCNE1, PSMD11, |
| | | POLD4, PSMD2, PSMD4, MCM2, |
| | | CDC25A, CCND1, CDT1, MCM6, RFC2, |
| | | PSMB10, MCM5, CKS1B, PSMB8 |

### SEQUENCE LISTING

The nucleic sequences listed in the accompanying sequence listing are shown using standard letter abbreviations for nucleotide bases, as defined in 37 C.F.R. 1.822. Only one strand of each nucleic acid sequence is shown, but the complementary strand is understood as included by any reference to the displayed strand. The sequence listing provided herewith, generated on May 27, 2020, 57.7 kb, is herein incorporated by reference.

SEQ ID NO: 1 is an exemplary CCL22 coding sequence.

SEQ ID NO: 2 is an exemplary CCR9 coding sequence.

SEQ ID NO: 3 is an exemplary POLR2C coding sequence.

SEQ ID NO: 4 is an exemplary FGFR1OP coding sequence.

SEQ ID NO: 5 is an exemplary PDE7A coding sequence.

SEQ ID NO: 6 is an exemplary DTYMK coding sequence.

SEQ ID NO: 7 is an exemplary ARPC1A coding sequence.

SEQ ID NO: 8 is an exemplary RPLP2 coding sequence.

SEQ ID NO: 9 is an exemplary ERCC1 coding sequence.

SEQ ID NO: 10 is an exemplary U2AF1 coding sequence.

SEQ ID NO: 11 is an exemplary SF3B3 coding sequence.

SEQ ID NO: 12 is an exemplary PRPF6 coding sequence.

SEQ ID NO: 13 is an exemplary CDC25B coding sequence.

SEQ ID NO: 14 is an exemplary MYLK3 coding sequence.

SEQ ID NO: 15 is an exemplary LSM7 coding sequence.

SEQ ID NO: 16 is an exemplary GABRA1 coding sequence.

SEQ ID NO: 17 is an exemplary SLC44A4 coding sequence.

SEQ ID NO: 18 is an exemplary RPL14 coding sequence.

SEQ ID NO: 19 is an exemplary PFDN1 coding sequence.

SEQ ID NO: 20 is an exemplary CCT4 coding sequence.

SEQ ID NO: 21 is an exemplary CCL11 coding sequence.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. referred to herein are the sequences available at least as early as Jun. 21, 2019. All references, including journal articles, patents, and patent publications, and GenBank® Accession numbers cited herein are incorporated by reference in their entirety.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided.

Actin-related protein 2/3 complex subunit 1A (ARPC1A): Also known as SOP2-LIKE (SOP2L), Epididymis Secretory Sperm Binding Protein 3, Epididymis Secretory Protein Li 307 3 (HEL-S-307 3), Epididymis Luminal Protein 68 3 (HEL-68 3), and Arc40 3 (for example, OMIM no. 604220), ARPC1A aids in regulating actin polymerization in cells and is involved in the actin Y pathway. ARPC1A nucleic acids and proteins are included. Exemplary ARPC1A DNA, mRNA, and proteins include GENBANK® sequences AY407874.1, NM 006409.4, and Q92747.2, respectively. Other ARPC1A molecules are possible. One of ordinary skill in the art can identify additional ARPC1A nucleic acid and protein sequences, including ARPC1A variant that retain biological activity (such as involvement in the actin Y pathway). In some examples, ARPC1A is upregulated (e.g., ARPC1A mRNA expression is increased) in a lung adenocarcinoma that will respond to cisplatin and vinorelbine combination chemotherapy, as compared to ARPC1A mRNA expression in a lung adenocarcinoma that will not respond to cisplatin and vinorelbine combination chemotherapy.

Administration/delivery: To provide or give a subject an agent or therapy by any effective route. Examples of agents include chemotherapy, surgery, radiation therapy, targeted therapy, immunotherapy, or palliative care. Administration includes acute and chronic administration as well as local and systemic administration. In some examples, administration of a therapeutic agent, such as chemotherapy, is by injection (e.g., intravenous, intramuscular, intraosseous, intratumoral, or intraperitoneal). In some examples, administration therapeutic agent, such as chemotherapy, is oral, transdermal, or rectal.

Adenocarcinoma: Carcinoma derived from glandular tissue or in which the tumor cells form recognizable glandular structures. Adenocarcinomas can be classified, according to the predominant pattern of cell arrangement as papillary, alveolar, etc., or according to a particular product of the cells, as mucinous adenocarcinoma. Adenocarcinomas arise in several tissues, including the kidney, breast, colon, cervix, esophagus, gastric, pancreas, prostate, and lung.

Animal: Living multi-cellular vertebrate organisms, a category that includes, for example, mammals and birds. The term mammal includes both human and non-human mammals. Similarly, the term “subject” includes both human and veterinary subjects.

C-C motif chemokine receptor 9 (CCR9): Also known as C-C chemokine receptor type 9 (CC-CKR-9), cluster of differentiation w199 (CDw199), G protein-coupled receptor 9-6 (GPR-9-6), and G protein-coupled receptor 28 (GPR28; for example, OMIM no. 604738), CCR9 is a member of the beta chemokine receptor family and is involved in the immune network for IgA production and chemokine receptor pathways. CCR9 nucleic acids and proteins are included. Exemplary CCR9 DNA, mRNA, and proteins include GENBANK® sequences AY242127.1, NM 031200.3, and AA092294.1, respectively. Other CCR9 molecules are possible. One of ordinary skill in the art can identify additional CCR9 nucleic acid and protein sequences, including CCR9 variant that retain biological activity (such as involvement in the immune network for IgA production and chemokine receptor pathways). In some examples, CCR9 is downregulated (e.g., expression of CCR9 mRNA is decreased) and methylation is increased (e.g., increased CCR9 DNA methylation) in a lung adenocarcinoma that will respond to carboplatin and paclitaxel combination chemotherapy, as compared to such expression and methylation in a lung adenocarcinoma that will not respond to carboplatin and paclitaxel combination chemotherapy.

C-C motif chemokine 11 (CCL11): Also known as small inducible cytokine subfamily A member 11 (SCYA11), eotaxin, and eosinophil chemotactic protein (for example, OMIM no. 601156, CCL11 recruits eosinophils and is involved in the cytokine-cytokine receptor interaction pathway. CCL11 nucleic acids and proteins are included. Exemplary CCL11 DNA, mRNA, and proteins include GENBANK® sequences EF064768.1, NM_002986.3, and CAG33702.1, respectively. Other CCL11 molecules are possible. One of ordinary skill in the art can identify additional CCL11 nucleic acid and protein sequences, including CCL11 variant that retain biological activity (such as involvement in the cytokine-cytokine receptor interaction pathway). In some examples, CCL11 is downregulated (e.g., CCL11 mRNA expression is decreased) and methylation is increased (e.g., increased CCL11 DNA methylation) in lung squamous cell carcinoma that will respond to cisplatin and vinorelbine combination chemotherapy as compared to such expression and methylation in a lung squamous cell carcinoma that will not respond to cisplatin and vinorelbine combination chemotherapy.

C-C motif chemokine ligand 22 (CCL22): Also known as small inducible cytokine subfamily a member 22 (SCYA22) and macrophage-derived chemokine (MDC; for example, OMIM no. 602957), CCL22 is secreted by dendritic cells and macrophages and is involved in the chemokine receptor pathway. CCL22 nucleic acids and proteins are included. Exemplary CCL22 DNA, mRNA, and proteins include GENBANK® sequences EF064764.1, NM_002990.5, and EAW82918.1, respectively. Other CCL22 molecules are possible. One of ordinary skill in the art can identify additional CCL22 nucleic acid and protein sequences, including CCL22 variant that retain biological activity (such as involvement in the chemokine receptor pathway). In some examples, CCL22 is downregulated (e.g., CCL22 mRNA expression is decreased) and methylation is increased (e.g., increased CCL22 DNA methylation) in a lung adenocarcinoma that will respond to carboplatin and paclitaxel combination chemotherapy, as compared to such expression and methylation in a lung adenocarcinoma that will not respond to carboplatin and paclitaxel combination chemotherapy.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system. In one example, cancer cells, for example lung or colorectal cancer cells, are analyzed by the disclosed methods.

Cell division cycle 25B (CDC25B): CDC25B is a phosphatase that activates CDK1-cyclin B and is involved in the S phase pathway (for example, OMIM no. 116949). CDC25B nucleic acids and proteins are included. Exemplary CDC25B DNA, mRNA, and proteins include GENBANK® sequences AY494082.1, M81934.1, and P30305.2, respectively. Other CDC25B molecules are possible. One of ordinary skill in the art can identify additional human, mouse, and rat CDC25B nucleic acid and protein sequences, including CDC25B variant that retain biological activity (such as involvement in the S phase pathway). In some examples, CDC25B is upregulated (e.g., CDC25B mRNA expression is increased) and CDC25B methylation is decreased (e.g., decreased CDC25B DNA methylation) in a colon adenocarcinoma that will respond to FOLFOX combination chemotherapy, as compared to such expression and methylation in a colon adenocarcinoma that will not respond to FOLFOX combination chemotherapy.

Chaperonin-containing TCP1 subunit 4 (CCT4): Also known as CCT-delta (CCTD) and stimulator of TAR RNA-binding proteins (SRB; for example, OMIM no. 605142), CCT4 aids in protein folding as is involved in the protein metabolism pathway. CCT4 nucleic acids and proteins are included. Exemplary CCT4 DNA, mRNA, and proteins include GENBANK® sequences AC107081.5, NM_006430.4, and P50991.4, respectively. Other CCT4 molecules are possible. One of ordinary skill in the art can identify additional CCT4 nucleic acid and protein sequences, including CCT4 variant that retain biological activity (such as involvement in the protein metabolism pathway). In some examples, CCT4 is upregulated (e.g., CCT4 mRNA expression is increased) and CCT4 methylation is decreased (e.g., decreased CCT4 DNA methylation) in a lung adenocarcinoma carcinoma that will respond to carboplatin and paclitaxel combination chemotherapy, as compared to CCT4 expression and methylation in a lung adenocarcinoma that will not respond to carboplatin and paclitaxel combination chemotherapy.

Chemotherapeutic agent or Chemotherapy: Any chemical or biological agent with therapeutic usefulness in the treatment of diseases characterized by abnormal cell growth. Such diseases include tumors, neoplasms, and cancer. In one embodiment, a chemotherapeutic agent is an agent of use in treating lung cancer, such as lung adenocarcinoma or lung squamous cell carcinoma. In one embodiment, a chemotherapeutic agent is an agent of use in treating colorectal cancer, such as colorectal adenocarcinoma. In some examples, chemotherapeutic agents include carboplatin, paclitaxel, cisplatin, vinorelbine, folinic acid, fluorouracil, or oxaliplatin, in any combination together or with other agents. In some examples, the chemotherapeutic agents include a combination of carboplatin and paclitaxel, a combination of cisplatin and vinorelbine, and a combination of folinic acid, fluorouracil, and oxaliplatin. Exemplary chemotherapeutic agents are provided in Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993, all incorporated herein by reference. Combination chemotherapy is the administration of more than one agent (such as more than one chemical chemotherapeutic agent) to treat cancer. Such a combination can be administered simultaneously, contemporaneously, or with a period of time in between.

Colorectal cancer: Also known as bowel or colon cancer, colorectal cancer includes cancer from the colon, rectum, or parts or the large intestine. Examples of colon cancer include adenocarcinoma, lymphoma, adenosquamous cell carcinoma, and squamous cell carcinoma. A variety of therapies can be used to treat colorectal cancer, including surgery, chemotherapy (such as folinic acid, fluorouracil, and oxaliplatin, for example, to treat colon adenocarcinoma), radiation therapy, targeted drug therapy, immunotherapy, and palliative care.

Control: A reference standard. In some embodiments, the control is a healthy subject. In other embodiments, the control is a subject with a cancer, such as a lung or colon cancer. In some embodiments, the control is a subject who responds positively to chemotherapy, such as a subject who does not develop resistance to chemotherapy. In other embodiments, the control is a subject who does not respond positively to chemotherapy, such as a subject who develops resistance to chemotherapy. In still other embodiments, the control is a historical control or standard reference value or range of values (e.g., a previously tested control subject with a known prognosis or outcome or group of subjects that represent baseline or normal values). A difference between a test subject and a control can be an increase or a decrease. The difference can be a qualitative difference or a quantitative difference, for example a statistically significant difference.

Deoxythymidylate kinase (DTYMK): Also known as thymidylate kinase (TYMK) and CDC8 (for example OMIM no. 188345), DTYMK catalyzes phosphorylation of dTMP and is involved in the nucleotide metabolism pathway. TYMK nucleic acids and proteins are included. Exemplary TYMK DNA, mRNA, and proteins include GENBANK® sequences DQ052285.1, CR542015.1, and CAG46783.1, respectively. Other TYMK molecules are possible. One of ordinary skill in the art can identify additional TYMK nucleic acid and protein sequences, including TYMK variant that retain biological activity (such as involvement in the nucleotide metabolism pathway). In some examples, DTYMK is upregulated (e.g., DTYMK mRNA expression is increased) in a lung adenocarcinoma that will respond to cisplatin and vinorelbine combination chemotherapy, as compared to DTYMK mRNA expression in a lung adenocarcinoma that will not respond to cisplatin and vinorelbine combination chemotherapy.

Detect: To determine if an agent (such as a signal; particular nucleotide; amino acid; nucleic acid molecule and/or nucleotide modification, such as a methylated nucleotide; mRNA; or protein) is present or absent. In some examples, detection can include further quantification. For example, use of the disclosed methods in particular examples permits detection of nucleic acid expression (e.g., mRNA expression) or nucleic acid modification (such as DNA methylation) in a sample.

Differential Expression: A nucleic acid molecule is differentially expressed when the amount of one or more of its expression products (e.g., transcript, such as mRNA, and/or protein) is higher or lower in one sample (such as a test lung or colorectal cancer sample) as compared to another sample (such as a control lung or colorectal cancer sample). Detecting differential expression can include measuring a change in gene (such as by measuring mRNA) or protein expression.

Differential methylation: A nucleic acid molecule is differentially methylated when the amount of methylated nucleotides in the gene (such as the gene body) or sequences associated with gene transcription (such as promoters, for example, in CpG islands of promoters) is higher or lower in one sample (such as a test lung or colorectal cancer sample) as compared to another sample (such as a control lung or colorectal cancer sample). Detecting differential methylation can include measuring methylation using a bisulfite conversion assay or any other method of detecting DNA methylation (e.g., Levenson et al., Expert Rev Mol Diagn, 10(4): 481-488, 2010, incorporated herein by reference in its entirety).

Excision repair cross-complementation group 1 (ERCC1): Also known as Excision Repair Cross-Complementing Rodent Repair Deficiency Complementation Group 1, COFS4, RAD10, and UV20 (for example, OMIM no. 126380), ERCC1 is involved in the DNA repair pathway. ERCC1 nucleic acids and proteins are included. Exemplary ERCC1 DNA, mRNA, and proteins include GENBANK® sequences AF512555.1, AF001925.1, and P07992.1, respectively. Other ERCC1 molecules are possible. One of ordinary skill in the art can identify additional ERCC1 nucleic acid and protein sequences, including ERCC1 variant that retain biological activity (such as involvement in the DNA repair pathway). In some examples, ERCC1 expression is downregulated (e.g., ERCC1 mRNA expression is decreased) in a lung squamous cell carcinoma that will respond to cisplatin and vinorelbine combination chemotherapy, as compared to ERCC1 expression in a lung squamous cell carcinoma that will not respond to cisplatin and vinorelbine combination chemotherapy.

Expression: Translation of a nucleic acid into a peptide or protein. Peptides or proteins may be expressed and remain intracellular, become a component of the cell surface membrane, or be secreted into the extracellular matrix or medium.

Fibroblast growth factor receptor 1 oncogene partner (FGFR1OP): Also known as FGFR1 oncogene partner (FOP; OMIM no. 605392), FGFR1OP plays a role in cell proliferation and differentiation and is involved in the mitotic cell cycle pathway. FGFR1OP nucleic acids and proteins are included. Exemplary FGFR1OP DNA, mRNA, and proteins include DQ030392.1, BC037785.1, and AAH11902.1, respectively. Other FGFR1OP molecules are possible. One of ordinary skill in the art can identify additional FGFR1OP nucleic acid and protein sequences, including FGFR1OP variant that retain biological activity (such as involvement in the mitotic cell cycle pathway). In some examples, expression of FGFR1OP is upregulated (e.g., FGFR1OP mRNA expression is increased) and methylation decreased (e.g., FGFR1OP DNA methylation is decreased) in a lung adenocarcinoma carcinoma that will respond to carboplatin and paclitaxel combination chemotherapy as compared to FGFR1OP expression and methylation in a lung adenocarcinoma that will not respond to carboplatin and paclitaxel combination chemotherapy.

Gamma-aminobutyric acid receptor alpha-1 (GABRA1): Also known as GABA-A receptor, alpha-1 polypeptide, EIEE19, ECA4, EJM5, and EJM, GABRA1 is an inhibitory neurotransmitter and is involved in the neuroactive ligand-receptor interaction pathway. GABRA1 nucleic acids and proteins are included. Exemplary GABRA1 DNA, mRNA, and proteins include NG_011548.1, NM_000806.5, and AAH30696.1, respectively. Other GABRA1 molecules are possible. One of ordinary skill in the art can identify additional GABRA1 nucleic acid and protein sequences, including GABRA1 variant that retain biological activity (such as involvement in the neuroactive ligand-receptor interaction pathway). In some examples, methylation of GABRA1 is increased (e.g., increased GABRA1 DNA methylation) in a lung squamous cell carcinoma that will respond to cisplatin and vinorelbine combination chemotherapy, as compared to GABRA1 methylation in a lung squamous cell carcinoma that will not respond to cisplatin and vinorelbine combination chemotherapy.

Inhibiting or treating a disease: Inhibiting the full development of a disease or condition, for example, in a subject who is at risk for a disease, such as a subject with cancer, for example, lung or colon cancer. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

LSM7: Also known as YNL147W, LSM7 homolog U6 small nuclear RNA and mRNA degradation associated (for example, OMIM no. 607287), LSM7 forms an oligomer that interacts with RNA to form a protein-RNA complex and is involved in the RNA degradation pathway. LSM7 nucleic acids and proteins are included. Exemplary LSM7 DNA, mRNA, and proteins include AF182293, NM_016199.3, and NP 057283.1, respectively. Other LSM7 molecules are possible. One of ordinary skill in the art can identify additional LSM7 nucleic acid and protein sequences, including LSM7 variant that retain biological activity (such as involvement in the RNA degradation pathway). In some examples, methylation of LSM7 is decreased (e.g., LSM7 DNA methylation is decreased) in a lung adenocarcinoma carcinoma that will respond to carboplatin and paclitaxel combination chemotherapy as compared to LSM7 DNA methylation in a lung adenocarcinoma that will not respond to carboplatin and paclitaxel combination chemotherapy.

Lung cancer: A cancer of the lung tissue. Lung cancer can be small-cell or non-small cell lung cancer. Examples of non-small cell carcinoma include adenocarcinoma, squamous-cell carcinoma, and large-cell carcinoma. A variety of therapies can be administered to treat or inhibit lung cancers, such as chemotherapy (for example, carboplatin, paclitaxel, cisplatin, vinorelbine, or a combination thereof can be used for treatment, such as carboplatin and paclitaxel, for example to treat lung adenocarcinoma, or cisplatin and vinorelbine, for example, to treat lung adenocarcinoma or lung squamous cell carcinoma), surgery, radiation therapy, targeted therapy, immunotherapy, and palliative care.

Methylation: Methylation of DNA can alter the activity of the DNA without changing the sequence. Two bases in DNA can be methylated, cytosine and adenine. Methylation can be used to either express or repress genes; often methylation of CpG islands in promoters are associated with gene repression, while methylation of a gene body is often associated with high levels of gene transcription.

Myosin light chain kinase 3 (MYLK3): Also known as cardiac MLCK, MYLK3 plays a role in regulating cardiovascular function and is involved in the calcium signaling pathway. MYLK3 nucleic acids and proteins are included. Exemplary MYLK3 DNA, mRNA, and proteins include HF584427.1, AJ247087.1, and Q32MK0.3, respectively. Other MYLK3 molecules are possible. One of ordinary skill in the art can identify additional MYLK3 nucleic acid and protein sequences, including MYLK3 variant that retain biological activity (such as involvement in the calcium signaling pathway). In some examples, expression of MYLK3 is downregulated (e.g., MYLK3 mRNA expression is decreased) and methylation of MYLK3 is increased (e.g., MYLK3 DNA methylation is increased) in a subject with colon adenocarcinoma that will respond to FOLFOX combination chemotherapy, as compared to MYLK3 expression and methylation in a colon adenocarcinoma that will not respond to FOLFOX combination chemotherapy.

Phosphodiesterase 7A (PDE7A): Also known as high affinity CAMP-specific 3′,5′-cyclic phosphodiesterase 7A, human complement of yeast PDE1/PDE2 (HCP1; for example, OMIM no. 171885), PDE7A regulates concentrations of cyclic nucleotides and are involved in the G alpha signaling pathway. PDE7A nucleic acids and proteins are included. Exemplary PDE7A DNA, mRNA, and proteins include NG_029614.1, L12052.1, and Q13946.2, respectively. Other PDE7A molecules are possible. One of ordinary skill in the art can identify additional PDE7A nucleic acid and protein sequences, including PDE7A variant that retain biological activity (such as involvement in the G alpha signaling pathway). In some examples, PDE7A is downregulated (e.g., PDE7A mRNA expression is decreased) in a lung adenocarcinoma that will respond to carboplatin and paclitaxel combination chemotherapy, as compared to PDE7A expression in a lung adenocarcinoma that will not respond to carboplatin and paclitaxel combination chemotherapy.

Pre-mRNA processing factor 6 (PRPF6): Also known as PRP6, androgen receptor n-terminal domain-transactivating protein 1, ANT1, TOM, and chromosome 20 open reading frame 14 (C20ORF14; for example, OMIM no. 613979), PRPF6 binds androgen receptor and is involved in the processing of capped intron containing pre-mRNA pathway. PRPF6 nucleic acids and proteins are included. Exemplary PRPF6 DNA, mRNA, and proteins include NG_029719.1, NM_012469.4, and 094906.1, respectively. Other PRPF6 molecules are possible. One of ordinary skill in the art can identify additional PRPF6 nucleic acid and protein sequences, including PRPF6 variants that retain biological activity (such as involvement in the processing of capped intron containing pre-mRNA pathway). In some examples, PRPF6 is upregulated (e.g., PRPF6 mRNA expression is increased) in a colon adenocarcinoma that will respond to FOLFOX combination chemotherapy, as compared to PRPF6 expression in a colon adenocarcinoma that will not respond to FOLFOX combination chemotherapy.

Prefoldin subunit 1 (PFDN1): PFDN1 (for example, OMIM no. 604897) aids in binding and stabilizing newly synthesized polypeptides and is involved in the protein metabolism pathway. PFDN1 nucleic acids and proteins are included. Exemplary PFDN1 DNA, mRNA, and proteins include AY421527.1, NM_002622.5, and NP_002613.2, respectively. Other PFDN1 molecules are possible. One of ordinary skill in the art can identify additional PFDN1 nucleic acid and protein sequences, including PFDN1 variant that retain biological activity (such as involvement in the protein metabolism pathway). In some examples, methylation of PFDN1 is decreased (e.g., PFDN1 DNA methylation is decreased) in a colon adenocarcinoma that will respond to FOLFOX combination chemotherapy, as compared to PFDN1 methylation in a colon adenocarcinoma that will not respond to FOLFOX combination chemotherapy.

Primer: Short nucleic acids, for example DNA oligonucleotides 10 nucleotides or more in length, which are annealed to a complementary target nucleic acid strand (e.g., of a CCL22, CCR9, POLR2C, FGFR1OP, CCT4, PDE7A, DTYMK, ARPC1A, RPLP2, CCL11, ERCC1, U2AF1, SF3B3, PRPF6, CDC25B, LSM7, GABRA1, SLC44A4, RPL14, PFDN1, or MYLK3 nucleic acid molecule, such any of SEQ ID NOS: 1-21 or their complementary strand) by nucleic acid hybridization to form a hybrid between the primer and the target nucleic acid strand, then extended along the target nucleic acid strand by a polymerase enzyme. Therefore, primers can be used to measure nucleic acid expression. In addition, primer pairs can be used for amplification of a nucleic acid sequence, e.g., by the polymerase chain reaction (PCR) or other nucleic-acid amplification methods.

Primers include at least 10 nucleotides complementary to the target nucleic acid molecule. In order to enhance specificity, longer primers may also be employed, such as primers having 15, 20, 30, 40, 50, 60, 70, 80, 90 or 100 consecutive nucleotides of the complementary nucleic acid molecule to be detected. Methods for preparing and using primers are described in, for example, Sambrook et al. (1989) Molecular Cloning: A Laboratory Manual, Cold Spring Harbor, N.Y.; Ausubel et al. (1987) Current Protocols in Molecular Biology, Greene Publ. Assoc. & Wiley-Intersciences.

In some examples, if the nucleic acid to be detected is DNA, the primer is DNA, RNA, or a mixture of both. In some examples, if the nucleic acid to be detected is RNA, the primer is RNA or DNA.

In some examples, primers include a detectable label, such as a fluorophore or enzyme, and are referred to as probes, which can also be used to detect a target nucleic acid molecule provided herein.

Ribosomal protein lateral stalk subunit P2 (RPLP2): Also known as 60S acidic ribosomal protein P2, large ribosomal subunit protein P2, acidic ribosomal phosphoprotein P2, P2, LP2, renal carcinoma antigen NY-REN-44, RPLP2 is a part of the 60S subunit and is involved in the ribosome pathway. RPLP2 nucleic acids and proteins are included. Exemplary RPLP2 DNA, mRNA, and proteins include DQ036650.1, NM_001004.4, and CAG47008.1, respectively. Other RPLP2 molecules are possible. One of ordinary skill in the art can identify additional RPLP2 nucleic acid and protein sequences, including RPLP2 variants that retain biological activity (such as involvement in the ribosome pathway). In some examples, RPLP2 is upregulated (e.g., RPLP2 mRNA expression is increased) and RPLP2 methylation decreased (e.g., methylation of RPLP2 DNA is decreased) in a subject with lung adenocarcinoma that will respond to cisplatin and vinorelbine combination chemotherapy, as compared to RPLP2 expression and methylation in a lung adenocarcinoma that will not respond to cisplatin and vinorelbine combination chemotherapy.

Ribosomal protein L14 (RPL14): Also known as 60S Ribosomal Protein L14, Large Ribosomal Subunit Protein EL14, CAG-ISL 7, CTG-B33, HRL14, and L14, RPL14 is a part of the 60S subunit and is involved in the translation pathway. RPL14 is a subunit for RNA polymerase and is involved in the RNA splicing pathway. RPL14 nucleic acids and proteins are included. Exemplary RPL14 DNA, mRNA, and proteins includes AB061822.1, BC009294.2, and AAH71913.1, respectively. Other RPL14 molecules are possible. One of ordinary skill in the art can identify additional RPL14 nucleic acid and protein sequences, including RPL14 variants that retain biological activity (such as involvement in the RNA splicing pathway). In some examples, methylation of RPL14 is decreased (e.g., methylation of RPL14 DNA is decreased) in a lung squamous cell carcinoma that will respond to cisplatin and vinorelbine combination chemotherapy, as compared to RPL14 methylation in a lung squamous cell carcinoma that will not respond to cisplatin and vinorelbine combination chemotherapy.

RNA polymerase II subunit C (POLR2C): Also known as DNA-directed RNA polymerase II subunit 3 (RPB3), DNA-Directed RNA Polymerase II 33 KDa Polypeptide (RPB33), RPB31, hRPB33, and hsRPB3 (OMIM no. 180663), POLR2C is a subunit for RNA polymerase and is involved in the RNA splicing pathway. POLR2C nucleic acids and proteins are included. Exemplary POLR2C DNA, mRNA, and proteins include DQ032841.1, CR542041.1, and CAG46838.1, respectively. Other POLR2C molecules are possible. One of ordinary skill in the art can identify additional POLR2C nucleic acid and protein sequences, including POLR2C variants that retain biological activity (such as involvement in the RNA splicing pathway). In some examples, POLR2C is upregulated (e.g., POLR2C mRNA expression is increased) and methylation of POLR2C is decreased (e.g., methylation of POLR2C DNA is decreased) in a lung adenocarcinoma that will respond to carboplatin and paclitaxel combination chemotherapy, as compared to such expression and methylation in a lung adenocarcinoma that will not respond to carboplatin and paclitaxel combination chemotherapy.

Sample or biological sample: A sample of biological material obtained from a subject, which can include cells, proteins, and/or nucleic acid molecules (such as DNA and/or RNA, such as mRNA). Biological samples include all clinical samples useful for detection of disease, such as cancer, in subjects. Appropriate samples include any conventional biological samples, including clinical samples obtained from a human or veterinary subject. Exemplary samples include, without limitation, cancer samples (such as from surgery, tissue biopsy, tissue sections, or autopsy), cells, cell lysates, blood smears, cytocentrifuge preparations, cytology smears, bodily fluids (e.g., blood, plasma, serum, stool/feces, saliva, sputum, urine, bronchoalveolar lavage, semen, cerebrospinal fluid (CSF), etc.), or fine-needle aspirates. Samples may be used directly from a subject, or may be processed before analysis (such as concentrated, diluted, purified, such as isolation and/or amplification of nucleic acid molecules in the sample). In a particular example, a sample or biological sample is obtained from a subject having, suspected of having, or at risk of having cancer (such as lung or colorectal cancer). In a specific example, the sample is a lung cancer sample. In another specific example, the sample is a colorectal cancer sample.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biotechnology (NCBI, National Library of Medicine, Building 38A, Room 8N805, Bethesda, Md. 20894) and on the Internet, for use in connection with the sequence analysis programs blastp, blastn, blastx, tblastn, and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs may use SEG filtering (Wootton and Federhen, Meth. Enzymol. 266:554-571, 1996). In addition, a manual alignment can be performed.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 80%, at least 90%, at least 95%, at least 98%, or at least 99% sequence identity to a molecule listed in the sequence listing, such as any one of SEQ ID NOS: 1-21. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Solute carrier family 44 member 4 (SLC44A4): Also known as choline transporter-like protein 4 (CTL4), thiamine pyrophosphate transporter (TPPT), TPP transporter, chromosome 6 open reading frame 29 (C6ORF29), and testicular tissue protein Li 48 (for example, OMIM no. 606107), SLC44A4 aids in supplying choline to cells and is involved in the solute carrier (SLC)-mediated transmembrane transport pathway. SLC44A4 nucleic acids and proteins are included. Exemplary SLC44A4 DNA, mRNA, and proteins include KY500657.2, NM_200413.1, and AQY77128.1, respectively. Other SLC44A4 molecules are possible. One of ordinary skill in the art can identify additional SLC44A4 nucleic acid and protein sequences, including SLC44A4 variants that retain biological activity (such as involvement in the solute carrier (SLC)-mediated transmembrane transport pathway). In some examples, methylation of SLC44A4 is increased (e.g., SLC44A4 DNA methylation is increased) in a lung squamous cell carcinoma that will respond to cisplatin and vinorelbine combination chemotherapy as compared to a lung squamous cell carcinoma that will not respond to cisplatin and vinorelbine combination chemotherapy.

Splicing factor 3b subunit 3 (SF3B3): Also known as spliceosome-associated protein 130 kd (SAP130; for example OMIM no. 605592), SF3B3 is a component of small nuclear ribonucleoprotein and spliceosome complexes and is involved in the elongation and processing of capped transcripts pathway. SF3B3 nucleic acids and proteins are included. Exemplary SF3B3 DNA includes NG_046937.1, BC068974.1, and Q15393.4, respectively. Other SF3B3 molecules are possible. One of ordinary skill in the art can identify additional SF3B3 nucleic acid and protein sequences, including SF3B3 variants that retain biological activity (such as involvement in the elongation and processing of capped transcripts pathway). In some examples, SF3B3 is upregulated (e.g., SF3B3 mRNA expression is increased) in a colon adenocarcinoma that will respond to FOLFOX combination chemotherapy, as compared to SF3B3 expression in a colon adenocarcinoma that will not respond to FOLFOX combination chemotherapy.

Subject: As used herein, the term “subject” refers to a mammal and includes, without limitation, humans, domestic animals (e.g., dogs or cats), farm animals (e.g., cows, horses, or pigs), and laboratory animals (mice, rats, hamsters, guinea pigs, pigs, rabbits, dogs, or monkeys). In one example, the subject treated and/or analyzed with the disclosed methods has cancer, such as lung or colorectal cancer. In some examples, the subject responds positively to chemotherapy, such as a subject who does not develop resistance to chemotherapy.

Therapeutically effective amount: The amount of an active ingredient (such as a chemotherapeutic agent) that is sufficient to effect treatment when administered to a mammal in need of such treatment, such as treatment of a cancer. The therapeutically effective amount will vary depending upon the subject and disease condition being treated, the weight and age of the subject, the severity of the disease condition, the manner of administration and the like, which can readily be determined by a prescribing physician.

Treating, treatment, and therapy: Any success or indicia of success in the attenuation or amelioration of an injury, pathology, or condition, including any objective or subjective parameter such as abatement, remission, diminishing of symptoms or making the condition more tolerable to the patient, slowing in the rate of degeneration or decline, making the final point of degeneration less debilitating, improving a subject's sensorimotor function. The treatment may be assessed by objective or subjective parameters; including the results of a physical examination, neurological examination, or psychiatric evaluations. For example, treatment of a cancer can include decreasing the size, volume, or weight of a cancer, decrease the number, size, volume, or weight of metastases, or combinations thereof.

Tumor, neoplasia, malignancy or cancer: A neoplasm is an abnormal growth of tissue or cells which results from excessive cell division. Neoplastic growth can produce a tumor. The amount of a tumor in an individual is the “tumor burden”, which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer. Exemplary tumors, such as cancers, that can be analyzed and treated with the disclosed methods include carcinomas of the lung (such as squamous cell carcinoma and adenocarcinoma) and colorectal adenocarcinoma.

U2 small nuclear RNA auxiliary factor 1 (U2AF1): Also known as U2 small nuclear ribonucleoprotein auxiliary factor 35-kd subunit (U2AF35), Splicing Factor U2AF 35 kd Subunit, U2AFBP, U2AF35, RNU2AF1, FP793, and RN, U2AF1 plays a role in RNA splicing and is involved in the transport of mature mRNA derived from an intron-containing transcript pathway. U2AF1 nucleic acids and proteins are included. Exemplary U2AF1 DNA, mRNA, and proteins include NG_029455.1, BC005915.1, and Q01081.3, respectively. Other U2AF1 molecules are possible. One of ordinary skill in the art can identify additional U2AF1 nucleic acid and protein sequences, including U2AF1 variants that retain biological activity (such as involvement in the intron-containing transcript pathway). In some examples, U2AF1 is downregulated (e.g., U2AF1 mRNA expression is decreased) in a lung squamous cell carcinoma that will respond to cisplatin and vinorelbine combination chemotherapy, as compared to U2AF1 expression in a lung squamous cell carcinoma that will not respond to cisplatin and vinorelbine combination chemotherapy.

### Overview

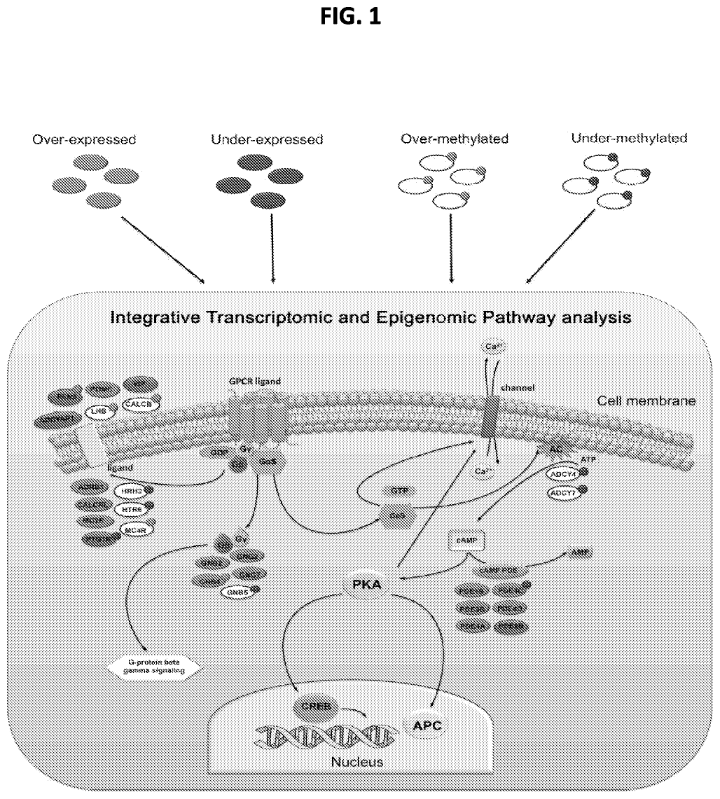

Pathways having altered mRNA expression and DNA methylation levels are more likely to capture complex relationships implicated in therapeutic resistance and overcome noise present in any single experiment or data type (see, for example, FIG. 1). The methods disclosed herein tackle complexity of treatment responses by: (i) identifying molecular pathways altered on both genomic and epigenomic levels, which yields functionally relevant alterations; (ii) the identified pathways are markers of primary chemoresistance for predicting patients with poor and favorable response, even prior to administering therapy; and (iii) molecular pathways, rather than single determinants, are used, which provides functional candidates for therapeutic intervention to preclude or overcome resistance.

Profiles of patients with lung adenocarcinoma (LUAD) from The Cancer Genome Atlas (TCGA-LUAD) (Cancer Genome Atlas Research Network, Nature. 2014; 511(7511):543-50) and treated with the standard-of-care chemotherapy (a combination of platinum-based carboplatin and paclitaxel) were used to yield markers of chemoresponse in lung cancer. In one example, the methods disclosed herein identified seven epigenomically altered pathways that differentiate patients with poor and favorable carboplatin-paclitaxel response (FIG. 13). Shown herein, the activity of these pathways serve as molecular markers for patients at risk of resistance to carboplatin-paclitaxel in an independent patient cohort (Tang et al., Clin. Canc. Res. 2013; 19(6):1577-86) (log-rank p-value=0.0081, hazard ratio=10) with demonstrably high accuracy for predicting the risk of resistance to carboplatin-paclitaxel combination in new patients (for example, using leave-one-out cross-validation). Significant, non-random predictive ability of the identified 7 candidate pathways was confirmed through comparison to 7 pathways selected at random (p-value<0.007). Furthermore, the methods disclosed herein outperform other commonly utilized methods (for example, methods based on linear regression, support vector machine, and random forest) in identifying patients at risk of resistance to chemotherapy (AUROC=0.98) (Panja et al., EBioMedicine. 2018, Yu et al., Scientific reports. 2017; 7:43294, Zhong et al., Scientific reports. 2018; 8(1):12675). In addition, the methods herein are independent of, and are not affected by, common covariates (such as age, gender, and tumor stage at diagnosis) or known signatures of lung cancer aggressiveness (adjusted hazard ratio=14, hazard p-value=0.03). Finally, the methods herein are effective for multiple chemo combinations (for example, a combination of platinum-based cisplatin and plant alkaloid vinorelbine or a combination of platinum-based oxaliplatin and antimetabolite agent fluorouracil) and multiple cancer types (for example, lung squamous cell carcinoma and colorectal adenocarcinoma), which demonstrates the general applicability of the methods disclosed herein (log-rank p-value<0.03, hazard ratio>3.5). Thus, the methods herein can be used to pre-screen patients and prioritize them for specific chemotherapy regimens.

To evaluate clinical effects of these pathways, canSAR (Tym et al., Nucleic Acids Res. 2016; 44(D1):D938-43) a computational chemogenomic analysis, which connects molecular alterations to potential therapeutic targeting with approved or investigational drugs (or drugs considered as candidates for future clinical trials) was used for therapeutic targeting of pathway genes.

Evaluating Expression and Methylation in a Subject with Cancer

Provided herein are methods of identifying a subject with cancer who will respond to chemotherapy (such as a human or veterinary subject). In particular examples, the methods can determine with high accuracy whether a subject has a cancer that is likely to respond to chemotherapy. For example, the methods herein can distinguish between a chemotherapy response with an area under the curve receiver operating characteristics (AUROC) curve of at least 0.85, at least 0.90, at least 0.95, at least 0.96, at least 0.97, at least 0.98, or at least 0.99, such as about 0.95 to about 0.99, with a p value of less than 0.05 or less than 0.01, for LUAD with carboplatin and paclitaxel chemotherapy, for LUAD with cisplatin and vinorelbine chemotherapy, LUSC with cisplatin and vinorelbine chemotherapy or COAD with FOLFOX chemotherapy. In one example, the methods herein distinguish between a chemotherapy response with an AUROC curve of at least or about 0.95 with a p value of less than or about 0.008 for LUAD with carboplatin and paclitaxel chemotherapy. In one example, the methods herein distinguish between a chemotherapy response with an AUROC curve of at least or about 0.97 with a p value of less than or about 0.005 for LUAD with cisplatin and vinorelbine chemotherapy. In one example, the methods herein distinguish between a chemotherapy response with an AUROC curve of at least or about 0.98 with a p value of less than or about 0.026 for LUSC with cisplatin and vinorelbine chemotherapy. In one example, the methods herein distinguish between a chemotherapy response with an AUROC curve of at least or about 0.98 with a p value of less than or about 0.011 for COAD with FOLFOX chemotherapy. The methods herein can be used to treat a variety of cancers with chemotherapy or identify cancers that will respond to chemotherapy. It is helpful to determine whether or not a cancer in a subject is responsive to chemotherapy because there are a variety of protocols for treating cancer but not all are effective for a particular subject's cancer. Hence, using the results of the disclosed methods allows subjects to be administered a therapy or treatment that will be effective.

For example, expression of CCL22, CCR9, POLR2C, FGFR1OP, PDE7A, DTYMK, ARPC1A, RPLP2, ERCC1, U2AF1, SF3B3, PRPF6, CDC25B, MYLK3, CCT4 and CCL11 can be determined by measuring expression of a nucleic acid molecule comprising at least 80%, at least 85%, at least 90%, at least 95%, at least 96%, at least 97%, at least 98%, at least 99%, or 100% sequence identity to SEQ ID NO: 1, 2, 3, 4, 5, 6, 7, 8, 9, 10, 11, 12, 13, 14, 20 or 21, respectively for example by using probes or primers that can specifically hybridize to such sequences or the complementary strand thereof. Similarly, expression of CCL22, CCR9, POLR2C, FGFR1OP, PDE7A, DTYMK, ARPC1A, RPLP2, ERCC1, U2AF1, SF3B3, PRPF6, CDC25B, MYLK3, LSM7, GABRA1, SLC44A4, RPL14, PFDN1, CCT4 and CCL11 can be determined by measuring expression of a protein encoded by a sequence comprising at least 80%, at least 85%, at least 90%, at least 95%, at least 96%, at least 97%, at least 98%, at least 99%, or 100% sequence identity to SEQ ID NO: 1, 2, 3, 4, 5, 6, 7, 8, 9, 10, 11, 12, 13, 14, 20 or 21, respectively, for example by using antibodies or fragments thereof that can specifically bind to such a protein.

For example, methylation of CCL22, CCR9, POLR2C, FGFR1OP, RPLP2, CDC25B, MYLK3, LSM7, GABRA1, SLC44A4, RPL14, PFDN1, CCT4 and CCL11 can be determined by measuring methylation of a nucleic acid molecule comprising at least 80%, at least 85%, at least 90%, at least 95%, at least 96%, at least 97%, at least 98%, at least 99%, or 100% sequence identity to SEQ ID NO: 1, 2, 3, 4, 8, 13, 14, 15, 16, 17, 18, 19, 20 or 21, respectively, for example by using probes or primers that can specifically hybridize to such sequences or the complementary strand thereof (for example primers or probes for bisulfite sequencing or conversion or pyrosequencing).

In some examples, a cancer that will, or is likely to, respond positively to chemotherapy, is one that when treated with one or more chemotherapeutic agents (e.g., carboplatin+paclitaxel, cisplatin+vinorelbine, or FOLFOX), reduces the size of a solid cancer (such as the volume or weight of a tumor), for example by at least 20%, at least 50%, at least 80%, at least 90%, at least 95%, at least 98%, or even at least 100%, as compared to the size of a cancer (such as the volume or weight of a tumor) in the absence of the chemotherapy. In contrast, a cancer that will not, or is likely not to, respond positively to chemotherapy, is one that when treated with one or more chemotherapeutic agents (e.g., carboplatin+paclitaxel, cisplatin+vinorelbine, or FOLFOX), does not significantly reduce the size of a solid cancer (such as the volume or weight of a tumor), for example reduces the size of a solid cancer by no more than 5%, no more than 1%, or no more than 0.1% (such as 0-5%), as compared to the size of a cancer (such as the volume or weight of a tumor) in the absence of the chemotherapy. In some examples, a cancer that will, or is likely to, respond positively to chemotherapy, is one that when treated with one or more chemotherapeutic agents (e.g., carboplatin+paclitaxel, cisplatin+vinorelbine, or FOLFOX), reduces the size of a cancer metastasis (such as the volume or weight of a metastasis, or number of metastases at a site distant from the primary tumor or cancer), for example by at least 20%, at least 50%, at least 80%, at least 90%, at least 95%, at least 98%, or even at least 100%, as compared to the size of a cancer metastasis in the absence of the chemotherapy. In contrast, a cancer that will not, or is likely not to, respond positively to chemotherapy, is one that when treated with one or more chemotherapeutic agents (e.g., carboplatin+paclitaxel, cisplatin+vinorelbine, or FOLFOX), does not significantly reduce the size of a cancer metastasis (such as the volume or weight of a metastasis, or number of metastases at a site distant from the primary tumor or cancer), for example reduces the size of a metastasis by no more than 5%, no more than 1%, or no more than 0.1% (such as 0-5%), as compared to the size of the metastasis (such as the volume or weight of a metastasis or number of metastases) in the absence of the chemotherapy. In some examples, a cancer that will, or is likely to, respond positively to chemotherapy, is one that when treated with one or more chemotherapeutic agents (e.g., carboplatin+paclitaxel, cisplatin+vinorelbine, or FOLFOX), increases the survival time of a patient with a cancer (such as LUAD, LUSC, or COAD), for example by at least 20%, at least 50%, at least 80%, at least 90%, at least 95%, at least 98%, or even at least 100%, as compared to the survival time in the absence of the chemotherapy. In contrast, a cancer that will not, or is likely not to, respond positively to chemotherapy, is one that when treated with one or more chemotherapeutic agents (e.g., carboplatin+paclitaxel, cisplatin+vinorelbine, or FOLFOX), does not significantly increase survival time of the treated patient, for example survival time by no more than 5%, no more than 1%, or no more than 0.1% (such as 0-5%), as compared to the survival time in the absence of the chemotherapy. In one example, the survival time of a patient with cancer that responds to chemotherapy is increased by at least 3 months, at least 4 months, at least 6 months, at least 8 months, at least 12 months, at least 24 months, at least 36 months, or at least 48 months, relative to patients with the same type of cancer who did not respond to the chemotherapy treatment (or did not receive the chemotherapy). In some examples, a cancer that will, or is likely to, respond positively to chemotherapy, is one that when treated with one or more chemotherapeutic agents (e.g., carboplatin+paclitaxel, cisplatin+vinorelbine, or FOLFOX), does not develop significant resistance to the chemotherapy, such as local or distant metastasis or cancer-related lethality, for example within one year of starting treatment with the chemotherapy, such as a reduction of local or distant metastasis or cancer-related lethality by at least 50%, at least 65%, at least 75%, at least 85%, at least 90%, at least 95%, or even at least 98% within one year of starting treatment with the chemotherapy, as compared to a subject that develops resistance to the same chemotherapy. In contrast, a cancer that will not, or is likely not to, respond positively to chemotherapy, is one that when treated with one or more chemotherapeutic agents (e.g., carboplatin+paclitaxel, cisplatin+vinorelbine, or FOLFOX), does develop resistance to the chemotherapy, for example within one year of starting treatment with the chemotherapy increases local or distant metastasis or cancer-related lethality by at least 50%, at least 65%, at least 75%, at least 85%, at least 90%, at least 95%, at least 98%, or 100% within one year of starting treatment with the chemotherapy, as compared to a subject that does not develop resistance to the same chemotherapy. In some examples, combinations of these affects are achieved. In some examples, a subject likely to develop chemotherapy resistance is one who will likely have a treatment-related relapse free survival (tRFS) of less than one year, that is the interval between chemotherapy administration (e.g., immediately after surgery) and the earliest relapse (e.g., as local, regional, or distant metastasis) will be within one year. Thus, in some examples, a subject who will develop resistance to chemotherapy is one who has a recurrence of their cancer within one year of treatment with the chemotherapy. In some examples, a subject not likely to develop chemotherapy resistance is one who will likely have a treatment-related relapse free survival (tRFS) of more than one year (if at all), that is the interval between chemotherapy administration (e.g., immediately after surgery) and the earliest relapse, if any, (e.g., as local, regional, or distant metastasis) will be after one year. Thus, in some examples, a subject who will not develop resistance to chemotherapy is one who does not have a recurrence of their cancer within one year of treatment with the chemotherapy.

Examples of methods for treating a subject with cancer or identifying a subject with cancer who responds positively to chemotherapy are disclosed herein (such as a subject with cancer that will be treated by the chemotherapy (such as a reduction in the size or metastasis of a tumor), and/or who does not develop resistance to chemotherapy). In some examples, the methods include measuring expression and/or methylation of cancer-related molecules from cancer-related pathways in a sample obtained from a subject (such as cancer sample, for example, a lung or colorectal cancer sample). A variety of molecules from various pathways can be measured. Further, the methods can include measuring any number of molecules. For example, at least about two, at least about three, at least about four, at least about five, at least about six, at least about seven, at least about eight, at least about nine, at least about 10, at least about 15, at least about 20, at least about 25, at least about 50, at least about 100, at least about 200, at least about 500, or at least about 1000, or about 2-5, about 2 to 7, about 2-10, about 1-25, about 10-50, about 25-100, about 100-500, or about 100-1000, or about 3, 5, 6, or 7 molecules can be measured. In some examples, molecules from at least about two, at least about three, at least about four, at least about five, at least about six, at least about seven, at least about eight, at least about nine, at least about 10, at least about 15, at least about 20, at least about 25, at least about 50, at least about 100, at least about 200, at least about 500, or at least about 1000, or about 3-5, about 3-7, about 3-10, about 3-25, about 10-50, about 25-100, about 100-500, or about 100-1000, or about 3, 5, 6, or 7 pathways can be measured.

The methods herein can further include comparing the expression and/or methylation of cancer-related molecules (such as lung or colorectal cancer-related molecules, such as DNA and mRNA respectively) from cancer-related pathways (such as lung or colorectal cancer-related pathways) measured in a sample obtained from a subject. In some examples, the measured expression and/or methylation of cancer-related molecules (such as lung or colorectal cancer-related molecules, such as DNA and mRNA respectively) from cancer-related pathways (such as lung or colorectal cancer-related pathways) are similar to the expression and/or methylation of cancer-related molecules (such as lung or colorectal cancer-related molecules, such as DNA and mRNA respectively) in a control representing expression and/or methylation for the cancer-related molecules (such as lung or colorectal cancer-related molecules, such as DNA and mRNA respectively) expected in a cancer from a subject that positively responds to a chemotherapy (such as a subject with cancer that will be treated by the chemotherapy (such as a reduction in the size or metastasis of a tumor), and/or who does not develop resistance to chemotherapy). Where such similar expression and/or methylation is measured, the subject can be identified as a subject who has a cancer that responds positively to chemotherapy. In some examples, the measured expression and/or methylation of cancer-related molecules (such as lung or colorectal cancer-related molecules, such as DNA and mRNA respectively) from cancer-related pathways (such as lung or colorectal cancer-related pathways) are similar to the expression and/or methylation of cancer-related molecules (such as lung or colorectal cancer-related molecules, such as DNA and mRNA respectively) in a control representing expression for the cancer-related molecules (such as lung or colorectal cancer-related molecules, such as DNA and mRNA respectively) expected in a cancer from a subject that positively responds to a chemotherapy (such as a subject with cancer that will be treated by the chemotherapy (such as a reduction in the size or metastasis of a tumor), and/or who does not develop resistance to chemotherapy). Where such similar expression and/or methylation is measured, the subject can be identified as a subject who responds positively to chemotherapy. Conversely, where similar expression and/or methylation is not present, the subject can be identified as a subject who will not respond positively to chemotherapy (such as a subject with cancer that will not be treated by the chemotherapy (such as a reduction in the size or metastasis of a tumor), and/or who does develop resistance to chemotherapy).

In some examples, the measured expression and/or methylation of cancer-related molecules from cancer-related pathways (such as lung or colorectal cancer-related pathways) differ from the expression and/or methylation of cancer-related molecules (such as lung or colorectal cancer-related molecules, such as DNA and mRNA respectively) in a control representing expression for the cancer-related molecules (such as lung or colorectal cancer-related molecules) expected in a cancer from a subject that does not positively respond to a chemotherapy (such as a subject with cancer that will not be treated by the chemotherapy (such as a reduction in the size or metastasis of a tumor), and/or who does develop resistance to chemotherapy). Where such differential expression and/or methylation is measured, the subject can be identified as a subject who responds positively to chemotherapy. Conversely, where differential expression and/or methylation is not measured, the subject can be identified as a subject having a cancer that does not respond positively to chemotherapy (such as a subject with cancer that will not be treated by the chemotherapy (such as a reduction in the size or metastasis of a tumor), and/or who does develop resistance to chemotherapy). In some examples, the methods include administering chemotherapy to a subject identified as one having a cancer that will respond positively to chemotherapy (such as a subject with cancer that will be treated by the chemotherapy (such as a reduction in the size or metastasis of a tumor), and/or who does not develop resistance to chemotherapy), thereby treating the subject. In other examples, the methods include administering other types of cancer therapy (such as surgery, radiation therapy, targeted therapy, immunotherapy, or palliative care) to a subject identified as one who will not respond positively to chemotherapy, thereby treating the subject.

Table 1 provides a summary of the cancers and their specific chemotherapy treatments, along with the pathways, and specific molecules, whose expression and/or methylation can be analyzed to determine if the cancer will respond positively to the chemotherapy. For example, mRNA expression and/or DNA methylation can be measured or detected in a lung cancer or colorectal cancer sample, and the expression and/or methylation compared to a control (e.g., representing methylation and/or expression observed in a particular cancer that does not respond positively to the particular chemotherapy) to determine if the cancer analyzed will positively respond to a particular chemotherapy regimen (e.g., depending on whether there is an increase or decrease in expression and/or methylation as noted in the table).

TABLE 1
Exemplary cancers and chemotherapy with cancer-related
pathways and cancer-related molecules with similar or
differential expression or methylation in a subject who
responds positively compared with subjects who do
not respond positively to chemotherapy.

| | | | Expression and/or Methylation | |
|---|---|---|---|---|
| | | | of cancer-related molecules in | |
| | | | subjects who respond | |
| | | | positively to chemotherapy | |
| Cancer | | | (compared with subjects who | |
| types & | Cancer- | Cancer- | do not respond positively) | |
| chemo- | related | related | mRNA | DNA |
| therapy | pathways | molecules | Expression | Methylation |
| LUAD_ | chemokine | CCL22 | decrease by | increase by |
| CP | receptors | | at least 50%, | at least |
| | bind | | at least 100%, | 50%, at |
| | chemokines | | at least | least 100%, |
| | | | 125%, such | at least |
| | | | as at least | 125%, such |
| | | | 68% (such as | as at |
| | | | by 68%) in | least 136% |
| | | | a LUAD that | (such as |
| | | | will respond | by 136%) in |
| | | | to carboplatin | a LUAD |
| | | | and paclitaxel | that will |
| | | | combination | respond to |
| | | | chemo- | carboplatin |
| | | | therapy, as | and |
| | | | compared | paclitaxel |
| | | | to CCL22 | combination |
| | | | expression | chemo- |
| | | | in a LUAD | therapy, as |
| | | | that will not | compared |
| | | | respond to | to CCL22 |
| | | | carboplatin | methylation |
| | | | and paclitaxel | in a |
| | | | combination | LUAD that |
| | | | chemotherapy | will not |
| | | | | respond to |
| | | | | carboplatin |
| | | | | and |
| | | | | paclitaxel |
| | | | | combination |
| | | | | chemotherapy |
| | mRNA | POLR2C | increase by | decrease by |
| | splicing | | at least 10%, | at least |
| | | | at least 20%, | 25%, at least |
| | | | at least | 50%, at |
| | | | 25%, such | least 70%, |
| | | | as at least | at least |
| | | | 26%, or at | 71% (such |
| | | | least 30% | as by |
| | | | (such as by | 71%) in |
| | | | 30%) in a | a LUAD |
| | | | LUAD that | that will |
| | | | will respond | respond to |
| | | | to carboplatin | carboplatin |
| | | | and paclitaxel | and |
| | | | combination | paclitaxel |
| | | | chemo- | combination |
| | | | therapy, as | chemo- |
| | | | compared to | therapy, as |
| | | | POLR2C | compared to |
| | | | expression in | POLR2C |
| | | | a LUAD | methylation |
| | | | that will not | in a |
| | | | respond to | LUAD that |
| | | | carboplatin | will not |
| | | | and paclitaxel | respond to |
| | | | combination | carboplatin |
| | | | chemotherapy | and paclitaxel |
| | | | | combination |
| | | | | chemotherapy |
| | G alpha (s) | PDE7A | decrease by | |
| | signalling | | at least 15%, | |
| | events | | at least 20%, | |
| | | | at least | |
| | | | 30%, such | |
| | | | as at least | |
| | | | 32% (such as | |
| | | | by 32%) in | |
| | | | a LUAD that | |
| | | | will respond to | |
| | | | carboplatin | |
| | | | and paclitaxel | |
| | | | combination | |
| | | | chemotherapy, | |
| | | | as compared | |
| | | | to PDE7A | |
| | | | expression | |
| | | | in a lung | |
| | | | adeno- | |
| | | | carcinoma | |
| | | | that will | |
| | | | not respond to | |
| | | | carboplatin and | |
| | | | paclitaxel | |
| | | | combination | |
| | | | chemotherapy | |
| | intestinal | CCR9 | decrease by | increase by |
| | immune | | at least 5%, | at least |
| | network | | at least 8%, | 15%, at |
| | for IgA | | at least 10%, | least 20%, at |
| | production | | at least 15%, | least 30%, |
| | | | at least | such as at |
| | | | 20%, such | least 39% |
| | | | as at least | (such as |
| | | | 25% (such as | by 39%) |
| | | | by 8%) in a | in a LUAD |
| | | | LUAD that | that will |
| | | | will respond | respond to |
| | | | to carboplatin | carboplatin |
| | | | and paclitaxel | and paclitaxel |
| | | | combination | combination |
| | | | chemo- | chemotherapy, |
| | | | therapy, as | as compared |
| | | | compared | to CCR9 |
| | | | to CCR9 | methylation |
| | | | expression | in a LUAD |
| | | | in a LUAD | that will not |
| | | | that will not | respond to |
| | | | respond to | carboplatin and |
| | | | carboplatin and | paclitaxel |
| | | | paclitaxel | combination |
| | | | combination | chemotherapy |
| | | | chemotherapy | |
| | metabolism | CCT4 | increase by at | decrease by |
| | of proteins | | least 15%, | at least |
| | | | at least 20%, | 25%, at |
| | | | at least | least 50% at |
| | | | 30%, at least | least 60%, |
| | | | 50%, at | at least |
| | | | least 75%, at | 70%, (such |
| | | | least 90%, | as by 70%) |
| | | | such as at | in a LUAD |
| | | | least 92% | that will |
| | | | (such as | respond to |
| | | | by 92%) | carboplatin and |
| | | | in a LUAD that | paclitaxel |
| | | | will respond | combination |
| | | | to carboplatin | chemo- |
| | | | and paclitaxel | therapy, as |
| | | | combination | compared |
| | | | chemo- | to CCT4 |
| | | | therapy, as | methylation in a |
| | | | compared | LUAD that |
| | | | to CCT4 | will not |
| | | | expression in | respond to |
| | | | a LUAD | carboplatin |
| | | | that will not | and |
| | | | respond to | paclitaxel |
| | | | carboplatin and | combination |
| | | | paclitaxel | chemotherapy |
| | | | combination | |
| | | | chemotherapy | |
| | RNA | LSM7 | | decrease by |
| | degradation | | | at least |
| | | | | 25%, at least |
| | | | | 40%, at |
| | | | | least 50%, |
| | | | | at least |
| | | | | 70%, at |
| | | | | least 76%, |
| | | | | (such as by |
| | | | | 76%) in a |
| | | | | LUAD that will |
| | | | | respond to |
| | | | | carboplatin and |
| | | | | paclitaxel |
| | | | | combination |
| | | | | chemotherapy, |
| | | | | as compared |
| | | | | to LSM7 |
| | | | | methylation in a |
| | | | | LUAD that |
| | | | | will not |
| | | | | respond to |
| | | | | carboplatin and |
| | | | | paclitaxel |
| | | | | combination |
| | | | | chemotherapy |
| | cell cycle | FGFR1OP | increase by | decrease by |
| | mitotic | | at least 20%, | at least |
| | | | at least 25%, | 25%, at |
| | | | at least | least 40%, |
| | | | 40%, at least | at least |
| | | | 50%, at | 50%, at least |
| | | | least 57%, | 60%, at |
| | | | (such as by | least 65% (such |
| | | | 57%) in a | as by 65%) in |
| | | | LUAD | a LUAD that |
| | | | carcinoma that | will respond to |
| | | | will respond to | carboplatin |
| | | | carboplatin | and |
| | | | and paclitaxel | paclitaxel |
| | | | combination | combination |
| | | | chemotherapy, | chemo- |
| | | | as compared to | therapy, as |
| | | | FGFR1OP | compared to |
| | | | expression | FGFR1OP |
| | | | in a lung | methylation |
| | | | adeno- | in a |
| | | | carcinoma | LUAD that |
| | | | that will | will not |
| | | | not respond to | respond to |
| | | | carboplatin | carboplatin and |
| | | | and paclitaxel | paclitaxel |
| | | | combination | combination |
| | | | chemotherapy | chemotherapy |
| LUAD_ | metabolism | DTYMK | increase by | |
| CV | of | | at least 50%, | |
| | nucleotides | | at least 75%, | |
| | | | at least | |
| | | | 100%, such | |
| | | | as at least | |
| | | | 105% (such | |
| | | | as by 105%) | |
| | | | in a LUAD | |
| | | | that will | |
| | | | respond to | |
| | | | cisplatin and | |
| | | | vinorelbine | |
| | | | combination | |
| | | | chemotherapy, | |
| | | | as expression | |
| | | | in a lung | |
| | | | adeno- | |
| | | | carcinoma | |
| | | | that will | |
| | | | not respond | |
| | | | to cisplatin | |
| | | | compared to | |
| | | | DTYMK | |
| | | | and vinorelbine | |
| | | | combination | |
| | | | chemotherapy | |
| | actin Y | ARPC1A | increase by | |
| | | | at least 15%, | |
| | | | at least 20%, | |
| | | | at least | |
| | | | 25%, such | |
| | | | as at least | |
| | | | 30% (such as | |
| | | | by 30%) in | |
| | | | a LUAD | |
| | | | that will | |
| | | | respond to | |
| | | | cisplatin and | |
| | | | vinorelbine | |
| | | | combination | |
| | | | chemotherapy, | |
| | | | as compared | |
| | | | to ARPC1A | |
| | | | expression | |
| | | | in a LUAD | |
| | | | that will not | |
| | | | respond to | |
| | | | cisplatin and | |
| | | | vinorelbine | |
| | | | combination | |
| | | | chemotherapy | |
| | ribosome | RPLP2 | increase by | decrease by |
| | | | at least 20%, | at least |
| | | | at least 30%, | 1%, at least |
| | | | at least | 2.5%, at |
| | | | 40%, such | least 3%, |
| | | | as at least | at least |
| | | | 41% (such as | 4%, such |
| | | | by 41%) in | as at least |
| | | | a LUAD that | 5% (such |
| | | | will respond to | as by 5%) |
| | | | cisplatin and | in LUAD |
| | | | vinorelbine | that will |
| | | | combination | respond |
| | | | chemo- | to cisplatin |
| | | | therapy, as | and |
| | | | compared to | vinorelbine |
| | | | RPLP2 | combination |
| | | | expression | chemo- |
| | | | in a LUAD | therapy, as |
| | | | that will not | compared |
| | | | respond to | to RPLP2 |
| | | | cisplatin and | methylation |
| | | | vinorelbine | in a LUAD |
| | | | combination | that will not |
| | | | chemotherapy | respond to |
| | | | | cisplatin and |
| | | | | vinorelbine |
| | | | | combination |
| | | | | chemotherapy |
| LUSC | cytokine- | CCL11 | decrease by | increase by |
| | cytokine | | at least 25%, | at least 50%, |
| | receptor | | at least 40%, | at least 100%, |
| | interaction | | at least 50%, | at least 200%, |
| | | | at least 60%, at | at least 300%, |
| | | | least 61% | such as at least |
| | | | (such as by | 411% (such |
| | | | 61%) in LUSC | as by 411%) in |
| | | | a that will | LUSC that |
| | | | respond to | will respond |
| | | | cisplatin and | to cisplatin |
| | | | vinorelbine | and vinorelbine |
| | | | combination | combination |
| | | | chemo- | chemo- |
| | | | therapy, as | therapy, as |
| | | | compared | compared |
| | | | to CCL11 | to CCL11 |
| | | | expression | methylation |
| | | | in a LUSC | in a LUSC |
| | | | that will not | that will not |
| | | | respond to | respond to |
| | | | cisplatin and | cisplatin and |
| | | | vinorelbine | vinorelbine |
| | | | combination | combination |
| | | | chemotherapy | chemotherapy |
| | neuroactive | GABRA1 | | increase by at |
| | ligand- | | | least 50%, |
| | receptor | | | at least 100%, |
| | interaction | | | at least 200%, at |
| | | | | least 225%, |
| | | | | such as at |
| | | | | least 242% (such |
| | | | | as by 242%) in 1 |
| | | | | LUSC that will |
| | | | | respond to cisplatin |
| | | | | and vinorelbine |
| | | | | combination |
| | | | | chemotherapy, as |
| | | | | compared to |
| | | | | GABRA1 |
| | | | | methylation in a |
| | | | | LUSC that will not |
| | | | | respond to cisplatin |
| | | | | and vinorelbine |
| | | | | combination |
| | | | | chemotherapy |
| | DNA repair | ERCC1 | decrease by | |
| | | | at least 25%, | |
| | | | at least 30%, | |
| | | | at least 35%, | |
| | | | at least 40%, at | |
| | | | least 47% (such | |
| | | | as by 47%) | |
| | | | in LUSC that | |
| | | | will respond to | |
| | | | cisplatin and | |
| | | | vinorelbine | |
| | | | combination | |
| | | | chemotherapy, | |
| | | | as compared | |
| | | | to ERCC1 | |
| | | | expression | |
| | | | in a LUSC that | |
| | | | will not | |
| | | | respond to | |
| | | | cisplatin and | |
| | | | vinorelbine | |
| | | | combination | |
| | | | chemotherapy | |
| | SLC- | SLC44A4 | | increase by |
| | mediated | | | at least |
| | transport | | | 50%, at |
| | trans- | | | least 100%, at |
| | membrane | | | least 150%, at |
| | translation | | | least 175%, |
| | | | | such as at |
| | | | | least 185% |
| | | | | (such |
| | | | | as by 185%) in |
| | | | | LUSC that will |
| | | | | respond to cisplatin |
| | | | | and vinorelbine |
| | | | | combination |
| | | | | chemotherapy, as |
| | | | | compared to |
| | | | | SLC44A4 |
| | | | | methylation in a |
| | | | | LUSC that will not |
| | | | | respond to cisplatin |
| | | | | and vinorelbine |
| | | | | combination |
| | | | | chemotherapy |
| | translation | RPL14 | | decrease by |
| | | | | at least |
| | | | | 1%, at least |
| | | | | 2.5%, at |
| | | | | least 3%, |
| | | | | such as at |
| | | | | least 4% |
| | | | | (such as by |
| | | | | 4%) in LUSC that |
| | | | | will respond to |
| | | | | cisplatin and |
| | | | | vinorelbine |
| | | | | combination |
| | | | | chemotherapy, as |
| | | | | compared |
| | | | | to RPL14 |
| | | | | methylation in a |
| | | | | LUSC that will not |
| | | | | respond to cisplatin |
| | | | | and vinorelbine |
| | | | | combination |
| | | | | chemotherapy |
| | transport | U2AF1 | decrease by | |
| | of mature | | at least 5%, | |
| | mRNA | | at least 8%, | |
| | derived | | at least 10%, | |
| | from | | such as at | |
| | an intron- | | least 11% | |
| | containing | | (such as by | |
| | transcript | | 11%) in LUSC | |
| | | | that | |
| | | | will respond | |
| | | | to cisplatin and | |
| | | | vinorelbine | |
| | | | combination | |
| | | | chemotherapy, | |
| | | | as compared | |
| | | | to U2AF1 | |
| | | | expression in | |
| | | | a LUSC | |
| | | | that will | |
| | | | not respond to | |
| | | | cisplatin and | |
| | | | vinorelbine | |
| | | | combination | |
| | | | chemotherapy | |
| COAD | elongation | SF3B3 | increase by at | |
| | and | | least 10%, at | |
| | processing | | least 20%, at | |
| | of capped | | least 25%, such | |
| | transcripts | | as at least 29% | |
| | | | (such as by | |
| | | | 29%) in a | |
| | | | COAD | |
| | | | that will | |
| | | | respond to | |
| | | | FOLFOX | |
| | | | combination | |
| | | | chemotherapy, | |
| | | | as compared | |
| | | | to SF3B3 | |
| | | | expression in | |
| | | | a COAD that | |
| | | | will not | |
| | | | respond | |
| | | | to FOLFOX | |
| | | | combination | |
| | | | chemotherapy | |
| | processing | PRPF6 | increase by at | |
| | of capped | | least 25%, | |
| | intron | | at least 50%, | |
| | containing | | at least 60%, | |
| | pre mRNA | | such as at least | |
| | | | 63% (such as | |
| | | | by 63%) in | |
| | | | a COAD that | |
| | | | will respond | |
| | | | to FOLFOX | |
| | | | combination | |
| | | | chemotherapy, | |
| | | | as compared | |
| | | | to PRPF6 | |
| | | | expression in | |
| | | | a COAD that | |
| | | | will not | |
| | | | respond | |
| | | | to FOLFOX | |
| | | | combination | |
| | | | chemotherapy | |
| | metabolism | PFDN1 | | decrease by |
| | of protein | | | at least |
| | | | | 20%, at |
| | | | | least 25%, at |
| | | | | least 29% |
| | | | | (such as by |
| | | | | 29%) in |
| | | | | a COAD |
| | | | | that will |
| | | | | respond to |
| | | | | FOLFOX |
| | | | | combination |
| | | | | chemo- |
| | | | | therapy, as |
| | | | | compared |
| | | | | to PFDN1 |
| | | | | methylation |
| | | | | in a COAD |
| | | | | that will not |
| | | | | respond to |
| | | | | FOLFOX |
| | | | | combination |
| | | | | chemotherapy |
| | 5 phase | CDC25B | increase by | decrease by |
| | | | at least 10%, | at least 25%, |
| | | | at least 20%, | at least 40%, at |
| | | | at least 25%, | least 50%, |
| | | | such as at least | at least |
| | | | 31% (such as | 60%, at |
| | | | by 31%) in | least 63% |
| | | | a COAD | (such as |
| | | | that will | by 63%) in |
| | | | respond to | a COAD |
| | | | FOLFOX | that will |
| | | | combination | respond to |
| | | | chemo- | FOLFOX |
| | | | therapy, as | combination |
| | | | compared | chemo- |
| | | | to CDC25B | therapy, as |
| | | | expression | compared to |
| | | | in a COAD | CDC25B |
| | | | that will not | methylation |
| | | | respond to | in a COAD |
| | | | FOLFOX | that will not |
| | | | combination | respond to |
| | | | chemotherapy | FOLFOX |
| | | | | combination |
| | | | | chemotherapy |
| | calcium | MYLK3 | decrease by | increase by |
| | signaling | | at least 25%, | at least |
| | | | at least 40%, | 50%, at least |
| | | | at least 50%, | 75%, at |
| | | | at least 57%, | least 80%, |
| | | | (such as by | such as at |
| | | | 57%) in a | least 81% |
| | | | COAD that | (such as |
| | | | will respond to | by 81%) |
| | | | FOLFOX | in a COAD |
| | | | combination | that will |
| | | | chemo- | respond to |
| | | | therapy, as | FOLFOX |
| | | | compared to | combination |
| | | | MYLK3 | chemo- |
| | | | expression | therapy, as |
| | | | in a COAD | compared to |
| | | | that will not | MYLK3 |
| | | | respond to | methylation |
| | | | FOLFOX | in a COAD |
| | | | combination | that will |
| | | | chemotherapy | not respond to |
| | | | | FOLFOX |
| | | | | combination |
| | | | | chemotherapy |

Note:
LUAD_CP = lung adenocarcinoma treated with carboplatin and paclitaxel;
LUAD_CV = lung adenocarcinoma treated with cisplatin and vinorelbine;
LUSC = lung squamous cell carcinoma treated with cisplatin and vinorelbine;
COAD = Colon Adenocarcinoma treated with FOLFOX (folinic acid, fluorouracil, oxaliplatin).

Evaluating Expression and Methylation in Subjects with Lung Cancer

The methods disclosed herein can be used to treat subjects with lung cancer or identify subjects with lung cancer who respond positively to chemotherapy, that is, have a cancer that will be effectively treated by the chemotherapy. In some examples, the methods include measuring expression and/or methylation of lung cancer-related molecules (e.g., mRNAs) from lung cancer-related pathways in a sample (such as a lung cancer sample) obtained from a subject, such as a subject with lung cancer. Various lung cancer-related pathways are possible. Exemplary lung cancer-related pathways include chemokine receptor, mitotic cell cycle, immune network for immunoglobulin A (IgA) production, RNA degradation, mRNA splicing, protein metabolism, G alpha signaling, nucleotide metabolism, actin Y, ribosome pathways, cytokine-cytokine receptor interaction, neuroactive ligand-receptor interaction, DNA repair, SLC-mediated transmembrane transport, translation, and transport of mature mRNA derived from an intron-containing transcript pathways. In specific, non-limiting examples, the lung cancer-related pathways can include chemokine receptor, mitotic cell cycle, immune network for immunoglobulin A (IgA) production, RNA degradation, mRNA splicing, protein metabolism, and G alpha signaling pathways; nucleotide metabolism, actin Y, and ribosome pathways; or cytokine-cytokine receptor interaction, neuroactive ligand-receptor interaction, DNA repair, SLC-mediated transmembrane transport, translation, and transport of mature mRNA derived from an intron-containing transcript pathways.

A variety of lung cancer-related molecules are possible. Exemplary lung cancer-related molecules include C-C motif chemokine ligand 22 (CCL22; for example, from the chemokine receptor pathway), fibroblast growth factor receptor 1 oncogene partner (FGFR1OP; for example, from the mitotic cell cycle pathway), C-C motif chemokine receptor 9 (CCR9; for example, from the immune network for IgA production and chemokine receptor pathway), LSM7 (for example; from the RNA degradation pathway), RNA polymerase II subunit C (POLR2C; for example, from the RNA splicing pathway, chaperonin containing TCP1 subunit 4 (CCT4; for example, from the protein metabolism pathway, and phosphodiesterase 7A (PDE7A; for example, from the G alpha signaling pathway, deoxythymidylate kinase (DTYMK; for example, from the nucleotide metabolism pathway), actin-related protein 2/3 complex subunit 1A (ARPC1A; for example, from the actin Y pathway), ribosomal protein lateral stalk subunit P2 (RPLP2; for example, from the ribosome pathway), C-C motif chemokine 11 (CCL11; for example, from the cytokine-cytokine receptor interaction pathway), gamma-aminobutyric acid receptor alpha-1 (GABRA1; for example, from the neuroactive ligand-receptor interaction pathway), excision repair cross-complementation group 1 (ERCC1; for example, from the DNA repair pathway), solute carrier family 44 member 4 (SLC44A4; for example, from the solute carrier (SLC)-mediated transmembrane transport pathway), ribosomal protein L14 (RPL14; for example, from the translation pathway), and U2 small nuclear RNA auxiliary factor 1 (U2AF1; for example, from the transport of mature mRNA derived from an intron-containing transcript pathway).

Evaluating Expression and Methylation in Subjects with Lung Adenocarcinoma

In specific, non-limiting examples, the methods can be used to treat subjects with lung adenocarcinoma (LUAD) or identify subjects with a LUAD that will respond positively to chemotherapy (such as a subject with LUAD that will be treated by the chemotherapy), such as a reduction in the size or metastasis of the LUAD, and/or who does not develop resistance to chemotherapy. The methods can include measuring expression and/or methylation of LUAD-related molecules from LUAD-related pathways in a sample (such as a lung cancer sample) obtained from a subject with LUAD. Exemplary LUAD-related pathways include chemokine receptor, mitotic cell cycle, immune network for IgA production, RNA degradation, mRNA splicing, protein metabolism, G alpha signaling, nucleotide metabolism, actin Y, and ribosome pathways. Exemplary LUAD-related molecules include CCL22, CCR9, POLR2C, FGFR1OP, CCT4, LSM7 DTYMK, ARPC1A, and RPLP2 mRNA and DNA molecules. The methods herein can further include comparing the expression and/or methylation of LUAD-related molecules from LUAD-related pathways measured in a sample obtained from a subject. In some examples, the measured expression and/or methylation of LUAD-related molecules from LUAD-related pathways is compared with the expression and/or methylation of LUAD-related molecules in a control representing expression for the LUAD-related molecules expected in a LUAD sample that (1) does not positively respond to a chemotherapy (such as a LUAD that will not be treated by the chemotherapy, such as no significant reduction in the size or metastasis of the LUAD, and/or the LUAD develops resistance to chemotherapy) or (2) does positively respond to chemotherapy, such as a LUAD that will be treated by the chemotherapy, such as a reduction in the size or metastasis of the LUAD and/or does not develop resistance to chemotherapy. Where differential expression and/or methylation is measured compared with a control representing expression and/or methylation for the LUAD-related molecules expected in a sample from a LUAD that does not positively respond to a chemotherapy, or similar expression and/or methylation is measured compared with a control representing expression and/or methylation expected in a LUAD that does positively respond to chemotherapy, the subject with such an LUAD can be identified as a subject who responds positively to chemotherapy (such as a subject with LUAD that will be treated by the chemotherapy, such as a reduction in the size or metastasis of the LUAD, and/or who does not develop resistance to chemotherapy). Conversely, where similar expression and/or methylation is measured compared with a control representing expression and/or methylation for the LUAD-related molecules expected in a LUAD sample that does not positively respond to a chemotherapy, or differential expression and/or methylation is measured compared with a control representing expression and/or methylation expected in a LUAD sample that does positively respond to chemotherapy, the subject can be identified as a subject having a LUAD that does not respond positively to chemotherapy (such as a subject with LUAD that will not be treated by the chemotherapy (such as a reduction in the size or metastasis of the LUAD), and/or who does develop resistance to chemotherapy). In some examples, the methods include administering chemotherapy (such as carboplatin, paclitaxel, cisplatin, vinorelbine, or a combination thereof) to a subject identified as one who responds positively to chemotherapy, thereby treating the LUAD in the subject. In other examples, the methods include administering other types of LUAD therapy (such as surgery, radiation therapy, targeted therapy, immunotherapy, or palliative care) to a subject identified as one who does not responds positively to chemotherapy, thereby treating the subject.

In some examples the method identifies subjects having a LUAD that respond positively to the chemotherapy combination carboplatin and paclitaxel. The methods can include measuring expression and/or methylation of LUAD-related molecules (such as mRNA and DNA, respectively) from chemokine receptor, mitotic cell cycle, immune network for IgA production, RNA degradation, mRNA splicing, protein metabolism, and G alpha signaling pathways in a sample obtained from a subject with LUAD. In some examples, the LUAD-related molecules include CCL22, CCR9, POLR2C, LSM7, FGFR1OP, PDE7A, and CCT4 mRNA and DNA. Where differential expression (such as expression of LUAD-related molecules from chemokine receptor, mitotic cell cycle, immune network for IgA production, mRNA splicing, protein metabolism, and G alpha signaling pathways or CCL22, CCR9, POLR2C, FGFR1OP, CCT4, and PDE7A) and/or methylation (such as methylation of LUAD-related molecules from chemokine receptor, mitotic cell cycle, immune network for IgA production, mRNA splicing, protein metabolism, and RNA degradation pathways or CCL22, CCR9, POLR2C, FGFR1OP, CCT4, and LSM7) is measured compared with a control representing expression and/or methylation for the LUAD-related molecules expected in a LUAD sample that does not positively respond to the chemotherapy combination carboplatin and paclitaxel, or similar expression (such as expression of LUAD-related molecules from chemokine receptor, mitotic cell cycle, immune network for IgA production, mRNA splicing, protein metabolism, and G alpha signaling pathways or CCL22, CCR9, POLR2C, FGFR1OP, CCT4, and PDE7A) and/or methylation (such as methylation of LUAD-related molecules from chemokine receptor, mitotic cell cycle, immune network for IgA production, mRNA splicing, protein metabolism, and RNA degradation pathways or CCL22, CCR9, POLR2C, FGFR1OP, CCT4, and LSM7) is measured compared with a control representing expression and/or methylation expected in a LUAD sample that does positively respond to the chemotherapy combination carboplatin and paclitaxel, the subject can be identified as a subject having a LUAD that will respond positively to chemotherapy (i.e., carboplatin and paclitaxel). Conversely, where similar expression (such as expression of LUAD-related molecules from chemokine receptor, mitotic cell cycle, immune network for IgA production, mRNA splicing, protein metabolism, and G alpha signaling pathways or CCL22, CCR9, POLR2C, FGFR1OP, CCT4, and PDE7A) and/or methylation (such as methylation of LUAD-related molecules from chemokine receptor, mitotic cell cycle, immune network for IgA production, mRNA splicing, protein metabolism, and RNA degradation pathways or CCL22, CCR9, POLR2C, FGFR1OP, CCT4, and LSM7) is measured compared with a control representing expression and/or methylation for the LUAD-related molecules (such as mRNA and DNA, respectively) expected in a LUAD that does not positively respond to the chemotherapy combination carboplatin and paclitaxel, or differential expression (such as expression of LUAD-related molecules from chemokine receptor, mitotic cell cycle, immune network for IgA production, mRNA splicing, protein metabolism, and G alpha signaling pathways or CCL22, CCR9, POLR2C, FGFR1OP, CCT4, and PDE7A) and/or methylation (such as methylation of LUAD-related molecules from chemokine receptor, mitotic cell cycle, immune network for IgA production, mRNA splicing, protein metabolism, and RNA degradation pathways or CCL22, CCR9, POLR2C, FGFR1OP, CCT4, and LSM7) is measured compared with a control representing expression and/or methylation expected in a LUAD that does positively respond to the chemotherapy combination carboplatin and paclitaxel, the subject can be identified as a subject having a LUAD that does not respond positively to chemotherapy (i.e., carboplatin and paclitaxel). In some examples, the methods include administering the chemotherapy combination carboplatin and paclitaxel to a subject identified as one who responds positively to chemotherapy (i.e., carboplatin and paclitaxel), thereby treating the LUAD in the subject. In other examples, the methods include administering other types of LUAD therapy (such as surgery, radiation therapy, targeted therapy, immunotherapy, or palliative care) to a subject identified as one who does not responds positively to chemotherapy, thereby treating the subject.

In some examples, the methods identify subjects with LUAD that respond positively to the chemotherapy combination cisplatin and vinorelbine. The methods can include measuring expression (such as expression of LUAD-related molecules from nucleotide metabolism, actin Y, and ribosome pathways or DTYMK, ARPC1A, and RPLP2) and/or methylation (such as methylation of LUAD-related molecules from the ribosome pathway or RPLP2) in a LUAD sample obtained from a subject. Where differential expression (such as expression of LUAD-related mRNA molecules from nucleotide metabolism, actin Y, and ribosome pathways or DTYMK, ARPC1A, and RPLP2) and/or methylation (such as methylation of LUAD-related DNA molecules from the ribosome pathway or RPLP2) is measured compared with a control representing expression and/or methylation for the LUAD-related molecules expected in a LUAD that does not positively respond to the chemotherapy combination cisplatin and vinorelbine, or similar expression and/or methylation is measured compared with a control representing expression and/or methylation expected in a LUAD that does positively respond to the chemotherapy combination cisplatin and vinorelbine, the subject can be identified as one having a LUAD that responds positively to chemotherapy (i.e., cisplatin and vinorelbine). Conversely, where similar expression (such as expression of LUAD-related molecules from nucleotide metabolism, actin Y, and ribosome pathways or DTYMK, ARPC1A, and RPLP2) and/or methylation (such as methylation of LUAD-related molecules from the ribosome pathway or RPLP2) is measured compared with a control representing expression and/or methylation for the LUAD-related molecules expected in a sample from a subject who does not positively respond to the chemotherapy combination cisplatin and vinorelbine, or differential expression and/or methylation is measured compared with a control representing expression and/or methylation expected in a sample for a subject who does positively respond to the chemotherapy combination cisplatin and vinorelbine, the subject can be identified as a subject who does not respond positively to chemotherapy (i.e., cisplatin and vinorelbine). In some examples, the methods include administering the chemotherapy combination cisplatin and vinorelbine to a subject identified as one who responds positively to cisplatin and vinorelbine, thereby treating the subject. In other examples, the methods include administering other types of LUAD therapy (such as surgery, radiation therapy, targeted therapy, immunotherapy, or palliative care) to a subject identified as one who does not responds positively to chemotherapy (i.e., cisplatin and vinorelbine), thereby treating the subject.

Evaluating Expression and Methylation in a Subject with Lung Squamous Cell Carcinoma

In some examples, the methods are used to treat subjects with lung squamous cell carcinoma (LUSC) or identify subjects with LUSC that respond positively to chemotherapy (such as cisplatin and vinorelbine). The methods can include measuring expression and/or methylation of LUSC-related molecules from LUSC-related pathways in a sample (such as a lung cancer sample) obtained from a subject with LUSC. Exemplary LUSC-related pathways include cytokine-cytokine receptor interaction, neuroactive ligand-receptor interaction, DNA repair, SLC-mediated transmembrane transport, translation, and transport of mature mRNA derived from an intron-containing transcript pathways. Exemplary LUSC-related molecules include CCL11, GABRA1, ERCC1, SLC44A4, RPL14, and U2AF1.

The methods can further include comparing the expression and/or methylation of LUSC-related molecules (e.g., mRNA and DNA, respectively) from LUSC-related pathways measured in a sample obtained from a subject. In some examples, the measured expression (such as expression of LUSC-related molecules from cytokine-cytokine receptor interaction, DNA repair, and transport of mature mRNA derived from an intron-containing transcript pathways or CCL11, ERCC1, and U2AF1) and/or methylation (such as methylation of LUSC-related molecules from cytokine-cytokine receptor interaction, neuroactive ligand-receptor interaction, SLC-mediated transmembrane transport, and translation pathways or CCL11, GABRA1, SLC44A4, and RPL14) of LUSC-related molecules from LUSC-related pathways is compared with the expression (such as expression of LUSC-related molecules from cytokine-cytokine receptor interaction, DNA repair, and transport of mature mRNA derived from an intron-containing transcript pathways or CCL11, ERCC1, and U2AF1) and/or methylation (such as methylation of LUSC-related molecules from cytokine-cytokine receptor interaction, neuroactive ligand-receptor interaction, SLC-mediated transmembrane transport, and translation pathways or CCL11, GABRA1, SLC44A4, and RPL14) of LUSC-related molecules in a control representing expression for the LUSC-related molecules expected in a LUSC that (1) does not positively respond to a chemotherapy (such as a LUSC that will not be treated by cisplatin and vinorelbine, such as no significant reduction in the size or metastasis of the LUSC, and/or a LUSC that develops resistance to cisplatin and vinorelbine) or (2) does positively respond to chemotherapy (such as a subject with LUSC that will be treated by the cisplatin and vinorelbine, such as a reduction in the size or metastasis of the tumor, and/or who does not develop resistance to cisplatin and vinorelbine.

Where differential expression (such as expression of LUSC-related molecules from cytokine-cytokine receptor interaction, DNA repair, and transport of mature mRNA derived from an intron-containing transcript pathways or CCL11, ERCC1, and U2AF1) and/or methylation (such as methylation of LUSC-related molecules (e.g., mRNA and DNA, respectively) from cytokine-cytokine receptor interaction, neuroactive ligand-receptor interaction, SLC-mediated transmembrane transport, and translation pathways or CCL11, GABRA1, SLC44A4, and RPL14) is measured compared with a control representing expression and/or methylation for the LUSC-related molecules expected in a LUSC that does not positively respond to a chemotherapy (i.e., cisplatin and vinorelbine), or similar expression (such as expression of LUSC-related molecules from cytokine-cytokine receptor interaction, DNA repair, and transport of mature mRNA derived from an intron-containing transcript pathways or CCL11, ERCC1, and U2AF1) and/or methylation (such as methylation of LUSC-related molecules from cytokine-cytokine receptor interaction, neuroactive ligand-receptor interaction, SLC-mediated transmembrane transport, and translation pathways or CCL11, GABRA1, SLC44A4, and RPL14) is measured compared with a control representing expression and/or methylation expected in a LUSC that does positively respond to chemotherapy (i.e., cisplatin and vinorelbine), the subject can be identified as a subject having a LUSC that responds positively to chemotherapy (i.e., cisplatin and vinorelbine).

Conversely, where similar expression (such as expression of LUSC-related molecules from cytokine-cytokine receptor interaction, DNA repair, and transport of mature mRNA derived from an intron-containing transcript pathways or CCL11, ERCC1, and U2AF1) and/or methylation (such as methylation of LUSC-related molecules from cytokine-cytokine receptor interaction, neuroactive ligand-receptor interaction, SLC-mediated transmembrane transport, and translation pathways or CCL11, GABRA1, SLC44A4, and RPL14) is measured compared with a control representing expression and/or methylation for the LUSC-related molecules expected in a sample from a LUSC that does not positively respond to a chemotherapy (i.e., cisplatin and vinorelbine), or differential expression (such as expression of LUSC-related molecules from cytokine-cytokine receptor interaction, DNA repair, and transport of mature mRNA derived from an intron-containing transcript pathways or CCL11, ERCC1, and U2AF1) and/or methylation (such as methylation of LUSC-related molecules from cytokine-cytokine receptor interaction, neuroactive ligand-receptor interaction, SLC-mediated transmembrane transport, and translation pathways or CCL11, GABRA1, SLC44A4, and RPL14) is measured compared with a control representing expression and/or methylation expected in a LUSC that does positively respond to chemotherapy, the subject can be identified as a subject having a LUSC that does not respond positively to chemotherapy.

In some examples, the methods include administering chemotherapy (such as cisplatin and vinorelbine) to a subject identified as one who responds positively to cisplatin and vinorelbine, thereby treating the LUSC in the subject. In other examples, the methods include administering other types of LUSC therapy (such as surgery, radiation therapy, targeted therapy, immunotherapy, or palliative care) to a subject identified as one who does not respond positively to cisplatin and vinorelbine, thereby treating the subject.

Evaluating Expression and Methylation in Subjects with Colon Cancer

The methods disclosed herein can be used to treat subjects with colon cancer (such as colon adenocarcinoma (COAD)) or identify subjects with colon cancer (such as COAD) that respond positively to chemotherapy (such as folinic acid, fluorouracil, oxaliplatin, (FOLFOX) or a combination thereof). The methods can include measuring expression and/or methylation of colon cancer-related molecules (such as COAD-related molecules, such as mRNA and/or DNA, respectively) from colon cancer-related pathways (such as COAD-related pathways) in a sample (such as a colon cancer sample) obtained from a subject with colon cancer (such as COAD). Exemplary colon cancer-related pathways (such as COAD-related pathways) include elongation and processing of capped transcripts, processing of capped intron containing pre-mRNA, protein metabolism, S phase, and calcium signaling pathways. Exemplary colon cancer-related molecules (such as COAD-related molecules) include splicing factor 3b subunit 3 (SF3B3), pre-mRNA processing factor 6 (PRPF6), prefoldin subunit 1 (PFDN1), cell division cycle 25B (CDC25B), and myosin light chain kinase 3 (MYLK3).

The methods herein can further include comparing the expression of colon cancer-related molecules and/or methylation of colon cancer-related molecules (such as COAD-related molecules) from colon cancer-related pathways (such as COAD-related pathways) measured in a sample obtained from a subject, for example, a COAD sample or stool sample. In some examples, the measured expression and/or methylation of colon cancer-related molecules (COAD-related molecules) from colon cancer-related pathways (such as COAD-related pathways) is compared with the expression and/or methylation of COAD-related molecules in a control representing expression for the COAD-related molecules expected in a colon cancer that does not positively respond to a chemotherapy (such as a subject with cancer that will not be treated by FOLFOX (such as a reduction in the size or metastasis of the tumor), and/or who does develop resistance to a chemotherapy such as FOLFOX) or does positively respond to a chemotherapy such as FOLFOX (such as a subject with cancer that will be treated by the a chemotherapy such as FOLFOX (such as a reduction in the size or metastasis of the tumor), and/or who does not develop resistance to a chemotherapy such as FOLFOX). Where differential expression and/or methylation is measured compared with a control representing expression and/or methylation for the COAD-related molecules expected in a colon cancer that does not positively respond to a chemotherapy such as FOLFOX, or similar expression and/or methylation is measured compared with a control representing expression and/or methylation expected in a colon cancer that does positively respond to a chemotherapy such as FOLFOX, the subject can be identified as a subject having a colon cancer that responds positively to a chemotherapy such as FOLFOX. Conversely, where similar expression and/or methylation is measured compared with a control representing expression and/or methylation for the COAD-related molecules expected in a colon cancer that does not positively respond to a chemotherapy such as FOLFOX, or differential expression and/or methylation is measured compared with a control representing expression and/or methylation expected in a colon cancer that does positively respond to a chemotherapy such as FOLFOX, the subject can be identified as a subject who has a colon cancer that does not respond positively to a chemotherapy such as FOLFOX. In some examples, the methods include administering a chemotherapy such as FOLFOX to a subject identified as one who responds positively to FOLFOX, thereby treating the colon cancer in the subject. In other examples, the methods include administering other types of COAD therapy (such as surgery, radiation therapy, targeted therapy, immunotherapy, or palliative care) to a subject identified as one who does not respond positively to a chemotherapy such as FOLFOX, thereby treating the subject.

Evaluating Expression and Methylation in Subjects with Colon Adenocarcinoma

In specific, non-limiting examples, the methods can be used to treat subjects with COAD or identify subjects with COAD that respond positively to chemotherapy (such as folinic acid, fluorouracil, and oxaliplatin, FOLFOX). In some examples, the methods herein can further include comparing the expression and/or methylation of COAD-related molecules (such as mRNA and DNA, respectively) from COAD-related pathways measured in a sample (such as a colon cancer sample or a stool/fecal sample) obtained from a subject. In some examples, the measured expression (such as expression of COAD-related molecules from elongation and processing of capped transcripts, processing of capped intron containing pre-mRNA, S phase, and calcium signaling pathways or SF3B3, PRPF6, CDC25B, and MYLK3) and/or methylation (such as methylation of COAD-related molecules from pre-mRNA, protein metabolism, S phase, and calcium signaling pathways or PFDN1, CDC25B, and MYLK3) of COAD-related molecules from COAD-related pathways is compared with the expression (such as expression of COAD-related molecules from elongation and processing of capped transcripts, processing of capped intron containing pre-mRNA, S phase, and calcium signaling pathways or SF3B3, PRPF6, CDC25B, and MYLK3) and/or methylation (such as methylation of COAD-related molecules from pre-mRNA, protein metabolism, S phase, and calcium signaling pathways or PFDN1, CDC25B, and MYLK3) of COAD-related molecules in a control representing expression for the COAD-related molecules expected in a COAD that does not positively respond to a FOLFOX or does positively respond to FOLFOX.

Where differential expression (such as expression of COAD-related molecules from elongation and processing of capped transcripts, processing of capped intron containing pre-mRNA, S phase, and calcium signaling pathways or SF3B3, PRPF6, CDC25B, and MYLK3) and/or methylation (such as methylation of COAD-related molecules from pre-mRNA, protein metabolism, S phase, and calcium signaling pathways or PFDN1, CDC25B, and MYLK3) is measured compared with a control representing expression and/or methylation for the COAD-related molecules expected in a COAD that does not positively respond to a chemotherapy (i.e., FOLFOX), or similar expression (such as expression of COAD-related molecules from elongation and processing of capped transcripts, processing of capped intron containing pre-mRNA, S phase, and calcium signaling pathways or SF3B3, PRPF6, CDC25B, and MYLK3) and/or methylation (such as methylation of COAD-related molecules from pre-mRNA, protein metabolism, S phase, and calcium signaling pathways or PFDN1, CDC25B, and MYLK3) is measured compared with a control representing expression and/or methylation expected in aa COAD that does positively respond to FOLFOX, the subject can be identified as a subject having a COAD that responds positively to FOLFOX.

Conversely, where similar expression (such as expression of COAD-related molecules from elongation and processing of capped transcripts, processing of capped intron containing pre-mRNA, S phase, and calcium signaling pathways or SF3B3, PRPF6, CDC25B, and MYLK3) and/or methylation (such as methylation of COAD-related molecules from pre-mRNA, protein metabolism, S phase, and calcium signaling pathways or PFDN1, CDC25B, and MYLK3) is measured compared with a control representing expression and/or methylation for the COAD-related molecules expected in a COAD that does not positively respond to a FOLFOX, or differential expression (such as expression of COAD-related molecules from elongation and processing of capped transcripts, processing of capped intron containing pre-mRNA, S phase, and calcium signaling pathways or SF3B3, PRPF6, CDC25B, and MYLK3) and/or methylation (such as methylation of COAD-related molecules from pre-mRNA, protein metabolism, S phase, and calcium signaling pathways or PFDN1, CDC25B, and MYLK3) is measured compared with a control representing expression and/or methylation expected in a COAD that does positively respond to FOLFOX, the subject can be identified as a subject having a COAD that does not respond positively to FOLFOX.

In some examples, the methods include administering chemotherapy (such as folinic acid, fluorouracil, and oxaliplatin) to a subject identified as one who responds positively to chemotherapy, thereby treating the subject. In other examples, the methods include administering other types of COAD therapy (such as surgery, radiation therapy, targeted therapy, immunotherapy, or palliative care) to a subject identified as one who does not respond positively to chemotherapy, thereby treating the subject.

Detecting Expression and/or Methylation

As described herein, expression of any cancer-related molecule or combinations thereof disclosed herein (such as cancer-related molecules from cancer-related pathways that include chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways, such as CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3) can be detected alone or in combination using a variety of methods. Expression of nucleic acid molecules (e.g., mRNA, cDNA) or protein is contemplated herein. Exemplary nucleic acid sequences that can be detected are provided in the sequence listing. One skilled in the art can use these sequences to identify the corresponding mRNA and protein sequence encoded thereby, which can also be detected.

Further, DNA methylation of any cancer-related molecules or combination thereof disclosed herein (such as cancer-related molecules from cancer-related pathways, including chemokine receptor, mitotic cell cycle, immune network for IgA production, mRNA splicing, protein metabolism, RNA degradation, ribosome pathway, cytokine-cytokine receptor interaction, neuroactive ligand-receptor interaction, SLC-mediated transmembrane transport, translation, processing of capped intron containing pre-mRNA, protein metabolism, S phase, and calcium signaling pathways or CCL22, CCR9, POLR2C, FGFR1OP, CCT4, LSM7, RPLP2, CCL11, GABRA1, SLC44A4, RPL14, PFDN1, CDC25B, or MYLK3) can also be detected alone or in combination using a variety of methods.

1. Methods for detecting mRNA Expression

Gene expression can be evaluated by detecting mRNA encoding the gene of interest. Thus, the disclosed methods can include evaluating mRNA encoding cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways or CCL22, CCR9, POLR2C, FGFR1OP, CCT4, PDE7A, DTYMK, ARPC1A, RPLP2, CCL11, ERCC1, U2AF1, SF3B3, PRPF6, CDC25B, or MYLK3. In some examples, mRNA expression is quantified.

RNA can be isolated from a cancer sample (such as lung or colorectal cancer) or other sample (e.g., blood, sputum, or stool sample) from a subject, for example using commercially available kits, such as those from QIAGEN®. General methods for mRNA extraction are disclosed in, for example, including Ausubel et al., Current Protocols of Molecular Biology, John Wiley and Sons (1997). RNA can be extracted from paraffin embedded tissues (e.g., see Rupp and Locker, Lab Invest. 56:A67 (1987), and De Andres et al., BioTechniques 18:42044 (1995)). Total RNA from cells in culture (such as those obtained from a subject) can be isolated using QIAGIN® RNeasy mini-columns. Other commercially available RNA isolation kits include MASTERPURE®. Complete DNA and RNA Purification Kit (EPICENTRE® Madison, Wis.), and Paraffin Block RNA Isolation Kit (Ambion, Inc.). Total RNA from tissue samples can be isolated using RNA Stat-60 (Tel-Test). RNA prepared from tumor or other biological sample can be isolated, for example, by cesium chloride density gradient centrifugation.

Methods of gene expression profiling include methods based on hybridization analysis of polynucleotides, methods based on sequencing of polynucleotides, and proteomics-based methods. In some examples, mRNA expression in a sample is quantified using northern blotting or in situ hybridization (Parker & Barnes, Methods in Molecular Biology 106:247-283, 1999); RNAse protection assays (Hod, Biotechniques 13:852-4, 1992); and PCR-based methods, such as reverse transcription polymerase chain reaction (RT-PCR) (Weis et al., Trends in Genetics 8:263-4, 1992). Alternatively, antibodies can be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

In one example, RT-PCR can be used. Generally, the first step in gene expression profiling by RT-PCR is the reverse transcription of the RNA template into cDNA, followed by its exponential amplification in a PCR reaction. Two commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase. TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

To minimize errors and the effect of sample-to-sample variation, RT-PCR can be performed using an internal standard. The ideal internal standard is expressed at a constant level among different tissues, and is unaffected by the experimental treatment. RNAs commonly used to normalize patterns of gene expression are mRNAs for the housekeeping genes glyceraldehyde-3-phosphate-dehydrogenase (GAPDH), beta-actin, tubulin, and 18S ribosomal RNA.

A variation of RT-PCR is real time quantitative RT-PCR, which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Held et al., Genome Research 6:986 994, 1996). Quantitative PCR is also described in U.S. Pat. No. 5,538,848. Related probes and quantitative amplification procedures are described in U.S. Pat. Nos. 5,716,784 and 5,723,591. Instruments for carrying out quantitative PCR in microtiter plates are available from PE Applied Biosystems, 850 Lincoln Centre Drive, Foster City, Calif. 94404 under the trademark ABI PRISM® 7700.

The steps of a representative protocol for quantifying gene expression using fixed, paraffin-embedded tissues as the RNA source, including mRNA isolation, purification, primer extension and amplification are given in various publications (see Godfrey et al., J. Mol. Diag. 2:84 91, 2000; Specht et al., Am. J. Pathol. 158:419-29, 2001). Briefly, a representative process starts with cutting about 10 μm thick sections of paraffin-embedded tumor tissue samples or adjacent non-cancerous tissue. The RNA is then extracted, and protein and DNA are removed. Alternatively, RNA is located directly from a tumor sample or other tissue sample. After analysis of the RNA concentration, RNA repair and/or amplification steps can be included, if necessary, and RNA is reverse transcribed using gene specific promoters followed by RT-PCR. The primers used for the amplification are selected so as to amplify a unique segment of the gene of interest, such as mRNA encoding cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways or CCL22, CCR9, POLR2C, FGFR1OP, CCT4, PDE7A, DTYMK, ARPC1A, RPLP2, CCL11, ERCC1, U2AF1, SF3B3, PRPF6, CDC25B, or MYLK3. In some embodiments, expression of other genes is also detected. Primers that can be used to amplify cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways or CCL22, CCR9, POLR2C, FGFR1OP, CCT4, PDE7A, DTYMK, ARPC1A, RPLP2, CCL11, ERCC1, U2AF1, SF3B3, PRPF6, CDC25B, or MYLK3 are commercially available or can be designed and synthesized (such as based on SEQ ID NOS: 1-14 and 20-21). In some examples, the primers specifically hybridize to a promoter or promoter region of a cancer-related molecule from a cancer-related pathway, such as chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways or CCL22, CCR9, POLR2C, FGFR1OP, CCT4, PDE7A, DTYMK, ARPC1A, RPLP2, CCL11, ERCC1, U2AF1, SF3B3, PRPF6, CDC25B, or MYLK3.

An alternative quantitative nucleic acid amplification procedure is described in U.S. Pat. No. 5,219,727. In this procedure, the amount of a target sequence in a sample is determined by simultaneously amplifying the target sequence and an internal standard nucleic acid segment. The amount of amplified DNA from each segment is determined and compared to a standard curve to determine the amount of the target nucleic acid segment that was present in the sample prior to amplification.

In some embodiments of this method, the expression of a “housekeeping” gene or “internal control” can also be evaluated. These terms include any constitutively or globally expressed gene whose presence enables an assessment of mRNA levels provided herein. Such an assessment includes a determination of the overall constitutive level of gene transcription and a control for variations in RNA recovery. Exemplary housekeeping genes include b-actin and tubulin.

In some examples, gene expression is identified or confirmed using a microarray technique. Thus, the expression profile can be measured in either fresh or paraffin-embedded tumor tissue, using microarray technology. In this method, nucleic acid sequences (including cDNAs and oligonucleotides) encoding cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways or CCL22, CCR9, POLR2C, FGFR1OP, CCT4, PDE7A, DTYMK, ARPC1A, RPLP2, CCL11, ERCC1, U2AF1, SF3B3, PRPF6, CDC25B, or MYLK3 are plated, or arrayed, on a microchip substrate. The arrayed sequences are then hybridized with specific DNA probes from cells or tissues of interest. Just as in the RT-PCR method, the source of mRNA typically is total RNA isolated from human tumors, and optionally from corresponding noncancerous tissue and normal tissues or cell lines.

In a specific embodiment of the microarray technique, PCR amplified inserts of cDNA clones are applied to a substrate in a dense array. At least probes specific for nucleotide sequences encoding cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways or CCL22, CCR9, POLR2C, FGFR1OP, CCT4, PDE7A, DTYMK, ARPC1A, RPLP2, CCL11, ERCC1, U2AF1, SF3B3, PRPF6, CDC25B, or MYLK3 (and, in some examples, one or more housekeeping genes) are applied to the substrate, and the array can consist essentially of, or consist of these sequences. The microarrayed nucleic acids are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways or CCL22, CCR9, POLR2C, FGFR1OP, CCT4, PDE7A, DTYMK, ARPC1A, RPLP2, CCL11, ERCC1, U2AF1, SF3B3, PRPF6, CDC25B, or MYLK3. Microarray analysis can be performed by commercially available equipment, following manufacturer's protocols.

Serial analysis of gene expression (SAGE) allows the simultaneous and quantitative analysis of a large number of gene transcripts, without the need of providing an individual hybridization probe for each transcript. First, a short sequence tag (about 10-14 base pairs) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag (see, for example, Velculescu et al., Science 270:484-7, 1995; and Velculescu et al., Cell 88:243-51, 1997, herein incorporated by reference in their entireties).

In situ hybridization (ISH) is another method for detecting and comparing expression of cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways or CCL22, CCR9, POLR2C, FGFR1OP, CCT4, PDE7A, DTYMK, ARPC1A, RPLP2, CCL11, ERCC1, U2AF1, SF3B3, PRPF6, CDC25B, or MYLK3. ISH applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH can be used to assay expression patterns in a tissue, such as the expression of cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways or CCL22, CCR9, POLR2C, FGFR1OP, CCT4, PDE7A, DTYMK, ARPC1A, RPLP2, CCL11, ERCC1, U2AF1, SF3B3, PRPF6, CDC25B, or MYLK3.

Sample cells or tissues can be treated to increase their permeability to allow a probe to enter the cells, such as a gene-specific probe for cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways or CCL22, CCR9, POLR2C, FGFR1OP, CCT4, PDE7A, DTYMK, ARPC1A, RPLP2, CCL11, ERCC1, U2AF1, SF3B3, PRPF6, CDC25B, or MYLK3. The probe is added to the treated cells, allowed to hybridize at pertinent temperature, and excess probe is washed away. The probe can be labeled, for example with a radioactive, fluorescent or antigenic tag, so that the probe's location and quantity in the tissue can be determined, for example using autoradiography, fluorescence microscopy or immunoassay. Probes can be designed such that the probes specifically bind a gene of interest because cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways or CCL22, CCR9, POLR2C, FGFR1OP, CCT4, PDE7A, DTYMK, ARPC1A, RPLP2, CCL11, ERCC1, U2AF1, SF3B3, PRPF6, CDC25B, or MYLK3 are known.

In situ PCR is the PCR-based amplification of the target nucleic acid sequences prior to ISH. For detection of RNA, an intracellular reverse transcription step is introduced to generate complementary DNA from RNA templates prior to in situ PCR. This enables detection of low copy RNA sequences.

Prior to in situ PCR, cells or tissue samples can be fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR, the cells are cytocentrifuged onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens.

Detection of intracellular PCR products can be achieved by ISH with PCR-product specific probes, or direct in situ PCR without ISH through direct detection of labeled nucleotides (such as digoxigenin-11-dUTP, fluorescein-dUTP, 3H-CTP or biotin-16-dUTP), which have been incorporated into the PCR products during thermal cycling.

Gene expression can also be detected and quantitated using the nCounter® technology developed by NanoString (Seattle, Wash.; see, for example, U.S. Pat. Nos. 7,473,767; 7,919,237; and 9,371,563, which are herein incorporated by reference in their entireties). The nCounter® analysis system utilizes a digital color-coded barcode technology that is based on direct multiplexed measurement of gene expression. The technology uses molecular “barcodes” and single molecule imaging to detect and count hundreds of unique transcripts in a single reaction. Each color-coded barcode is attached to a single target-specific probe corresponding to a gene of interest (such as a TACE-response gene). Mixed together with controls, they form a multiplexed CodeSet.

Each color-coded barcode represents a single target molecule. Barcodes hybridize directly to target molecules and can be individually counted without the need for amplification. The method includes three steps: (1) hybridization; (2) purification and immobilization; and (3) counting. The technology employs two approximately 50 base probes per mRNA that hybridize in solution. The reporter probe carries the signal; the capture probe allows the complex to be immobilized for data collection. After hybridization, the excess probes are removed and the probe/target complexes are aligned and immobilized in the nCounter® cartridge. Sample cartridges are placed in the digital analyzer for data collection. Color codes on the surface of the cartridge are counted and tabulated for each target molecule. This method is described in, for example, U.S. Pat. No. 7,919,237; and U.S. Patent Application Publication Nos. 20100015607; 20100112710; 20130017971, which are herein incorporated by reference in their entireties. Information on this technology can also be found on the company's website (nanostring.com).

2. Arrays for Profiling Gene Expression

In particular embodiments, arrays (such as a solid support) are used to evaluate gene expression, for example to determine if a patient with cancer (such as lung or colorectal cancer) will respond to chemotherapy. Such arrays can include a set of specific binding agents (such as nucleic acid probes and/or primers specific for cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways or CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3. When describing an array that consists essentially of probes or primers specific for cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways, such as CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3, such an array includes probes or primers specific for the gene or genes, and can further include control probes or primers, such as 1-10 control probes or primers (for example to confirm the incubation conditions are sufficient). In some examples, the array may further comprise additional, such as 1, 2, 3, 4, or 5 additional probes for other genes. In some examples, the array includes 1-10 housekeeping-specific probes or primers. In one example, an array is a multi-well plate (e.g., 98 or 364 well plate).

In one example, the array includes, consists essentially of, or consists of probes or primers (such as an oligonucleotide or antibody) that can recognize cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways or CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3 (and, in some examples, also 1-10 housekeeping genes). The oligonucleotide probes or primers can further include one or more detectable labels, to permit detection of hybridization signals between the probe and target sequence (such as cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways or CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3).

a. Array Substrates

The solid support of the array can be formed from an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, etyleneacrylic acid, thylene methacrylic acid, and blends of copolymers thereof (see U.S. Pat. No. 5,985,567).

In one example, the solid support surface is polypropylene. In another example, a surface activated organic polymer is used as the solid support surface. One example of a surface activated organic polymer is a polypropylene material aminated via radio frequency plasma discharge. Such materials are easily utilized for the attachment of nucleotide molecules. The amine groups on the activated organic polymers are reactive with nucleotide molecules such that the nucleotide molecules can be bound to the polymers. Other reactive groups can also be used, such as carboxylated, hydroxylated, thiolated, or active ester groups.

b. Array Formats

A wide variety of array formats can be employed. One example includes a linear array of oligonucleotide bands, generally referred to in the art as a dipstick. Another suitable format includes a two-dimensional pattern of discrete cells (such as 4096 squares in a 64 by 64 array). Other array formats including, but not limited to slot (rectangular) and circular arrays are equally suitable for use. In some examples, the array is a multi-well plate. In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. The array can include biaxially oriented polypropylene (BOPP) films, which in addition to their durability, exhibit a low background fluorescence.

The array formats can be included in a variety of different types of formats. A “format” includes any format to which probes, primers or antibodies can be affixed, such as microtiter plates (e.g., multi-well plates), test tubes, inorganic sheets, dipsticks, and the like. For example, when the solid support is a polypropylene thread, one or more polypropylene threads can be affixed to a plastic dipstick-type device; polypropylene membranes can be affixed to glass slides.

The arrays of can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are describe in Matson et al., Anal. Biochem. 217:306-10, 1994. In one example, the oligonucleotides are synthesized onto the support using chemical techniques for preparing oligonucleotides on solid supports (such as see PCT applications WO 85/01051 and WO 89/10977, or U.S. Pat. No. 5,554,501).

The oligonucleotides can be bound to the polypropylene support by either the 3′ end of the oligonucleotide or by the 5′ end of the oligonucleotide. In one example, the oligonucleotides are bound to the solid support by the 3′ end. In general, the internal complementarity of an oligonucleotide probe in the region of the 3′ end and the 5′ end determines binding to the support.

In particular examples, the oligonucleotide probes on the array include one or more labels, that permit detection of oligonucleotide probe:target sequence hybridization complexes.

3. Detecting Protein Expression

In some examples, expression of cancer-related proteins from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways, such as CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3 proteins is analyzed. Suitable biological samples include samples containing protein obtained from a cancer (such as a lung or colorectal cancer) or other sample (e.g., blood, feces, sputum) of a subject. An alteration in the amount of cancer-related proteins from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways, such as CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3 proteins in a tumor (such as a lung or colon tumor) from the subject relative to a control, such as an increase or decrease in protein expression, indicates whether the cancer (such as lung or colon cancer) will respond to chemotherapy, as described herein.

Antibodies specific for cancer-related proteins from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways, such as CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3 proteins can be used for protein detection and quantification, for example using an immunoassay method, such as those presented in Harlow and Lane (Antibodies, A Laboratory Manual, CSHL, New York, 1988).

Exemplary immunoassay formats include ELISA, Western blot, and RIA assays. Thus, protein levels of cancer-related proteins from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways, such as CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3 proteins in a cancer sample (such as a lung or colon cancer sample) can be evaluated using these methods. Immunohistochemical techniques can also be utilized protein detection and quantification. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998).

To quantify proteins, a biological sample of a subject that includes cellular proteins can be used. Quantification of cancer-related proteins from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways, such as CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3 proteins can be achieved by immunoassay methods. The amount cancer-related protein from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways, such as CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3 protein can be assessed in a cancer sample (such as a lung or colon cancer sample) and optionally in cancer samples (such as lung or colon cancer samples) from patients known to respond to chemotherapy (or to not respond). The amounts of cancer-related protein from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways, such as CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3 protein in the tumor can be compared to levels of the protein found in cancer samples (such as lung or colon cancer) from patients known to respond to chemotherapy (or not respond) or other control (such as a standard value or reference value). A significant increase or decrease in the amount can be evaluated using statistical methods.

Quantitative spectroscopic approaches, such as SELDI, can be used to analyze expression of cancer-related proteins from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways, such as CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3 expression in a sample (such as a lung or colon cancer sample). In one example, surface-enhanced laser desorption-ionization time-of-flight (SELDI-TOF) mass spectrometry is used to detect protein expression, for example by using the ProteinChip™ (Ciphergen Biosystems, Palo Alto, Calif.). Such methods are well known in the art (for example see U.S. Pat. Nos. 5,719,060; 6,897,072; and 6,881,586). SELDI is a solid phase method for desorption in which the analyte is presented to the energy stream on a surface that enhances analyte capture or desorption.

The surface chemistry allows the bound analytes to be retained and unbound materials to be washed away. Subsequently, analytes bound to the surface (such as tumor-associated proteins) can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector. A plurality of detection means can be implemented in series to fully interrogate the analyte components and function associated with retained molecules at each location in the array.

Therefore, in a particular example, the chromatographic surface includes antibodies that specifically bind cancer-related proteins from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways, such as CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3. In other examples, the chromatographic surface consists essentially of, or consists of, antibodies that specifically bind cancer-related proteins from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, and calcium signaling pathways, such as CCL22, CCR9, POLR2C, FGFR1OP, CCT4, PDE7A, DTYMK, ARPC1A, RPLP2, CCL11, ERCC1, U2AF1, SF3B3, PRPF6, CDC25B, and/or MYLK3. In some examples, the chromatographic surface includes antibodies that bind other molecules, such as housekeeping proteins (e.g., tubulin, b-actin).

In another example, antibodies are immobilized onto the surface using a bacterial Fc binding support. The chromatographic surface is incubated with a sample, such as a sample of a lung or colon tumor. The antigens present in the sample can recognize the antibodies on the chromatographic surface. The unbound proteins and mass spectrometric interfering compounds are washed away and the proteins that are retained on the chromatographic surface are analyzed and detected by SELDI-TOF. The MS profile from the sample can be then compared using differential protein expression mapping, whereby relative expression levels of proteins at specific molecular weights are compared by a variety of statistical techniques and bioinformatic software systems.

4. Detecting DNA Methylation

DNA methylation can be determined for DNA encoding each of cancer-related molecules from cancer-related pathways, including chemokine receptor, mitotic cell cycle, immune network for IgA production, mRNA splicing, protein metabolism, RNA degradation, ribosome pathway, cytokine-cytokine receptor interaction, neuroactive ligand-receptor interaction, SLC-mediated transmembrane transport, translation, processing of capped intron containing pre-mRNA, protein metabolism, S phase, and calcium signaling pathways, such as CCL22, CCR9, POLR2C, FGFR1OP, CCT4, LSM7, RPLP2, CCL11, GABRA1, SLC44A4, RPL14, PFDN1, CDC25B, and/or MYLK3 in a cancer sample (such as a lung or colon cancer sample) or other sample (e.g., blood, sputum, or stool), and, in some examples, also a control sample (e.g., cancer samples, such as lung or colon cancer samples, from patients known to respond to chemotherapy (or to not respond)). Exemplary methods of detecting DNA methylation in a sample include bisulfite sequencing or conversion, pyrosequencing, HPLC-UV, LC-MS/MS, ELISA-based methods, and array or bead hybridization. In one example, the VeraCode Methylation technology from Illumina is used. For a review of such methods see Kurdyukov and Bullock (Biology 5:3, 2016). Thus, in some examples, cancer samples, for example, lung or colorectal cancer samples (or DNA isolated from such samples) are contacted with bisulfate, and can also be subjected to amplification and sequencing.

### B. Cancer Samples

The methods provided herein include detecting expression (e.g., mRNA expression) and/or DNA methylation of cancer-related molecules from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, calcium signaling, RNA degradation, neuroactive ligand-receptor interaction, SLC-mediated transmembrane transport, and translation pathways, such as CCL22, CCR9, POLR2C, FGFR1OP, CCT4, PDE7A, DTYMK, ARPC1A, RPLP2, CCL11, ERCC1, U2AF1, SF3B3, PRPF6, CDC25B, MYLK3, LSM7, GABRA1, SLC44A4, RPL14, and/or PFDN1 in cancer samples (such as lung or colon cancer samples, such as LUAD, LUSC, or COAD).

In some embodiments, the cancer samples (such as lung or colon cancer samples, such as LUAD, LUSC, or COAD) are obtained from subjects diagnosed with cancer (such as lung or colon cancer). A “sample” refers to part of a tissue that is either the entire tissue, or a diseased or healthy portion of the tissue. As described herein, cancer samples (such as lung or colon cancer samples) can be compared to a control. In some embodiments, the control is a cancer sample (such as a lung or colon cancer sample) obtained from a subject or group of subjects known to have favorably responded to chemotherapy (or not to have responded).

In other embodiments, the control is a standard or reference value based on an average of historical values. In some examples, the reference values are an average expression (e.g., mRNA expression) or DNA methylation value for each of a cancer-related molecule from cancer-related pathways, including chemokine receptor, mitotic cell cycle, immune network for IgA production, mRNA splicing, protein metabolism, G alpha signaling, nucleotide metabolism, actin Y, ribosome, cytokine-cytokine receptor interaction, DNA repair, transport of mature mRNA derived from an intron-containing transcript, elongation and processing of capped transcripts, processing of capped intron containing pre-mRNA, S phase, calcium signaling, RNA degradation, neuroactive ligand-receptor interaction, SLC-mediated transmembrane transport, and translation pathways, such as CCL22, CCR9, POLR2C, FGFR1OP, CCT4, PDE7A, DTYMK, ARPC1A, RPLP2, CCL11, ERCC1, U2AF1, SF3B3, PRPF6, CDC25B, MYLK3, LSM7, GABRA1, SLC44A4, RPL14, and/or PFDN1 in a cancer sample (such as a lung or colon cancer sample) obtained from a subject or group of subjects known to have favorably responded to chemotherapy (or not to have responded).

Tissue samples can be obtained from a subject, for example, from cancer patients (such as lung or colon cancer patients) who have undergone tumor resection as a form of treatment. In some embodiments, cancer samples (such as lung or colon cancer samples) are obtained by biopsy. Biopsy samples can be fresh, frozen or fixed, such as formalin-fixed and paraffin embedded. Samples can be removed from a patient surgically, by extraction (for example by hypodermic or other types of needles), by microdissection, by laser capture, or by other means.

In some examples, proteins and/or nucleic acid molecules (e.g., DNA, RNA, mRNA, and cDNA) are isolated or purified from the cancer sample (such as a lung or colon cancer sample). In some examples, the cancer sample (such as a lung or colon cancer sample) is used directly, or is concentrated, filtered, or diluted.

### EXAMPLES

Disclosed herein are systems and methods to uncover interplay between genomic and epigenomic mechanisms and elucidate the complexity of the chemotherapy response in cancer patients. These systems and methods integrate genomic information (such as mRNA expression) and epigenomic information (such as DNA methylation) from patient profiles to identify molecular pathways with significant alterations on genomic and epigenomic levels to distinguish favorable from poor chemotherapy treatment responses.

The systems and methods disclosed herein were used on patients with lung adenocarcinoma who received a carboplatin and paclitaxel combination chemotherapy (carboplatin-paclitaxel), a standard-of-care for treating advanced lung cancer. This integrative approach identified seven molecular pathways with significant epigenomic alterations that distinguish favorable from poor carboplatin-paclitaxel response, including chemokine receptors, mRNA splicing, G alpha signaling events, and immune network for IgA production. These pathways can be used to classify patients based on their risk of developing carboplatin-paclitaxel resistance in an independent patient cohort (log-rank p-value=0.0081), and their predictive ability is independent of and not affected by (i) signatures of overall lung cancer aggressiveness or (ii) commonly utilized covariates, such as age, gender, and stage at diagnosis (adjusted hazard ratio=14.0). Demonstrating the generalizability of these systems and methods, they were applied across additional chemotherapy regimens (i.e., cisplatin-vinorelbine, oxaliplatin-fluorouracil) and cancer types (i.e., lung squamous cell carcinoma and colorectal adenocarcinoma), showing their ability to accurately predict treatment response.

Thus, the systems and methods herein can be utilized to identify epigenomically altered pathways implicated in primary chemoresponse and effectively classify patients who would benefit from specific chemotherapy regimens or are at risk of resistance, significantly improving personalized therapeutic strategies and informed clinical decision making.

### Example 1—Methods

Lung adenocarcinoma patient cohorts: LUAD patient cohorts were obtained from publicly available data sources, including The Cancer Genome Atlas-Lung Adenocarcinoma (TCGA-LUAD) (Cancer Genome Atlas Research Network, Nature. 2014; 511(7511):543-50), Tang et al. (GSE42127) (Tang et al., Clin. Can. Res. 2013; 19(6):1577-86), Der et al. (GSE50081) (Der et al., J. Thor. Oncol. 2014; 9(1):59-64), and Zhu et al. (GSE14814) (Zhu et al., J. Clin. Oncol. 2010; 28(29):4417-24) datasets (Tables 2-5). The primary LUAD patient cohort that was utilized for reconstruction of epigenomic signatures of chemoresistance was obtained from The Cancer Genome Atlas (TCGA-LUAD) project (Cancer Genome Atlas Research Network, Nature. 2014; 511(7511):543-50) and downloaded from the Genomics Data Commons database (GDC; portal.gdc.cancer.gov) on February 2017. Clinical information (such as clinical files, follow-ups, and treatment data) for these datasets were obtained from the TCGA GDC legacy archive (portal.gdc.cancer.gov).

TABLE 2
Clinical and pathological features of lung
adenocarcinoma (LUAD) patient cohorts
treated with carboplatin-paclitaxel, used
for signature, validation, and negative controls.

| | Signature | Validation | Negative controls | |
|---|---|---|---|---|
| Description | TCGA | Tang et al. | Tang et al. | Der et al. |
| | | (treated) | (not treated) | |
| Accession # | TCGA- | GSE42127** | GSE42127** | GSE50081 |
| | LUAD* | | | *** |
| Patients | 14 | 39 | 94 | 127 |
| Sample | surgery | surgery | surgery | surgery |
| collection | | | | |
| Histological | | | | |
| subtype | | | | |
| mixed | 1 | | | |
| acinar | 1 | NA | NA | NA |
| papillary | | | | |
| mucinous | | | | |
| lepidic | | | | |
| solid | | | | |
| NOS | 12 | | | |
| Anatomic Site | | | | |
| Left-Upper | 5 | NA | NA | NA |
| Left-Lower | 2 | | | |
| Right-Lower | 1 | | | |
| Right-Middle | 2 | | | |
| Right-Upper | 4 | | | |
| Gender | | | | |
| Female | 9 | 16 | 49 | 62 |
| Male | 5 | 23 | 45 | 65 |
| Tumor Stage | | | | |
| (Pathological) | | | | |
| I | | | | |
| IA | | 1 | 31 | 36 |
| IB | 1 | 21 | 36 | 56 |
| II | | | | |
| IIA | 1 | 1 | 5 | 7 |
| IIB | 4 | 5 | 11 | 28 |
| IIIA | 4 | 3 | 4 | |
| IIIB | 1 | 8 | 5 | |
| IV | 1 | | 1 | |
| NA | 2 | | 1 | |
| Smoking Status | | | | |
| 1 | 2 | | | |
| 2 | 4 | | | |
| 3 | 3 | NA | NA | NA |
| 4 | 5 | | | |
| 5 | | | | |
| 6 | | | | |

Notes:
NA = Not available,
NOS = Not otherwise specified.
Smoking status: 1 = lifelong non-smoker (<100 cigarettes smoked in lifetime),
2 = current smoker (includes daily smokers and non-daily smokers (or occasional smokers),
3 = current reformed smoker for > 15 years,
4 = current reformed smoker for ≤ 15 years,
5 = current reformed smoker, duration not specified, and
6 = smoking history not documented.
*Comprehensive molecular profiling of lung adenocarcinoma. Nature. 2014; 511(7511): 543-50
**Tang et al., Clinical Can. Res. 2013; 19(6): 1577-86..
***Der et al., Journal of thoratic oncology, 2014; 9(1): 59-64.

TABLE 3
Clinical and pathological features of lung adenocarcinoma (LUAD)
patient cohorts treated with cisplatin-vinorelbine,
used for signature and validation.

| | Signature | Validation |
|---|---|---|
| Description | TCGA | Zhu et al. |
| Accession # | TCGA-LUAD* | GSE14814** |
| Patients | 8 | 39 |
| Sample collection | surgery | surgery |
| Histological subtype | | |
| mixed | 6 | |
| acinar | 1 | 9 |
| papillary | | 5 |
| mucinous | | 1 |
| lepidic | | 1 |
| solid | | 9 |
| NOS | 1 | 14 |
| Anatomic Site | | |
| Left-Upper | 2 | |
| Left-Lower | | NA |
| Right-Lower | 2 | |
| Right-Middle | 1 | |
| Right-Upper | 3 | |
| Gender | | |
| Female | 5 | 20 |
| Male | 3 | 19 |
| Tumor Stage | | |
| (Pathological) | | |
| I | | |
| IA | | 8 |
| IB | 1 | 14 |
| II | | |
| IIA | 3 | 11 |
| IIB | 1 | 6 |
| IIIA | 2 | |
| IIIB | | |
| IV | 1 | |
| NA | | |
| Smoking Status | | |
| 1 | 1 | |
| 2 | | |
| 3 | 4 | NA |
| 4 | 3 | |
| 5 | | |
| 6 | | |

Notes:
NA = Not available,
NOS = Not otherwise specified.
Smoking status: 1 = lifelong non-smoker (<100 cigarettes smoked in lifetime),
2 = current smoker (includes daily smokers and non-daily smokers (or occasional smokers),
3 = current reformed smoker for > 15 years,
4 = current reformed smoker for ≤ 15 years,
5 = current reformed smoker, duration not specified, and
6 = smoking history not documented.
*Comprehensive molecular profiling of lung adenocarcinoma. Nature. 2014; 511(7511): 543-50
**Zhu et al., Journal of clinical oncology, 2010; 28(29): 4417-24.

TABLE 4
Clinical and pathological features of lung
squamous cell carcinoma (LUSC) patient
cohorts treated with cisplatin-vinorelbine,
used for signature and validation.

| | Signature | Validation |
|---|---|---|
| Description | TCGA | Zhu et al. |
| Accession # | TCGA-LUSC* | GSE14814** |
| Patients | 8 | 26 |
| Sample collection | surgery | surgery |
| Histological subtype | | |
| mixed | | |
| acinar | | |
| papillary | | |
| mucinous | | |
| lepidic | | |
| solid | | |
| NOS | 8 | 26 |
| Anatomic Site | | |
| Left-Upper | 2 | |
| Left-Lower | | NA |
| Right-Lower | 4 | |
| Right-Middle | 1 | |
| Right-Upper | 1 | |
| Gender | | |
| Female | 1 | 3 |
| Male | 7 | 23 |
| Tumor Stage | | |
| (Pathological) | | |
| I | | 13 |
| IA | | |
| IB | 2 | |
| II | | 13 |
| IIA | 1 | |
| IIB | 4 | |
| IIIA | 1 | |
| IIIB | | |
| IV | | |
| NA | | |
| Smoking Status | | |
| 1 | | |
| 2 | | |
| 3 | 2 | NA |
| 4 | 6 | |
| 5 | | |
| 6 | | |

Notes:
NA = Not available,
NOS = Not otherwise specified.
Smoking status: 1 = lifelong non-smoker (<100 cigarettes smoked in lifetime),
2 = current smoker (includes daily smokers and non-daily smokers (or occasional smokers),
3 = current reformed smoker for >15 years,
4 = current reformed smoker for ≤ 15 years,
5 = current reformed smoker, duration not specified, and
6 = smoking history not documented.
*Nature. 2012; 489(7417): 519-25
**Zhu et al., J. Clin. Oncol., 2010; 28(29): 4417-2

TABLE 5
Clinical and pathological features of colorectal adenocarcinoma
(COAD) patient cohorts treated with FOLFOX
(folinic acid, fluorouracil, oxaliplatin),
used for signature and validation.

| | Signature | Validation |
|---|---|---|
| Description | TCGA | Marisa et al. |
| Accession # | TCGA-COAD* | GSE39582** |
| Patients | 8 | 23 |
| Sample collection | surgery | surgery |
| Histological subtype | | |
| Ascending Colon | 1 | |
| Cecum | 2 | NA |
| Descending Colon | 1 | |
| Sigmoid Colon | 3 | |
| NA | 1 | |
| Gender | | |
| Female | 4 | 8 |
| Male | 4 | 15 |
| Tumor Stage | | |
| (Pathological) | | |
| I | | |
| IA | | |
| IB | | |
| II | | |
| IIA | 1 | 2 |
| IIB | | 1 |
| III | 1 | |
| IIIA | 1 | 3 |
| IIIB | 4 | 3 |
| IIIC | 1 | 3 |
| IV | | 11 |

Notes:
NA = Not available.
*Nature. 2012; 487(7407): 330-7.
**Marisa et al., PLoS medicine. 2013; 10(5): e1001453

To study primary resistance to the carboplatin-paclitaxel combination (Table 2) in LUAD, the patients selected had primary tumors obtained at surgery (n=14) and did not receive neo-adjuvant treatment (no therapy prior to sample collection) but were treated with an adjuvant carboplatin (platinum-based alkylating chemotherapy) and paclitaxel (non-platinum based plant alkaloid chemotherapy taxane) combination. These patients were further monitored for disease progression; disease progression was defined as a new tumor event, including tumor re-occurrence, and local and distant metastases. TCGA-LUAD mRNA expression (RNA seq) data were profiled using an Illumina HiSeq 2000, and DNA methylation was profiled using an Illumina Infinium Human Methylation (HM450) array. For validation studies, the Tang et al. (Tang et al., Clin. Can. Res. 2013; 19(6):1577-86) (GSE42127) cohort was used, which captures primary LUAD tumors obtained at surgery (n=39) and that were not pre-treated (no neoadjuvant treatment) but treated with an adjuvant carboplatin and taxane (paclitaxel) chemotherapy combination and profiled on an Illumina HumanWG-6 v3.0 expression beadchip. Cohorts used for negative controls included (i) the Der et al. (Der et al., J. Thor. Oncol. 2014; 9(1):59-64) (GSE50081) patient cohort with LUAD that never received treatment (n=127), which was profiled using an Affymetrix Human Genome U133 Plus 2.0 Array; (ii) Tang et al. (Tang et al., Clin. Can. Res. 2013; 19(6):1577-86) (GSE42127) patient cohort with LUAD that did not receive any treatment (n=94), profiled on Illumina HumanWG-6 v3.0 expression beadchip (Table 2).

Signatures of LUAD aggressiveness were obtained from: (i) Larsen et al. (Larsen et al., Clin. Can. Res. 2007; 13(10):2946-54), which identified 54 prognostic LUAD markers; (ii) Beer et al. (Beer et al., Nature medicine. 2002; 8(8):816-24), which identified 50 prognostic LUAD markers; and (iii) Tang et al. (Tang et al., Clin. Can. Res. 2013; 19(6):1577-86), which identified 12 prognostic non-small cell lung cancer markers (non-small cell lung cancer is a class of lung cancer, which includes LUAD).

Gene expression and DNA methylation analysis: For the RNA-seq analysis, the variance for raw RNA-seq counts was normalized and stabilized using the DESeq2 (Love et al., Genome Biology. 2014; 15(12):550) R package. DNA methylation values for each site were reported as β (Beta) values, which were subsequently converted to M-values (Du et al., BMC Bioinformatics. 2010; 11(1):587) for statistical analysis, using the beta2m function in the Lumi (Du et al., Bioinformatics. 2008; 24(13):1547-8) R package. To avoid redundancy introduced by multiple sites present for each gene, one CpG site was selected per gene through the coefficient of variation analysis, where a site with the highest coefficient of variation was selected for each gene.

Defining signatures of chemotherapy response: The next step was to define a signature of response to carboplatin-paclitaxel combination. For this, clinical data was analyzed from 14 patients that received carboplatin-paclitaxel chemo treatment in the TCGA-LUAD patient cohort (Table 2). To identify patients that failed the treatment and patients with a favorable response, the time between carboplatin-paclitaxel start and disease progression (a new tumor event was defined as tumor reappearance or local or distant metastases) or latest follow-up was analyzed for each patient. Next, a failed/poor treatment response was defined as patients whose disease progressed within 1 year of treatment start and a favorable response was defined as patients who stayed disease progression-free for over 2 years. To ensure that patients were not biased by initial tumor aggressiveness, local or distant metastatic burden, age, or smoking status, patients from each group were selected with similar distributions for (i) age, (ii) gender, (iii) tumor stage at diagnosis, and (iv) smoking status (Table 6), which defined feature-comparable groups of 4 poor-response and 4 favorable-response patients, utilized for further analysis.

TABLE 6
Clinical profiles of carboplatin-paclitaxel treated patients with poor (n = 4) and
favorable (n = 4) treatment response from the TCGA-LUAD cohort.

| | | Time to | | | | | | |
|---|---|---|---|---|---|---|---|---|
| | | event | | | | | | |
| | | or | | | | | | Observed |
| | | follow- | | | Tumor | | # | treatment |
| Treatment | Patient | up | | | stage at | Smoking | pack | related event |
| response | ID | (days) | Age | Gender | diagnosis | status | years | or follow-up |
| poor | 6712 | 116 | 71 | male | IIA | 4 | NA | new tumor |
| response | | | | | | | | event |
| | 5051 | 122 | 42 | female | IIIA | 4 | 30 | new tumor |
| | | | | | | | | event |
| | 6979 | 138 | 59 | female | IIB | 3 | NA | new tumor |
| | | | | | | | | event |
| | A4VP | 153 | 66 | female | IIIA | 4 | 20 | new tumor |
| | | | | | | | | event |
| favorable | 4666 | 744 | 52 | female | IV | 4 | 10 | no event, |
| response | | | | | | | | follow-up |
| | 5899 | 784 | 58 | male | IIA | 2 | NA | no event, |
| | | | | | | | | follow-up |
| | 1678 | 1120 | 70 | female | IIB | 3 | 20 | no event, |
| | | | | | | | | follow-up |
| | 1596 | 2031 | 55 | male | IIB | 2 | 50 | no event, |
| | | | | | | | | follow-up |

Notes:
NA = not available.
Smoking status: 1 = lifelong non-smoker (<100 cigarettes smoked in Lifetime),
2 = current smoker (includes daily smokers and non-daily smokers (or occasional smokers),
3 = current reformed smoker for > 15 years,
4 = current reformed smoker for ≤ 15 years,
5 = current reformed smoker, duration not specified, and
6 = smoking history not documented.

To determine the molecular characteristics that differ between poor response and favorable response, signatures of treatment response were defined at the genomic level (for example, differential expression) and epigenomic (for example, differential methylation) level between poor-response and favorable-response patient groups using the two-sample two-tailed Welch t-test (t.test function in R) (Welch, Biometrika. 1947; 34(1-2):28-35) in R studio version 3.3.2 (Team RC, Foundation for Statistical Computing; 2016. 2017), such that a differential expression signature was defined as a list of genes ranked on their differential expression (t-test values), and the differential methylation signature was defined as a list of genes based on the differential methylation of the corresponding site (t-test values).

Genomic and epigenomic pathway enrichment analysis: To identify molecular pathways significantly altered at the genomic and epigenomic levels (for example, FIG. 1), a pathway enrichment analysis was performed for a differential expression signature and differential methylation signature (for example, FIG. 7). For this analysis, the comprehensive C2 pathway database was used (Liberzon et al., Bioinformatics. 2011; 27(12):1739-40) (software.broadinstitute.org), which includes 833 pathways from the REACTOME (Fabregat et al., Nucleic Acids Res. 2016; 44(D1):D481-7), KEGG (Ogata et al., Nucleic Acids Res. 1999; 27(1):29-34), and BIOCARTA (Nishimura D., Biotech Software & Internet Report: The Computer Software Journal for Scient. 2001; 2(3):117-20) databases, and a pathway enrichment analysis was implemented using the Gene Set Enrichment Analysis (GSEA) (Subramanian et al., PNAS. 2005(102):15545-50), in which differential expression and differential methylation signatures were used as reference and collection of genes from each pathway was used as a query gene set. Normalized Enrichment Scores (NESs) and p-values were estimated using 1,000 gene permutations. This analysis estimated NESs for each of the 833 pathways, which reflects how much each pathway is enriched in the treatment response signature and defines a so-called pathway activity. A positive NES reflects pathway enrichment in the over-expressed part of the signature (a majority of pathway genes are over-expressed) and negative NES reflects pathway enrichment in the under-expressed part of the signature (a majority of pathway genes are under-expressed). Such pathway enrichment analysis is referred to as “signed” because it considers over- and under-expression of genes (with direction).

Further, to overcome limitations of such (signed) pathway enrichment analysis, which assumes that the pathway will be enriched only if majority of genes in the pathway are changed in the same direction (such as over-expressed or under-expressed, but not both), an “absolute valued” analysis was performed. For this, the pathway enrichment analysis was performed using the “absolute valued” differential expression signature, in which signature t-stat values was absolute valued to “collapse” positive and negative signature tails, as was previously performed in (Dutta et al., European Urology. 2017; 72(4):499-506). In this case, positive NESs reflect enrichment in a part of the signature with significant differential expression (including both over-expressed and under-expressed genes), and negative NESs reflect enrichment in the non-differentially expressed part of the signature (and are therefore not considered as significant). This absolute valued pathway enrichment analysis yields pathways with genes that might be changed in both directions (both over-expressed and under-expressed) because it estimates enrichment in the differentially expressed tail of the signature (irrespective of sign). Such absolute valued pathway enrichment analysis provided NESs for each of 833 pathways, as described above. An “absolute valued” pathway enrichment analysis was performed using the differential methylation signature of treatment response in the similar manner.

The next step was to then integrate NESs from signed and absolute valued pathway enrichment analysis such that, for each pathway, a final integrative NES was defined as an NES with the most significant p-value between the signed and absolute valued pathway analysis (negative NES values for absolute valued analysis were not considered because they reflect enrichment in the non-changed part of the signature). The advantage of such an integration is two-fold: it captures (1) pathways with genes that are strictly over-expressed or under-expressed in each pathway and (2) pathways with genes that are significantly changed in both directions (i.e., pathways that include genes that are significantly over-expressed and genes that are significantly under-expressed). Thus, the integration increases the probability of identifying functionally relevant molecular determinants. Such an integration of signed and absolute valued NESs provides a composite expression pathway signature and a composite methylation pathway signature.

Genomic and epigenomic pathway integration: To identify pathways that are significantly affected on both genomic and epigenomic levels, GSEA was employed to compare composite expression pathway signatures and composite methylation pathway signatures to identify pathways that are significantly affected on both genomic and epigenomic levels (pathways that belong to the leading edge of the GSEA analysis). To ensure identification of pathways that are (i) over-expressed and under-methylated, (ii) under-expressed and over-methylated, and (iii) differentially expressed and differentially methylated, each pathway signature was ranked based on the absolute values of their NESs and used for a subsequent GSEA comparative analysis.

For this pathway-based GSEA, a composite expression pathway signature was used as a reference signature, and top pathways from the composite methylation pathway signature were used as a query pathway set. To accurately define a query pathway set that ensures the most significant enrichment between pathway signatures, the threshold for the query pathway set was varied between 0.001 and 0.05 (width of each step=0.005), and the strength of enrichment between the two signatures was estimated at each threshold. For each threshold, GSEA was run 100 times, and the average NES for the enrichment was reported. The threshold with the highest average NES then reflects the optimal threshold that corresponds to the most significant enrichment between the composite expression pathway signature and the composite methylation pathway signature and was used for subsequent analysis. GSEA analyses between the composite expression pathway signature and the composite methylation pathway signature at the optimal threshold identified a set of 28 pathways of treatment response, which were significantly altered on both genomic and epigenomic levels.

One of the limitations of the pathways from the C2 collection is that they often represent a parent-child relationship, where a parent pathway (such as a cell cycle) would encompass all genes in child pathways (such as cell cycle phase). Such overlap produces data redundancy and can result in model overfitting as the “same” pathways are fit in the model repeatedly. To overcome this limitation and to eliminate pathways with heavy overlaps, a Fisher Exact Test (Fisher R A, Journal of the Royal Statistical Society. 1922; 85(1):87-94) (fisher.test function in R) was performed, and leading edge genes for each pair of pathways from the analysis were compared (for all 28 pathways, which resulted in [28 choose 2=378] comparisons). From each group of parent-children pathways that shared a large number of overlapping genes, one representative pathway was selected with the most significant NES, which defined a final set of seven (7) maximally non-overlapping non-redundant pathways used for subsequent analysis.

Evaluating expression and methylation data in the integrative analysis: To examine if both data types (mRNA expression and DNA methylation) from the 7 candidate pathways have the equivalent ability to predict a therapeutic response, the performance of the 7 pathways was compared utilizing only their (i) activity levels based on expression and (ii) activity levels based on methylation, separately. To compare pathway performances based on each data type, both expression and methylation data matrices (z-scored on genes) were scaled in the TCGA-LUAD cohort, which defined single-sample differential expression and single-sample differential methylation signatures, respectively. Each sample was then used for signed and absolute valued pathway enrichment analysis (separately for expression and for methylation, as above), in which each single-sample signature was used as a reference, and genes from each of 7 candidate pathways were used as a query set, thus, producing a pathway activity signature for each patient. These single-sample expression and methylation pathway signatures were then used to evaluate the predictive ability of 7 pathways (for expression and methylation, separately) using logistic regression modeling (Walker et al., Biometrika. 1967; 54(1/2):167-79) followed by Receiver Operating Characteristic (ROC) analysis (Metz C E,—Seminars in nuclear medicine. 1978; 8(4):283-98). Here, the area under ROC (AUROC) reflected how well each data type separates poor-response and favorable-response patients in the TCGA-LUAD patient cohort (the AUROC value of 0.5 indicates a random predictor, and 1 indicates a perfect predictor). The logistic regression analysis was done using glm (Chambers et al., Statistical Models in S1990; Heidelberg: Physica-Verlag HD) function and ROC analysis was done using pROC (Robin X et al., BMC bioinformatics. 2011; 12(1):77) and ggplot2 (Wickham, J Stat Softw. 2010; 35(1):65-88) package in R.

Validation and robustness in independent clinical cohorts: To evaluate clinical significance of the 7 candidate molecular pathways, their ability to predict patients at risk of chemoresistance was examined in an independent clinical cohort from the Tang et al. (Tang et al., Clin. Can. Res. 2013; 19(6):1577-86) dataset, and survival status was used during the clinical study (1996 to 2007) as a clinical endpoint (time to event or follow-up was estimated between the start of carboplatin-paclitaxel treatment and death or follow-up, respectively; maximum time to event/follow-up is 2,567 days).

First, activity levels of the 7 candidate pathways in the Tang et al. cohort were estimated on a single-sample level, as above. The activity levels (NESs) of the 7 candidate pathways were then subjected to t-Distributed Stochastic Neighbor Embedding (t-SNE) clustering (Maaten Lvd et al., Journal of machine learning research. 2008; 9(November):2579-605) (implemented through Rtsne (Maaten L V D., J Mach Learn Res. 2014; 15(1):3221-45) package in R), a non-linear dimensionality reduction technique which chooses two similarity measures between pairs of points of (i) high dimensional input space and (ii) low-dimensional embedding space. First, it constructs a probability distribution over the pairs of high dimensional space (7-dimension in this case) in such a way that similar points are exhibited by nearby instances, while dissimilar points are exhibited by distant instances. Second, it constructs a similar probability distribution over the points in low-dimensional embedding space and tries to minimize the Kullback-Leibler divergence (KL divergence) (Kullback et al., Ann Math Statist. 1951; 22(1):79-86) between the high dimensional data and low dimensional anticipated data at each point. Therefore, patients with similar pathway activity levels will be anticipated as nearby instances, while patients with dissimilar pathway activity levels will be anticipated as dissimilar instances. The advantage of t-SNE lies in its ability to reduce dimensions from seven (maximum possible in the analysis) to two and effectively identify groups of patients that share similar pathway activity levels. This analysis stratified patients into two groups: a group with overall increased composite pathways' activities and a group with overall decreased composite pathways' activities. Next, whether or not these patient groups significantly differ in their response to carboplatin-paclitaxel treatment was examined using a Kaplan-Meier survival analysis (Kaplan et al., Journal of the American Statistical Association. 1958; 53(282):457-81) and Cox proportional hazards model (Cox D R., Journal of the Royal Statistical Society Series B (Methodological). 1972; 34(2):187-220) via survival (Therneau T., A package for survival analysis in S. R package version 2.38. Retrieved from CRAN R-project org, 2015), ggplot2 (Wickham, J Stat Softw. 2010; 35(1):65-88), and survminer (Kassambara et al., survminer: drawing survival curves using ‘ggplot2’. R package version 0.2. 4. 2016) R packages.

In order to evaluate whether a random set of pathways can perform as well as the identified 7 pathways, the predictive ability of the 7 candidate pathways was compared with the predictive ability of 7 pathways selected at random. For this analysis, a random model was constructed, in which 7 pathways were selected at random, and their activity levels were utilized to stratify patients based on their treatment response with a subsequent evaluation using a Kaplan-Meier survival analysis. Random selection was performed 10,000 times, and the empirical p-value was estimated as the number of times a Kaplan-Meier log-rank p-value for 7 candidate molecular pathways outperformed the results at random. Also employed was a second random model, in which the effect of selecting random patient groups was evaluated.

Finally, to estimate the accuracy with which the systems and methods disclosed herein can predict a treatment response for a new incoming patient, this process was simulated using leave-one-out cross-validation (LOOCV) (Stone M., Journal of the royal statistical society Series B (Methodological). 1974:111-47). In LOOCV, one patient is “removed”, and the model is trained on the rest of the patients. The patient that was removed is considered a new incoming patient, subjected to predictive analysis, and assigned a risk of developing resistance. This process was repeated for all patients. The predictive model for LOOCV was implemented using generalized linear modeling (such as multivariable logistic regression) through the glm (Chambers et al., Statistical Models in S1990; Heidelberg: Physica-Verlag HD) function and ggplot2 (Wickham,-J Stat Softw. 2010; 35(1):65-88) package in R.

Comparison to other methods, common covariates, and signatures of aggressiveness: To assess exemplary advantages of the systems and methods disclosed herein, (i) its predictive performance was compared to other commonly utilized approaches, including linear regression modeling, support vector machine, and random forest; and (ii) whether or not the method can be affected by commonly used covariates or known signatures of lung cancer aggressiveness was evaluated.

First, to demonstrate exemplary advantages of the systems and methods disclosed herein over other commonly utilized approaches, performance of these systems and methods was compared with (i) Panja et al. (Panja et al., EBioMedicine. 2018), Epigenomic and Genomic mechanisms of treatment Resistance (Epi2GenR), which uses linear regression to integrate DNA methylation and mRNA expression data; (ii) Zhong et al. (Zhong et al., Scientific reports. 2018; 8(1):12675), which is based on a support vector machine (SVM) algorithm that uses patient mRNA expression profiles; and (iii) Yu et al. (Yu et al., Scientific reports. 2017; 7:43294), Personalized REgimen Selection (PRES) method, which is based on a random forest machine learning approach that uses patient mRNA expression profiles. The selection and cross-validation techniques were followed as suggested in each of the above publications to carefully compare their performance to the systems and methods disclosed herein. Epi2GenR utilized the same signature as utilized in these Examples 1 and 2. To apply SVM and PRES correctly, the validation set was split into 70:30 proportion subsets, in which 70% of the validation set was used for model training, and 30% was used for model validation. The predictive ability of the identified candidates from each method was evaluated using ROC, Kaplan-Meier survival, and hazard ratio analyses through the survival (Therneau T., A package for survival analysis in S. R package version 2.38. Retrieved from CRAN R-project.org, 2015), survcomp (Schroder M S, et al., Bioinformatics 2011; 27(22):3206-8), and survminer (Kassambara et al., survminer: drawing survival curves using ‘ggplot2’. R package version 0.2. 4. 2016) packages in R.

Second, whether any of the commonly used covariates (such as age, gender, and tumor stage at diagnosis) and known signatures of lung cancer aggressiveness (such as from Larsen et al. (Larsen et al., Clin. Can. Res. 2007; 13(10):2946-54), Beer et al. (Beer et al., Nature medicine. 2002; 8(8):816-24), and Tang et al. (Tang et al., Clin. Can. Res. 2013; 19(6):1577-86) described above) can predict a therapeutic response or can significantly affect the predictive ability of the identified 7 candidate pathways was evaluated. For this analysis, the multivariable Cox proportional hazards model (Cox D R., Journal of the Royal Statistical Society Series B (Methodological). 1972; 34(2):187-220) (using coxph function) and stratified Kaplan-Meier survival analysis (Kaplan et al., Journal of the American Statistical Association. 1958; 53(282):457-81) were used through the survival (Therneau T., A package for survival analysis in S. R package version 2.38. Retrieved from CRAN R-project.org, 2015), and survminer (Kassambara et al., survminer: drawing survival curves using ‘ggplot2’. R package version 0.2. 4. 2016) packages in R.

Model generalizability: To test the generalizability of the model, the systems and methods disclosed herein were applied to additional chemotherapy combinations (such as cisplatin-vinorelbine and oxaliplatin-fluorouracil) and additional cancer types (such as lung squamous cell carcinoma and colorectal adenocarcinoma). The investigations included the response to (i) cisplatin (platinum-based alkylating chemotherapy) and vinorelbine (non-platinum based plant alkaloid chemotherapy) response in lung adenocarcinoma (LUAD); (ii) cisplatin-vinorelbine response in lung squamous cell carcinoma (LUSC); and (iii) oxaliplatin (platinum-based alkylating chemotherapy), fluorouracil (antimetabolite chemotherapy), and folinic acid (chemotherapy protective drug often given with fluorouracil to improves the binding; also known as leucovorin) (FOLFOX) response in colorectal adenocarcinoma (COAD).

For signature development, primary tumor samples from TCGA-LUAD/TCGA-LUSC/TCGA-COAD (n=8) were used for patients without neo-adjuvant treatment (no pre-treatment), who received adjuvant chemotherapies of interest and were further monitored for new tumor events (as defined above). As in the TCGA cohorts above, mRNA expression (RNA seq) was profiled using an Illumina HiSeq 2000, and DNA methylation was profiled using an Illumina Infinium Human Methylation (HM450) array.

For clinical validation of the cisplatin-vinorelbine combination response in LUAD, the Zhu et al. patient cohort (Zhu et al., J. Clin. Oncol. 010; 28(29):4417-24) (GSE14814) was used, which included LUAD tumors obtained at surgery (n=39), treated with adjuvant cisplatin-vinorelbine chemotherapy, and profiled on Affymetrix Human Genome U133A platform. In this cohort, lung cancer-related death was used as a clinical endpoint, and time to event was calculated between the start of cisplatin-vinorelbine treatment and lung-cancer related death (for patients with this event) or to follow-up (for censored patients) with the maximum time to event/follow-up at 3,390 days.

For clinical validation of the cisplatin-vinorelbine combination response in lung squamous cell carcinoma (LUSC), a different subset of patients from the Zhu et al. patient cohort (Zhu et al., J. Clin. Oncol. 2010; 28(29):4417-24) (GSE14814) was used, which included patients with LUSC, whose tumors were obtained at surgery (n=26) and who were treated with adjuvant cisplatin-vinorelbine chemotherapy and profiled on Affymetrix Human Genome U133A platform. In this cohort, lung cancer-related death was used as a clinical endpoint, and the time to event was calculated between the start of cisplatin-vinorelbine treatment and lung-cancer related death (for patients with this event) or to follow-up (for censored patients) with the maximum time to event/follow-up at 3,318 days.

Finally, for validation of the FOLFOX combination in colorectal adenocarcinoma (COAD), the Marisa et al. patient cohort (Marisa et al., PLoS medicine. 2013; 10(5):e1001453) (GSE39582) was used, which includes COAD tumors obtained at surgery (n=23), treated with adjuvant FOLFOX chemotherapies, and profiled on Affymetrix Human Genome U133 Plus 2.0 Array. In this cohort, relapse-free survival (where relapse was defined as locoregional or distant recurrence) was used as a clinical endpoint, and time to event was calculated between the start of FOLFOX treatment to relapse (for patients with this event) or to follow-up (for censored patients), with the maximum time to event/follow-up at 2,790 days. The clinical characteristics of all subjects are summarized in Table 5.

### Example 2—Results

Systems and methods for genome-wide computation were developed that can integrate mRNA expression and DNA methylation patient profiles to identify pathways altered at both genomic and epigenomic levels (as demonstrated in FIG. 1) that differentiate poor and favorable responses to chemotherapy regimens. Here, steps included in the integrative systems and methods (also shown in Tables 2-5) are provided. Step 1: two groups of patients are identified, which are used to define a “chemotherapy response signature”: (i) patients that failed a specific chemotherapy regimen (such as patients that developed metastasis within 1 year after therapy administration) and (ii) patients with a favorable chemotherapy response (such as patients that remained disease-free for more than 2 years after chemotherapy administration). Step 2: genomic (mRNA expression) and epigenomic (DNA methylation) profiles are compared between the two groups of patients, which define differential (i) genomic signature and (ii) epigenomic signature of chemoresponse. Step 3: such signatures are individually subjected to signed and absolute valued pathway enrichment analysis, which yields molecular pathways enriched in the genomic signature (composite pathways with genes that are differentially expressed) and pathways enriched in the epigenomic signature (composite pathways with genes are differentially methylated). Step 4: The composite genomic and epigenomic pathway signatures are then integrated to determine a set of pathways that control both genomic and epigenomic programs that are disrupted in resistance. Step 5: candidate pathways are subjected to validation studies, in which they are evaluated for their ability to predict therapeutic response in independent patient cohorts through a multivariable survival analysis. Step 6: finally, the identified pathways are used to assign individual risk of resistance for new incoming patients.

Defining epigenomic signatures of chemotherapy response: The systems and methods were applied to evaluate the response to standard-of-care chemotherapy combination carboplatin and paclitaxel (carboplatin-paclitaxel) in LUAD patients. For this analysis, clinical and molecular profiles of patients with LUAD in the TCGA clinical cohort were analyzed (Cancer Genome Atlas Research Network, Nature. 2014; 511(7511):543-50). To study primary resistance to this chemo combination, patients were selected that did not receive neoadjuvant therapy, were treated with adjuvant carboplatin-paclitaxel chemo regimen, and were further monitored for disease progression (n=14) (Table 2). Each patient that received carboplatin-paclitaxel was evaluated for his/her time to tumor relapse, which was defined as the time between the start of carboplatin-paclitaxel administration and a new tumor event (defined as tumor reappearance or local or distant metastases). To accurately determine a signal that differentiates poor from favorable treatment responses, responder and non-responder analyses were used (such as in Panja et al., EBioMedicine. 2018; 31:110-121), and the tails of the therapeutic response distributions were compared to capture the most prominent molecular signal that differentiates these treatment response groups. To ensure that the comparison groups were balanced with respect to initial age, gender, tumor aggressiveness, smoking status, etc., stratified sub-sampling was performed (which identifies patient groups with similar distributions for these variables), and patients that experienced relapse within 1 year of carboplatin-paclitaxel start (poor response, n=4) as well as patients that did not experience any events for more than 2 years (favorable response, n=4) were identified (Table 2).

To uncover a complex interplay between genomic and epigenomic mechanisms implicated in response to chemotherapy, poor response and favorable response groups were compared based on mRNA expression and DNA methylation profiles using two-sample two-tailed Welch t-test (Welch, Biometrika. 1947; 34(1-2):28-35) (see Example 1), which yielded a carboplatin-paclitaxel response differential gene expression signature and carboplatin-paclitaxel response differential methylation signature. Top differentially expressed genes in the carboplatin-paclitaxel response differential gene expression signature included WWC3, which is a therapeutic target in lung cancer (Han et al., OncoTargets and therapy. 2018; 11:2581-91); CDR1, which is a biomarker in prostate cancer (Salemi et al., The International journal of biological markers. 2014; 29(3):e288-90); FCGBP, which is a potential therapeutic target in metastatic colorectal cancer (Qi et al., Oncology Letters. 2016; 11(1):568-74); and DPYSL2, PTK2 (Bhattacharjee et al., Proceedings of the National Academy of Sciences of the United States of America. 2001; 98(24):13790-5), and DUSP6 (Chen et al., Journal of the National Cancer Institute. 2011; 103(24):1859-70), which are prognostic markers of lung cancer. Genes that harbored top differentially methylated sites in the carboplatin-paclitaxel response differential methylation signature included hypermethylated LAMB3, which is a biomarker of lung cancer (Belinsky S A., Nature reviews Cancer. 2004; 4(9):707-17); CD63, which is a predictive biomarker of LUAD (Kwon et al., Lung cancer. 2007; 57(1):46-53); HES4, which is a prognostic biomarker of osteosarcoma (McManus et al., Pediatric blood & cancer. 2017; 64(5); DAXX, which is a therapeutic target in metastatic lung cancer (Lin et al., Nature Communications. 2016; 7:13867); TSPO, which is a molecular target for tumor imaging and chemotherapy (Austin et al., The international journal of biochemistry & cell biology. 2013; 45(7):1212-6); REG1A, H2AFZ (Beer et al., Nature medicine. 2002; 8(8):816-24), POLG2 (Larsen et al., Clin. Can. Res. 2007; 13(10):2946-54), TOM1L1 (Bhattacharjee et al., Proceedings of the National Academy of Sciences of the United States of America. 2001; 98(24):13790-5) and MB (Zhu et al., J. Clin. Oncol. 2010; 28(29):4417-24), which are known prognostic markers of lung cancer.

Integrative analysis identified epigenomic pathways implicated in resistance: To understand molecular mechanisms that govern chemoresponse, molecular pathways that control genomic and epigenomic signatures of carboplatin-paclitaxel resistance were identified. For this analysis, the carboplatin-paclitaxel response differential expression signature and carboplatin-paclitaxel response differential methylation signature were subjected to a pathway enrichment analysis using the C2 pathway database (which includes the REACTOME (Fabregat et al., Nucleic Acids Res. 2016; 44(D1):D481-7), KEGG (Ogata et al., Nucleic Acids Res. 1999; 27(1):29-34), and BIOCARTA (Nishimura D., Biotech Software & Internet Report: The Computer Software Journal for Scient. 2001; 2(3):117-20) pathways). Pathway enrichment was performed using Gene Set Enrichment Analysis (GSEA) (Subramanian et al., PNAS. 2005(102):15545-50), in which each pathway is assigned a score (i.e., Normalized Enrichment Score, NES) that reflects the level of enrichment in the signature of resistance, also referred to as pathway activity, for the pathway. A list of 833 pathways ranked by their enrichment (NESs) in the carboplatin-paclitaxel response differential expression signature was used to determine the carboplatin-paclitaxel response differential expression pathway signature, and a list of 833 pathways ranked by their enrichment (NESs) for the carboplatin-paclitaxel response methylation signature were used to determine the carboplatin-paclitaxel response differential methylation pathway signature (see Methods). To account for the pathways that have majority of their genes affected in the same direction (such as over-expressed or under-expressed) and pathways that have some genes affected in one direction (such as over-expressed) and some in an opposite direction (such as under-expressed), both signed and absolute valued pathway enrichment analyses were performed with subsequent integration (see Example 1), which were used to determine the carboplatin-paclitaxel response composite expression pathway signature and carboplatin-paclitaxel response composite methylation pathway signature.

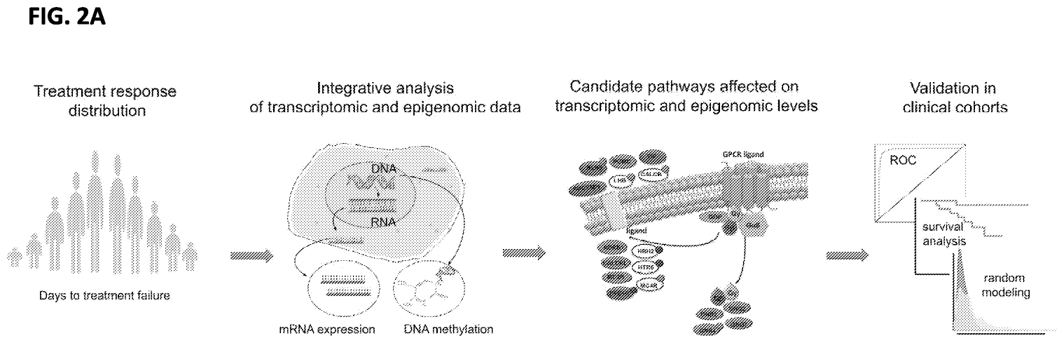

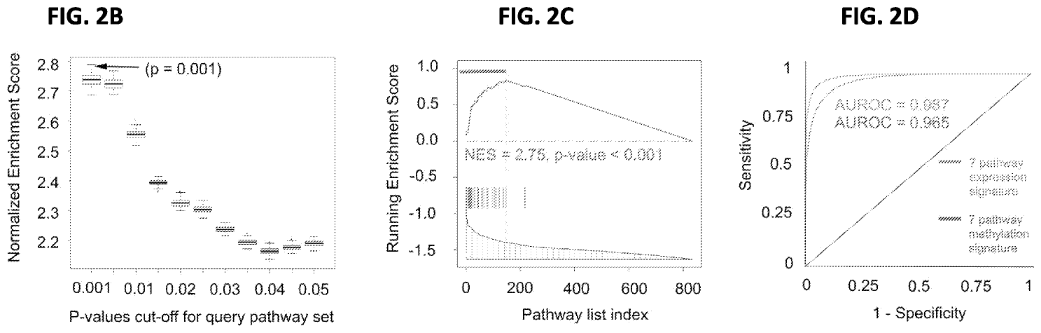

Further, to determine interplay between complex mechanisms implicated in chemoresistance, molecular pathways were identified that are affected at both genomic (such as mRNA expression) and epigenomic (such as DNA methylation) levels and that capture pathways with genes affected (i) only at the genomic level, (ii) only at the epigenomic level, (iii) or at both levels (as in FIG. 1). To achieve this goal (FIG. 2A), the carboplatin-paclitaxel response composite expression pathway signature and carboplatin-paclitaxel response composite methylation pathway signature were compared using GSEA, where the carboplatin-paclitaxel response composite expression pathway signature was used as a reference and the carboplatin-paclitaxel response composite methylation pathway signature was used as a query pathways set (the threshold for the query pathway was p-value<0.001 as shown in FIG. 2B, Example 1), which were used to identify 7 molecular pathways with significant alterations at both genomic and epigenomic levels (NES=2.75, p-value<0.001) (FIG. 2C, Example 1). The pathways include (i) chemokine receptors bind chemokines (Fabregat et al., Nucleic Acids Res. 2016; 44(D1):D481-7), (ii) mRNA splicing (Fabregat et al., Nucleic Acids Res. 2016; 44(D1):D481-7), (iii) G alpha signaling events (Fabregat et al., Nucleic Acids Res. 2016; 44(D1):D481-7), (iv) intestinal immune network for IgA production (Ogata et al., Nucleic Acids Res. 1999; 27(1):29-34), (v) metabolism of proteins (Fabregat et al., Nucleic Acids Res. 2016; 44(D1):D481-7), (vi) RNA degradation (Ogata et al., Nucleic Acids Res. 1999; 27(1):29-34), and (vii) cell cycle mitotic (Fabregat et al., Nucleic Acids Res. 2016; 44(D1):D481-7).

To investigate whether mRNA expression or DNA methylation carries a more significant weight in the predictive ability of the 7 candidate pathways, a ROC analysis was performed based on pathway activities in each patient sample defined on either (i) expression levels or (ii) methylation levels of the pathway genes (Example 1). The analysis demonstrated that both expression levels (AUROC=0.987) and methylation levels (AUROC=0.965) of 7 candidate pathways are highly predictive of poor response vs favorable response separation (FIG. 2D), indicating that they both can be used to identify patients at risk of developing chemoresistance.

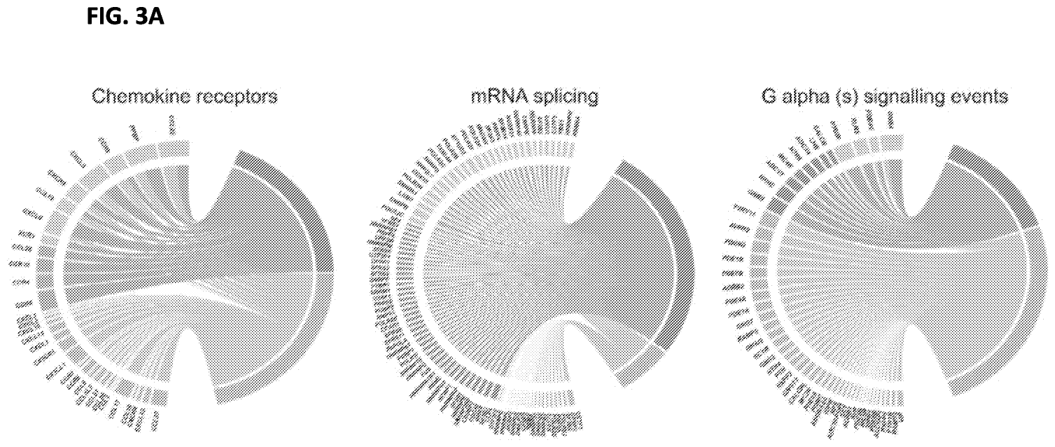

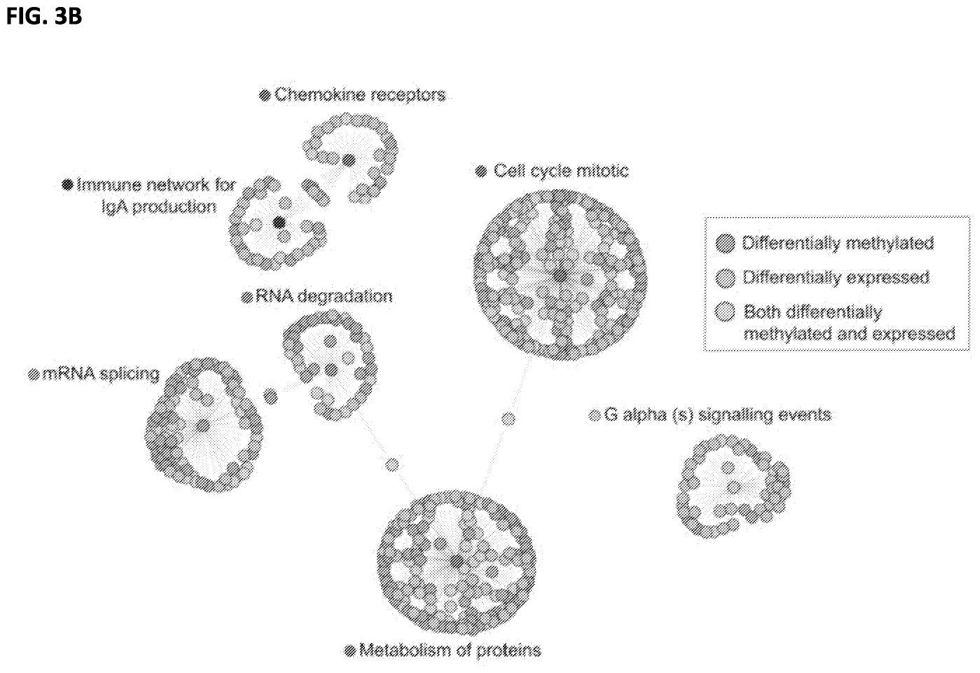

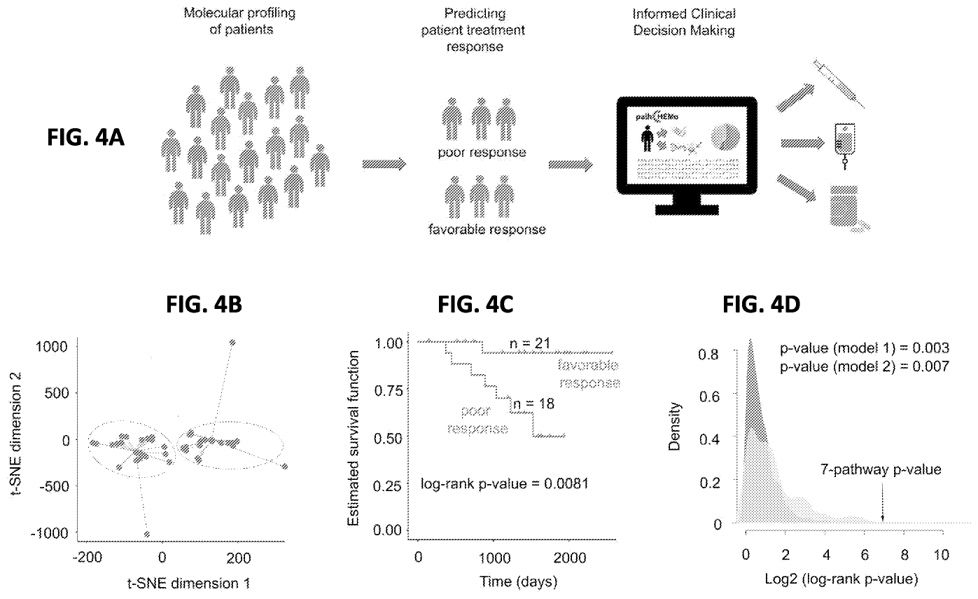

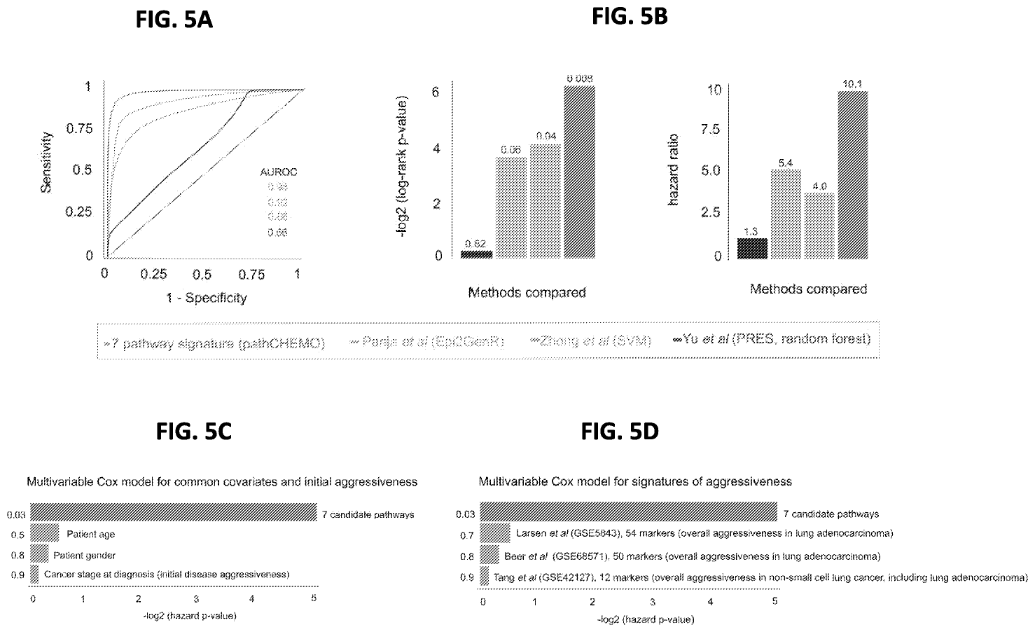

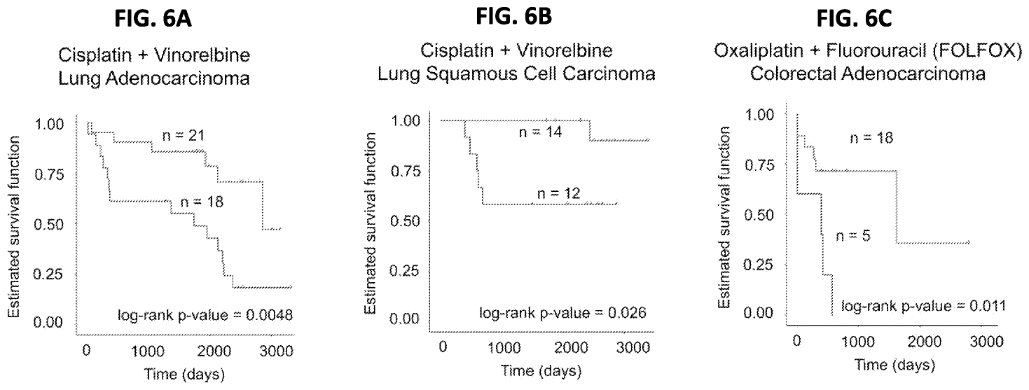

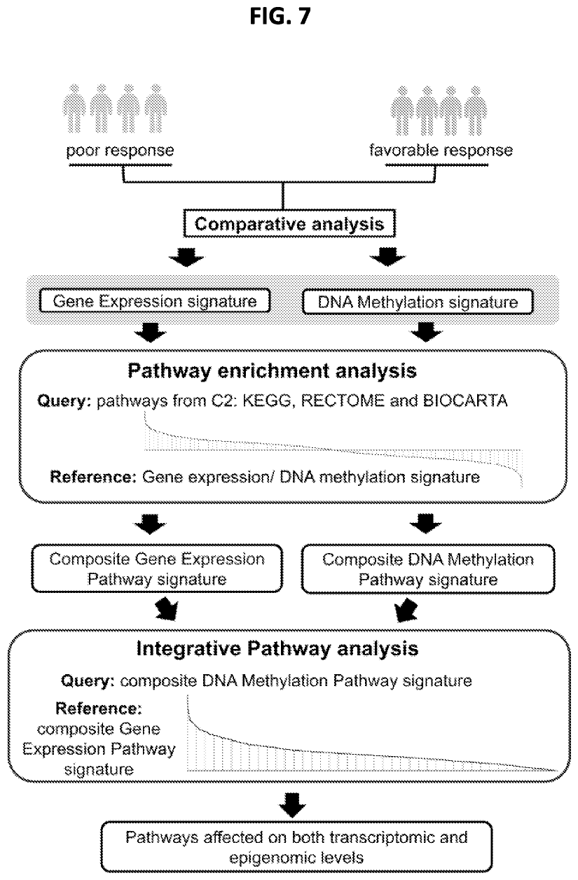

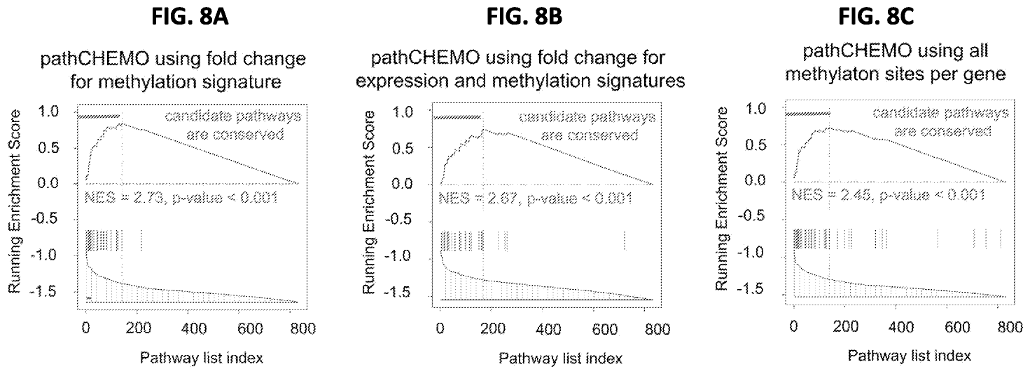

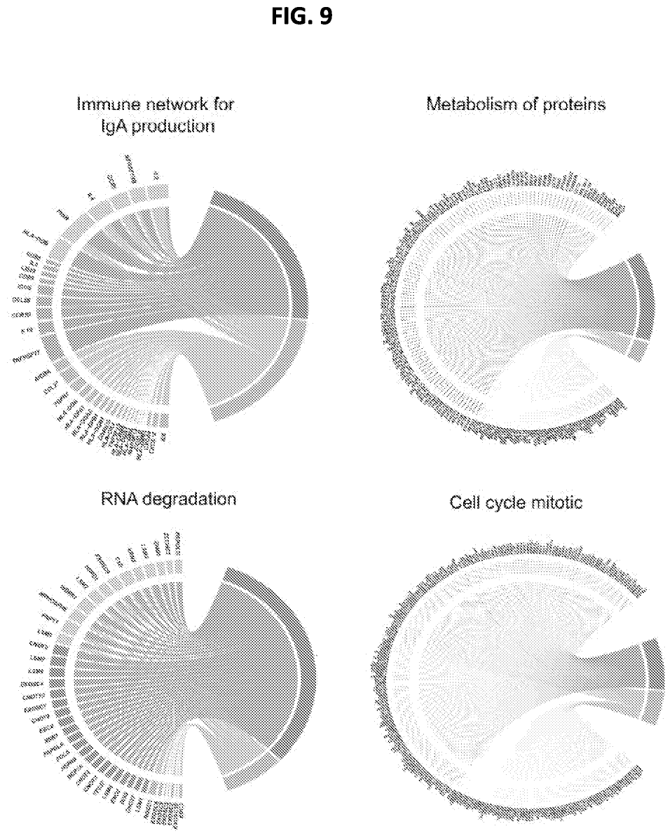

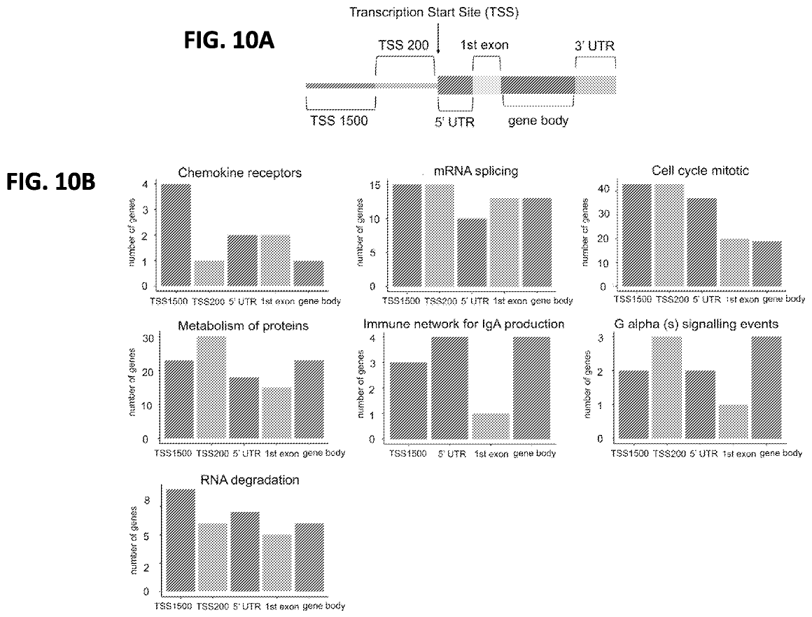

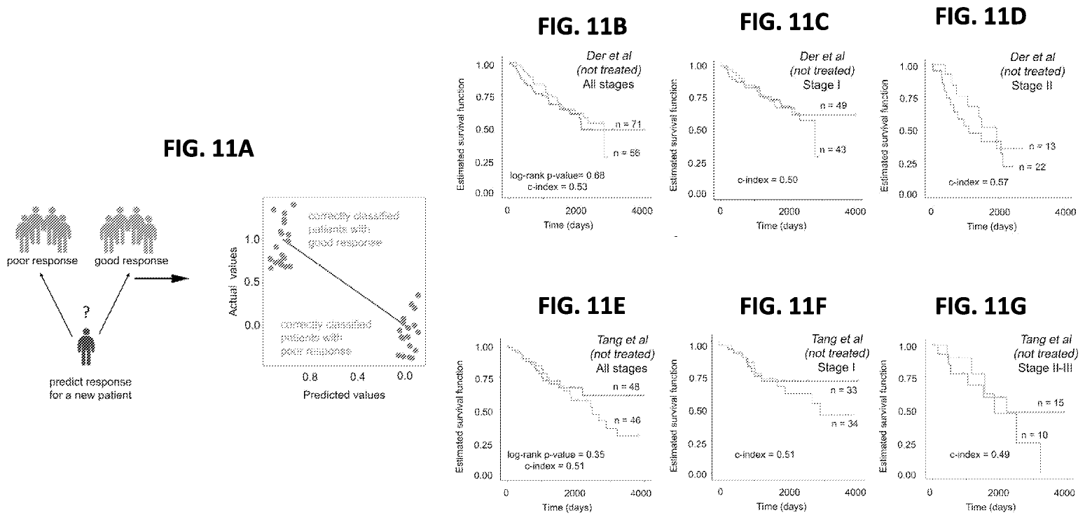

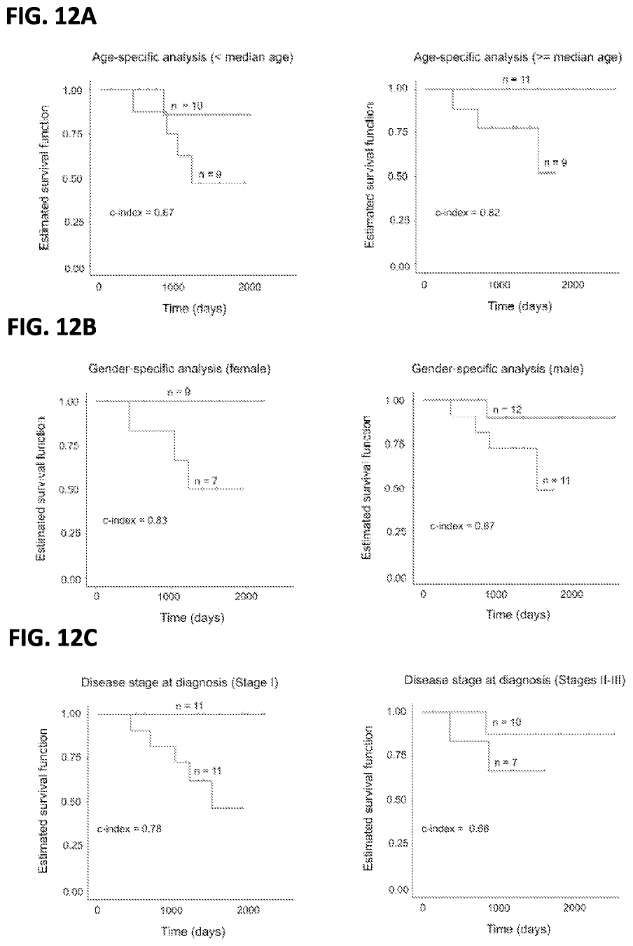

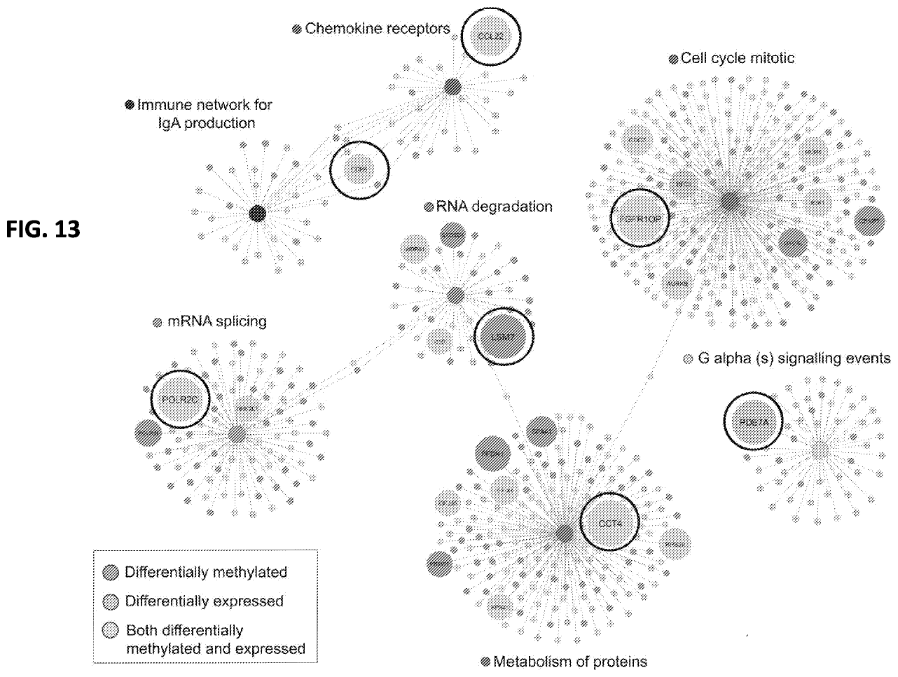

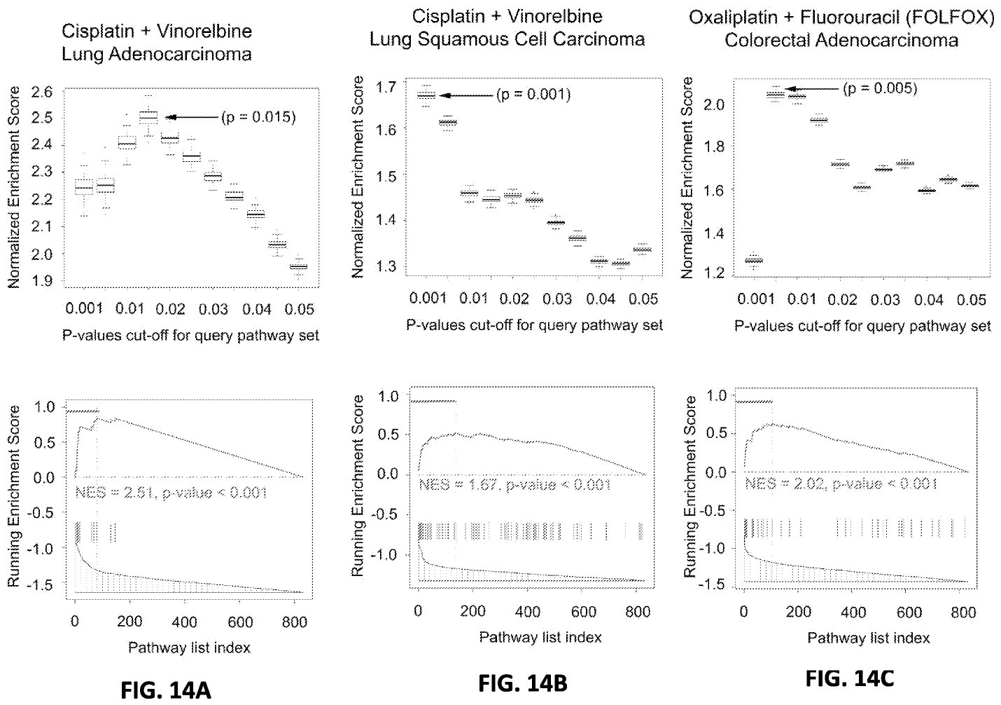

Further evaluated was a topological structure of genomic and epigenomic alterations within each identified pathway. First, the extent to which genes from each pathway were affected on genomic or on epigenomic levels was evaluated (FIG. 3A, FIGS. 8A-8C), and 7 pathways exercised different patterns of genomic and epigenomic alterations. For example, majority of genes from the G alpha signaling events pathway were altered at the mRNA level (FIG. 3A, nodes in pink), while genes from the mRNA splicing pathway were heavily altered at the DNA methylation level (FIG. 3A, nodes in grey) and at both mRNA expression and DNA methylation levels (FIG. 3A, nodes in yellow). Second, connectivity was examined within and between the pathway genes, in which an edge within the pathway corresponds to the pathway membership and a connecting edge between pathways shows shared genes and demonstrates that the candidate pathways share little overlap (FIG. 3B). Finally, differentially methylated sites harbored in genes from the 7 pathways were examined and their regions/locations on the genome were evaluated (FIG. 10A), in which regions were defined as TSS200 (200 base pairs upstream of transcription start site, TSS), TSS1500 (1500 base pairs upstream of TSS200), 5′UTR, 1st exon, gene body, and 3′UTR. In fact, the majority of pathways have methylated sites overrepresented in TSS200+TSS1500 regions, indicating a possible interaction with the transcription machinery binding at the promoter/enhancer regions (Zhang et al., Nucleic Acids Res. 1986; 14(21):8387-97). An exception was the Immune network for IgA production pathway, in which sites were heavily enriched in the gene body, indicating their potential interaction with alternative splicing machinery (Laurent et al., Genome research. 2010; 20(3):320-31) (FIG. 10B).

Validation in independent patient cohorts: The next step was to evaluate if the candidate molecular pathways can stratify patients based on the risk of failing chemotherapy in an independent, non-overlapping patient cohort (FIG. 4A). For this analysis, the Tang et al. cohort (Tang et al., Clin. Can. Res. 2013; 19(6):1577-86) (Table 2) from the University of Texas MD Anderson Cancer Center was considered, which contains LUAD tumor samples obtained at surgery (n=39) collected between 1996 to 2007, followed by treatment with carboplatin and a taxane (e.g., paclitaxel), and monitored for further disease progression for 11 years. In this cohort, survival status during the clinical study (1996 to 2007) was used as a clinical endpoint, and the time to this event was calculated between the start of carboplatin-paclitaxel treatment to death (for patients with this event) or to follow-up (for censored patients). Similar to the analysis above, activity levels of 7 candidate pathways in each patient sample were evaluated (such as through a single-sample pathway analysis, Example 1), and t-SNE clustering was employed, which stratified patients into two groups based on pathway activity levels (FIG. 4B): one group with increased composite pathways' activities (orange) and one group with decreased composite pathways' activities (green). These patient groups were then subjected to a Kaplan-Meier survival analysis (Kaplan et al., Journal of the American Statistical Association. 1958; 53(282):457-81) and a Cox proportional hazards model (Cox D R., Journal of the Royal Statistical Society Series B (Methodological). 1972; 34(2):187-220) (FIG. 4C), which demonstrated a significant difference in the groups' responses to carboplatin-paclitaxel (log-rank p-value=0.0081, hazard ratio=10) (Example 1).

To evaluate the non-randomness of this result, the predictive ability of the 7 candidate pathways was compared with the predictive ability of 7 pathways selected at random (Example 1), which demonstrated that the candidate 7 pathways predict the carboplatin-paclitaxel response non-randomly compared with 10,000 randomly selected pathways (FIG. 4D, random model 1: p-value=0.003). This analysis paralleled and evaluation of whether patient groups stratified by the model showed a significantly different treatment response compared with patient groups chosen at random, which were shown to be non-random (FIG. 4D, random model 2: p-value=0.007).

Further, a situation was simulated in which a new incoming patient is diagnosed with LUAD and needs to be assigned risk of developing resistance to carboplatin-paclitaxel utilizing leave-one-out cross-validation (LOOCV) (Stone M., Journal of the royal statistical society Series B (Methodological). 1974:111-47) in the Tang et al. validation cohort. (Tang et al., Clin. Can. Res. 2013; 19(6):1577-86). In LOOCV, one patient is “removed”, and the model is trained on the rest of the patients. The patient that was removed is subjected to predictive analysis and is assigned a risk of developing resistance (simulating a scenario of a new incoming patient). This process was repeated for all patients (Example 1). The LOOCV analysis demonstrated that the systems and methods disclosed herein exhibit high accuracy at predicting poor and favorable carboplatin-paclitaxel responses for new incoming patients (FIG. 11A).

Finally, to show that the candidate pathways distinguish carboplatin-paclitaxel response and not disease aggressiveness, whether the pathways can also separate patients based on their lung cancer aggressiveness was examined. For this analysis, the predictive ability of the candidate pathways was examined for the LUAD patient cohorts that did not receive treatment after surgery (these cohorts were considered negative controls). The datasets (FIG. 7) included (i) Der et al. (Der et al., J. Thor. Oncol. 2014; 9(1):59-64) LUAD tumor samples (n=127) collected through surgery between 1996 to 2005 at Princess Margaret Cancer Centre and (ii) the Tang et al. (Tang et al., Clin. Can. Res. 2013; 19(6):1577-86) provisional cohort, which includes LUAD tumor samples (n=94) collected through surgery between 1996 to 2007 at The University of Texas MD Anderson Cancer Center. These negative control patient cohorts did not receive subsequent treatment but were monitored for disease progression (for Der et al., lung cancer-related death was used as a clinical endpoint, and, for Tang et al., survival status during clinical study (1996 to 2007) was used as a clinical endpoint). A Kaplan-Meier survival analysis on these datasets demonstrated that the candidate 7 pathways did not separate patients based on the disease progression in both unstratified and stratified (based on tumor stages) analyses, (i) Der et al. (FIGS. 11B-11D, log-rank p-value=0.68) and (ii) Tang et al. (FIGS. 11E-11G, log-rank p-value=0.35); thus, the 7 candidate pathways are specific for a carboplatin-paclitaxel response.

Comparison to other methods, signatures of aggressiveness, and common covariates: To assess the advantages of the systems and methods herein, (i) the predictive performance was compared with other commonly utilized approaches, including methods based on linear regression modeling, support vector machine (SVM), and random forest; and (ii) whether the systems and methods disclosed herein can be affected by commonly utilized covariates or known signatures of lung cancer aggressiveness was examined.

First, to measure the advantage of the systems and methods disclosed herein over other commonly utilized methods, the predictive performance of the systems and methods disclosed herein was compared (Example 1) with (i) Panja et al. (Panja et al., EBioMedicine. 2018; 31:110-121), Epi2GenR based on linear regression integration between DNA methylation and mRNA expression patient profiles, which identified 35 site-gene pairs as candidate markers of carboplatin-paclitaxel response; (ii) Zhong et al. (Zhong et al., Scientific reports. 2018; 8(1):12675), based on a support vector machine (SVM) analysis, which identified 104 candidate genes; and (iii) Yu et al. (Yu et al., Scientific reports. 2017; 7:43294), PRES, based on a random forest algorithm, which identified 3 candidates for the carboplatin-paclitaxel response. The abilities of the identified candidates from each method to separate patients with poor and favorable carboplatin-paclitaxel responses were compared using the Tang et al. dataset and an ROC analysis, which demonstrated the advantage of pathCHEMO over other commonly utilized methods (FIG. 5A, AUROCpathCHEMO=0.98, AUROCEpi2GenR=0.92, AUROCSVM=0.86, AUROCPRES=0.66). Furthermore, the ability of these methods to predict responses to carboplatin-paclitaxel was compared using the Tang et al. validation set, as above, and a Kaplan-Meier survival analysis (FIG. 5B (left), log-rank p-valuepathCHEMO=0.008, log-rank p-valueEpi2GenR=0.04, log-rank p-valueSVM=0.06, log-rank p-valuePRES=0.82) as well as a Cox proportional hazards model (FIG. 5B (right), hazard ratiopathCHEMO=10.1, hazard ratioEpi2GenR=4.0, hazard ratioSVM=5.4, hazard ratioPRES=1.3), which confirmed that, for the Tang et al. validation set, pathCHEMO outperformed other commonly used methods in the ability to predict a therapeutic response.

Second, to ensure that the model is not affected by commonly utilized covariates (such as age, gender, and tumor stage at diagnosis), their effect was evaluated through a multivariable (adjusted) Cox proportional hazards analysis (Cox D R., Journal of the Royal Statistical Society Series B (Methodological). 1972; 34(2):187-220) using the Tang et al. dataset, which demonstrated that these covariates are not predictive of treatment response and do not affect predictive ability of the model (FIG. 5C). To confirm this result, a stratified Kaplan-Meier survival analysis was performed, in which the Tang et al. validation cohort was stratified into patient groups based on (i) age (<median age and >=median age), (ii) gender (female and male), and (iii) tumor stage at diagnosis (stage I and stages II and III), which confirmed the ability of the systems and methods disclosed herein to predict a chemotherapy response does not depend on commonly utilized covariates and is indicative of a therapeutic response to carboplatin-paclitaxel (FIGS. 12A-12C).

Finally, to ensure that the systems and methods disclosed herein are not affected by markers of overall tumor aggressiveness, whether known prognostic signatures of lung cancer aggressiveness can predict a carboplatin-paclitaxel response or affect the predictive ability of the systems and methods disclosed herein was examined. For this analysis, known prognostic signatures of lung cancer aggressiveness were selected, including (i) Larsen et al. (Larsen et al., Clin. Can. Res. 2007; 13(10):2946-54) (54 prognostic markers), (ii) Beer et al. (Beer et al., Nature medicine. 2002; 8(8):816-24) (50 prognostic markers), and (iii) Tang et al. (Tang et al., Clin. Can. Res. 2013; 19(6):1577-86) (12 prognostic markers) (FIG. 5D), which were utilized in a multivariable Cox proportional hazards analysis, as described above. The analysis demonstrated that these prognostic signatures were not predictive of a carboplatin-paclitaxel response and do not affect the predictive ability of the 7 candidate pathways (FIG. 5D).

Model generalizability: In order to test the general applicability of the systems and methods disclosed herein, they were examined across additional chemotherapy combinations and cancer types. In particular, pathCHEMO was used to determine (i) the cisplatin-vinorelbine response in lung adenocarcinoma; (ii) the cisplatin-vinorelbine response in lung squamous cell carcinoma; and (iii) the folinic acid, fluorouracil, and oxaliplatin (FOLFOX) response in colorectal adenocarcinoma (Tables 3-5).

First, the systems and methods disclosed herein were applied to additional chemo combinations (such as cisplatin-vinorelbine), which were administered to lung adenocarcinoma (LUAD) patients, identifying a set of three (3) molecular pathways as markers of cisplatin-vinorelbine resistance (NES=2.51, p-value<0.001) (FIG. 14A). These pathways include (i) metabolism of nucleotides (Fabregat et al., Nucleic Acids Res. 2016; 44(D1):D481-7), (ii) actin Y (Nishimura D., Biotech Software & Internet Report: The Computer Software Journal for Scient. 2001; 2(3):117-20), and (iii) ribosome (Ogata et al., Nucleic Acids Res. 1999; 27(1):29-34) pathways, which differ from pathway markers of the carboplatin-paclitaxel response. Next, the predictions were validated using the Zhu et al. (Zhu et al., J. Clin. Oncol. 2010; 28(29):4417-24) patient cohort from the National Cancer Institute of Canada Clinical Trials Group (Table 3), which contains LUAD tumor samples (n=39) collected through surgery for patients that received adjuvant cisplatin-vinorelbine treatment, and the data demonstrate that the three candidate pathways predict poor and favorable cisplatin-vinorelbine responses in patients with LUAD (lung cancer-related death used as a clinical endpoint) using a Kaplan-Meier survival analysis (FIG. 6A, log-rank p-value=0.0048, hazard ratio=3.64).

Next, the systems and methods disclosed herein were applied to cisplatin-vinorelbine-treated lung squamous cell carcinoma (LUSC) patients, identifying a set of six (6) molecular pathways (NES=1.67, p-value<0.001) (FIG. 14B), including (i) neuroactive ligand-receptor interaction (Ogata et al., Nucleic Acids Res. 1999; 27(1):29-34), (ii) SLC-mediated transmembrane transport (Fabregat et al., Nucleic Acids Res. 2016; 44(D1):D481-7.), (iii) transport of mature mRNA derived from an intron-containing transcript (Fabregat et al., Nucleic Acids Res. 2016; 44(D1):D481-7), (iv) cytokine-cytokine receptor interaction (Ogata et al., Nucleic Acids Res. 1999; 27(1):29-34), (v) DNA repair (Fabregat et al., Nucleic Acids Res. 2016; 44(D1):D481-7), and (vi) translation (Fabregat et al., Nucleic Acids Res. 2016; 44(D1):D481-7) pathways. The predictions were validated using the Zhu et al. patient cohort (Zhu et al., J. Clin. Oncol. 2010; 28(29):4417-24) (Table 4), which contains LUSC tumor samples (n=26) collected through surgery, for patients that received adjuvant cisplatin-vinorelbine treatment, demonstrating that six candidate pathways can predict poor and favorable cisplatin-vinorelbine responses in patients with LUSC (lung cancer-related death used as clinical endpoint) using a Kaplan-Meier survival analysis (FIG. 6B, log-rank p-value=0.026, hazard ratio=7.94).

Finally, the systems and methods disclosed herein were applied to patients with colorectal adenocarcinoma (COAD) that received the FOLFOX (folinic acid, fluorouracil, and oxaliplatin) combination, identifying five (5) molecular pathways as markers of FOLFOX resistance (NES=2.02, p-value<0.001) (FIG. 14C). The pathways included (i) processing capped intron-containing pre mRNA (Fabregat et al., Nucleic Acids Res. 2016; 44(D1):D481-7), (ii) S phase (Fabregat et al., Nucleic Acids Res. 2016; 44(D1):D481-7), (iii) elongation and processing capped transcripts (Fabregat et al., Nucleic Acids Res. 2016; 44(D1):D481-7), (iv) protein metabolism (Fabregat et al., Nucleic Acids Res. 2016; 44(D1):D481-7), and (v) calcium signaling (Ogata et al., Nucleic Acids Res. 1999; 27(1):29-34) pathways. The predictions were evaluated using an independent patient cohort, Marisa et al. (Marisa et al., PLoS medicine. 2013; 10(5):e1001453) (Table 5) from the French National Cartes d′Identité des Tumeurs (CIT), which contains COAD tumor samples (n=23) collected through surgery followed by adjuvant treatment with FOLFOX and monitoring for further disease progression (locoregional or distant recurrence), which demonstrated that five candidate pathways can predict poor and favorable FOLFOX responses in patients with COAD using Kaplan-Meier survival analysis (FIG. 6C, log-rank p-value=0.01, hazard ratio=6.21).

These analyses demonstrate general applicability of the systems and methods disclosed herein across various chemotherapy combinations and cancer types, improving the field of personalized therapeutic advice for cancer patients and clinical decision support.

In view of the many possible embodiments to which the principles of the disclosure may be applied, it should be recognized that the illustrated embodiments are only examples of the disclosure and should not be taken as limiting in scope. Rather, the scope of the invention is defined by the following claims. We, therefore, claim as our invention all that comes within the scope and spirit of these claims.

1. A method of treating a subject with lung cancer, comprising:
(i) measuring expression and/or methylation of lung cancer-related molecules from lung cancer-related pathways in a sample obtained from a subject, wherein the lung cancer-related pathways comprise:
(a) chemokine receptor, mitotic cell cycle, immune network for immunoglobulin A (IgA) production, RNA degradation, mRNA splicing, protein metabolism, and G alpha signaling pathways;
(b) nucleotide metabolism, actin Y, and ribosome pathways; or
(c) cytokine-cytokine receptor interaction, neuroactive ligand-receptor interaction, DNA repair, SLC-mediated transmembrane transport, translation, and transport of mature mRNA derived from an intron-containing transcript pathways; and
(ii) administering:
(a) at least one of surgery, radiation therapy, targeted therapy, immunotherapy, or palliative care to the subject with lung cancer, thereby treating the subject, wherein:
(1) expression of the lung cancer-related molecules differs from a control representing expression for the lung cancer-related molecules expected in a sample from a subject who positively responds to a chemotherapy; and/or
(2) methylation of the lung cancer-related molecules differs from a control representing methylation for the lung cancer-related molecules expected in a sample from a subject who positively responds to a chemotherapy; or
(b) a chemotherapy, thereby treating the subject, wherein
(1) expression of the lung cancer-related molecules is similar to a control representing expression for the lung cancer-related molecules expected in a sample from a subject who positively responds to the chemotherapy; and/or
(2) methylation of the lung cancer-related molecules is similar to a control representing methylation for the lung cancer-related molecules expected in a sample from a subject who positively responds to the chemotherapy.

2. A method of identifying a subject with lung cancer who will respond positively to a chemotherapy, comprising:
measuring expression and/or methylation of lung cancer-related molecules from lung cancer-related pathways in a sample obtained from a subject, wherein the lung cancer-related pathways comprise:
(a) chemokine receptor, mitotic cell cycle, immune network for immunoglobulin A (IgA) production, RNA degradation, mRNA splicing, protein metabolism, and G alpha signaling pathway;
(b) nucleotide metabolism, actin Y, and ribosome pathways; or
(c) cytokine-cytokine receptor interaction, neuroactive ligand-receptor interaction, DNA repair, SLC-mediated transmembrane transport, translation, and transport of mature mRNA derived from an intron-containing transcript pathway,
wherein:
expression of the lung cancer-related molecules is similar to a control representing expression for the lung cancer-related molecules expected in a sample from a subject who positively responds to a chemotherapy; and/or
methylation of the lung cancer-related molecules is similar to a control representing methylation for the lung cancer-related molecules expected in a sample from a subject who positively responds to a chemotherapy,
thereby identifying a subject with lung cancer who will respond positively to the chemotherapy.

3. The method of claim 1 or claim 2, wherein the lung cancer-related molecules from the lung cancer-related pathways comprise:
(i) C-C motif chemokine ligand 22 (CCL22) from the chemokine receptor pathway, fibroblast growth factor receptor 1 oncogene partner (FGFR1OP) from the mitotic cell cycle pathway, C-C motif chemokine receptor 9 (CCR9) from the immune network for IgA production and chemokine receptor pathway, LSM7 from the RNA degradation pathway, RNA polymerase II subunit C (POLR2C) from the RNA splicing pathway, chaperonin containing TCP1 subunit 4 (CCT4) from the protein metabolism pathway, and phosphodiesterase 7A (PDE7A) from the G alpha signaling pathway;
(ii) deoxythymidylate kinase (DTYMK) from the nucleotide metabolism pathway, actin-related protein 2/3 complex subunit 1A (ARPC1A) from the actin Y pathway, and ribosomal protein lateral stalk subunit P2 (RPLP2) from the ribosome pathway; or
(iii) C-C motif chemokine 11 (CCL11) from the cytokine-cytokine receptor interaction pathway, gamma-aminobutyric acid receptor alpha-1 (GABRA1) from the neuroactive ligand-receptor interaction pathway, excision repair cross-complementation group 1 (ERCC1) from the DNA repair pathway, solute carrier family 44 member 4 (SLC44A4) from the solute carrier (SLC)-mediated transmembrane transport pathway, ribosomal protein L14 (RPL14) from the translation pathway, and U2 small nuclear RNA auxiliary factor 1 (U2AF1) from the transport of mature mRNA derived from an intron-containing transcript pathway.

4. The method of claim 1, wherein the chemotherapy comprises carboplatin, paclitaxel, cisplatin, vinorelbine, or a combination thereof.

5. The method of claim 1, wherein:
(i) the chemotherapy comprises carboplatin and paclitaxel, and:
(a) the lung cancer-related pathways comprise chemokine receptor, mitotic cell cycle, immune network for IgA production, RNA degradation, mRNA splicing, protein metabolism, and G alpha signaling pathway; or
(b) the lung cancer-related molecules comprise CCL22, CCR9, POLR2C, LSM7, FGFR1OP, PDE7A, and CCT4; or
(ii) the chemotherapy comprises cisplatin and vinorelbine, and:
(a) the lung cancer-related pathways comprise nucleotide metabolism, actin Y, and ribosome pathways;
(b) the lung cancer-related pathways comprise cytokine-cytokine receptor interaction, neuroactive ligand-receptor interaction, DNA repair, SLC-mediated transmembrane transport, translation, and transport of mature mRNA derived from an intron-containing transcript pathways;
(c) the lung cancer-related molecules comprise DTYMK, ARPC1A, and RPLP2; and/or
(d) the lung cancer-related molecules comprise CCL11, GABRA1, ERCC1, SLC44A4, RPL14, and U2AF1.

6. The method of claim 1, wherein:
(i) lung cancer-related molecules with similar expression to a control comprise:
(a) lung cancer-related molecules from lung cancer-related pathways comprising chemokine receptor, mitotic cell cycle, immune network for IgA production, mRNA splicing, protein metabolism, and G alpha signaling pathways; and/or
(b) CCL22, CCR9, POLR2C, FGFR1OP, CCT4, and PDE7A; and
(ii) lung cancer-related molecules with similar methylation to a control comprise:
(a) lung cancer-related molecules from lung cancer-related pathways comprising chemokine receptor, mitotic cell cycle, immune network for IgA production, mRNA splicing, protein metabolism, and RNA degradation pathway; and/or
(b) CCL22, CCR9, POLR2C, FGFR1OP, CCT4, and LSM7.

7. (canceled)

8. The method of claim 5, wherein the lung cancer comprises lung adenocarcinoma, and:
(i) the lung cancer-related pathways comprise nucleotide metabolism, actin Y, and ribosome pathways; and/or
(ii) the lung cancer-related molecules comprise DTYMK, ARPC1A, and RPLP2.

9. The method of claim 8, wherein:
(i) the lung cancer-related molecules with similar expression to a control comprise:
(a) lung cancer-related molecules from lung cancer-related pathways comprising nucleotide metabolism, actin Y, and ribosome pathways; and/or
(b) DTYMK, ARPC1A, and RPLP2; and
(ii) the lung cancer-related molecules with similar methylation to a control comprise:
(a) lung cancer-related molecules from the ribosome pathway; and/or
(b) RPLP2.

10. (canceled)

11. The method of claim 5, wherein the lung cancer comprises lung squamous cell carcinoma, and:
(i) the lung cancer-related pathways comprise cytokine-cytokine receptor interaction, neuroactive ligand-receptor interaction, DNA repair, SLC-mediated transmembrane transport, translation, and transport of mature mRNA derived from an intron-containing transcript pathways; and/or
(ii) the lung cancer-related molecules comprise CCL11, GABRA1, ERCC1, SLC44A4, RPL14, and U2AF1.

12. The method of claim 11, wherein:
(i) the lung cancer-related molecules with similar expression to a control comprise:
(a) lung cancer-related molecules from lung cancer-related pathways comprising cytokine-cytokine receptor interaction, DNA repair, and transport of mature mRNA derived from an intron-containing transcript pathways; and/or
(b) CCL11, ERCC1, and U2AF1; and
(ii) the lung cancer-related molecules with similar methylation to a control comprise:
(a) lung cancer-related molecules from lung cancer-related pathways comprising cytokine-cytokine receptor interaction, neuroactive ligand-receptor interaction, SLC-mediated transmembrane transport, and translation pathways; and/or
(b) CCL11, GABRA1, SLC44A4, and RPL14.

13. A method of treating a subject with colorectal cancer, comprising:
(i) measuring expression and/or methylation of colorectal cancer-related molecules from colorectal cancer-related pathways in a sample obtained from a subject, wherein the colorectal cancer-related pathways comprise elongation and processing of capped transcripts; processing of capped intron containing pre-mRNA; protein metabolism; S phase; and calcium signaling pathways; and
(ii) administering:
(a) at least one of surgery, radiation therapy, targeted drug therapy, immunotherapy, or palliative care to the subject with colorectal cancer, thereby treating the subject, wherein:
(1) expression of the colorectal cancer-related molecules differs from a control representing expression for the colorectal cancer-related molecules expected in a sample from a subject who does not have colorectal cancer; and/or
(2) methylation of the colorectal cancer-related molecules differs from to a control representing methylation for the colorectal cancer-related molecules expected in a sample from a subject who does not have colorectal cancer; or
(b) chemotherapy, thereby treating the subject, wherein:
(1) expression of the colorectal cancer-related molecules is similar to a control representing expression for the colorectal cancer-related molecules expected in a sample from a subject who does not have colorectal cancer; and/or
(2) methylation of the colorectal cancer-related molecules is similar to a control representing methylation for the colorectal cancer-related molecules expected in a sample from a subject who does not have colorectal cancer.

14. (canceled)

15. The method of claim 13, wherein the colorectal cancer-related molecules from the colorectal cancer-related pathways comprise splicing factor 3b subunit 3 (SF3B3) from the elongation and processing of capped transcripts pathway, pre-mRNA processing factor 6 (PRPF6) from the processing of capped intron containing pre mRNA pathway, prefoldin subunit 1 (PFDN1) from the protein metabolism pathway, cell division cycle 25B (CDC25B) from the S phase pathway, and myosin light chain kinase 3 (MYLK3) from the calcium signaling pathway.

16. (canceled)

17. The method of claim 13, wherein the chemotherapy comprises folinic acid, fluorouracil, oxaliplatin, or a combination thereof.

18. (canceled)

19. The method of any one of claim 13, wherein:
(i) the colorectal cancer-related molecules with similar expression to a control comprise:
(a) colorectal cancer-related molecules from colorectal cancer-related pathways comprising elongation and processing of capped transcripts, processing of capped intron containing pre-mRNA, S phase, and calcium signaling pathways; and/or
(b) SF3B3, PRPF6, CDC25B, and MYLK3; and
(ii) the colorectal cancer-related molecules with similar methylation to a control comprise:
(a) colorectal cancer-related molecules from colorectal cancer-related pathways comprising processing of capped intron containing pre-mRNA, protein metabolism, S phase, and calcium signaling pathways; and/or
(b) PFDN1, CDC25B, and MYLK3.

20. (canceled)

21. The method of claim 1, wherein the subject who responds positively to the chemotherapy comprises a subject who does not develop resistance to the chemotherapy.

22. The method of claim 1, wherein the treating the subject only occurs where the subject is identified as a subject who responds positively or does not respond positively to chemotherapy with a p value of at least 0.01.

23. The method of any one of claim 2, wherein the subject is identified as a subject who will respond positively to chemotherapy with a p value of at least 0.01.

24. (canceled)

25. The method of any one of claim 1, wherein the sample is a lung cancer.

26. (canceled)

27. The method of claim 1, wherein the expression comprises mRNA expression and methylation comprises DNA methylation.

28. The method of claim 2, wherein a subject that responds positively to a chemotherapy is a subject
with a cancer that is reduced in size by at least 20%, at least 50%, at least 80%, at least 90%, at least 95%, at least 98%, or even at least 100%, following administration of the chemotherapy, as compared to no treatment with the chemotherapy;
with a metastasis that is reduced in size by at least 20%, at least 50%, at least 80%, at least 90%, at least 95%, at least 98%, or even at least 100%, following administration of the chemotherapy, as compared to no treatment with the chemotherapy;
has an increase in survival time following administration of the chemotherapy, as compared to no treatment with the chemotherapy;
has a reduction of at least 65%, at least 85%, at least 90%, at least 95%, or at least 98%, in developing resistance to the chemotherapy, or
combinations thereof, for example within one year of starting treatment with the chemotherapy.
